(19) United States
(12) Patent Application Publication
(10) Pub. No.: US 2008/0194482 A1
(43) Pub. Date: Aug. 14, 2008

(54) Selective Apac2 Receptor Peptide Agonists

(76) Inventors: Jorge Alsina-Fernandez, Indianapolis, IN (US); Bengt Krister Bokvist, Hamburg (DE); Lianshan Zhang, Carmel, IN (US)

Correspondence Address: ELI LILLY & COMPANY, PATENT DIVISION, P.O. BOX 6288, INDIANAPOLIS, IN, 46206-6288, US

(21) Appl. No.: 11/997,499

(22) PCT Filed: Jul. 28, 2006
(86) PCT No.: PCT/US2006/029440

§ 371 Date: Jan. 31, 2008

Related U.S. Application Data
(60) Provisional application No. 60/707,493, filed on Aug. 11, 2005.
(60) Provisional application No. 60/730,353, filed on Oct. 26, 2005.
(60) Provisional application No. 60/740,594, filed on Nov. 29, 2005.

(51) Int. Cl. A61K 38/17; C07K 14/435; C07K 7/06; A61P 3/10; A61K 38/08

(52) U.S. Cl. 514/12; 530/324; 530/327; 530/345; 514/15

(57) ABSTRACT

The present invention encompasses peptides that selectively activate the VPAC2 receptor and are useful in the treatment of diabetes.

The present invention relates to selective VPAC2 receptor peptide agonists.

Type 2 diabetes, or non-insulin dependent diabetes mellitus (NIDDM), is the most common form of diabetes, affecting 90% of people with diabetes. With NIDDM, patients have impaired β-cell function resulting in insufficient insulin production and/or decreased insulin sensitivity. If NIDDM is not controlled, excess glucose accumulates in the blood, resulting in hyperglycemia. Over time, more serious complications may arise including renal dysfunction, cardiovascular problems, visual loss, lower limb ulceration, neuropathy, and ischemia. Treatments for NIDDM include improving diet, exercise, and weight control as well as using a variety of oral medications. Individuals with NIDDM can initially control their blood glucose levels by taking such oral medications. These medications, however, do not slow the progressive loss of β-cell function that occurs in NIDDM patients and, thus, are not sufficient to control blood glucose levels in the later stages of the disease. Also, treatment with currently available medications exposes NIDDM patients to potential side effects such as hypoglycemia, gastrointestinal problems, fluid retention, oedema, and/or weight gain.

Pituitary adenylate cyclase-activating peptide (PACAP) and vasoactive intestinal peptide (VIP) belong to the same family of peptides as secretin and glucagon. PACAP and VIP work through three G-protein-coupled receptors that exert their action through the cAMP-mediated and other Ca²⁺-mediated signal transduction pathways. These receptors are known as the PACAP-preferring type 1 (PAC1) receptor (Isobe, et al., Regul. Pept., 110:213-217 (2003); Ogi, et al., Biochem. Biophys. Res. Commun., 196:1511-1521 (1993)) and the two VIP-shared type 2 receptors (VPAC1 and VPAC2) (Sherwood et al., Endocr. Rev., 21:619-670 (2000); Hammar et al., Pharmacol Rev, 50:265-270 (1998); Couvineau, et al., J. Biol. Chem., 278:24759-24766 (2003); Sreedharan, et al., Biochem. Biophys. Res. Comunun., 193:546-553 (1993); Lutz, et al., FEBS Lett., 458: 197-203 (1999); Adamou, et al., Biochem. Biophys. Res. Comunun., 209: 385-392 (1995)). A series of PACAP analogues is disclosed in U.S. Pat. No. 6,242,563 and WO 2000/05260.

PACAP has comparable activities towards all three receptors, whilst VIP selectively activates the two VPAC receptors (Tsutsumi et al., Diabetes, 51:1453-1460 (2002)). Both VIP (Eriksson et al., Peptides, 10: 481-484 (1989)) and PACAP (Filipsson et al., JCEM, 82:3093-3098 (1997)) have been shown to not only stimulate insulin secretion in man when given intravenously but also increase glucagon secretion and hepatic glucose output. As a consequence, PACAP or VIP stimulation generally does not result in a net improvement of glycemia. Activation of multiple receptors by PACAP or VIP also has broad physiological effects on nervous, endocrine, cardiovascular, reproductive, muscular, and immune systems (Gozes et al., Curr. Med. Chem., 6:1019-1034 (1999)). It appears that VIP-induced watery diarrhoea in rats is mediated by only one of the VPAC receptors, VPAC1 (Ito et al., Peptides, 22:1139-1151 (2001); Tsutsumi et al., Diabetes, 51:1453-1460 (2002)). The VPAC1 and PAC1 receptors are expressed on α-cells and hepatocytes and, thus, are most likely involved in the effects on hepatic glucose output.

Exendin-4 is found in the salivary excretions from the Gila Monster, Heloderma Suspectum, (Eng et al., J. Biol. Chem., 267(11):7402-7405 (1992)). It is a 39 amino acid peptide, which has glucose dependent insulin secretagogue activity.

Recent studies have shown that peptides selective for the VPAC2 receptor are able to stimulate insulin secretion from the pancreas without gastrointestinal (GI) side effects and without enhancing glucagon release and hepatic glucose output (Tsutsumi et al., Diabetes, 51:1453-1460 (2002)). Peptides selective for the VPAC2 receptor were initially identified by modifying vasoactive intestinal peptide (VIP) and/or pituitary adenylate cyclase-activating polypeptide (PACAP). (See, for example, Xia et al., J Pharmacol Exp Ther., 281:629-633 (1997); Tsutsumi et al., Diabetes, 51:1453-1460 (2002); WO 01/23420; and WO 2004/06839.)

Many of the VPAC2 receptor peptide agonists reported to date have, however, less than desirable potency, selectivity, and/or stability profiles, which could impede their clinical viability. In addition, many of these peptides are not suitable for commercial candidates as a result of stability issues associated with the polypeptides in formulation, as well as issues with the short half-life of these polypeptides in vivo. There is, therefore, a need for new therapies, which overcome the problems associated with current medications for NIDDM.

The present invention seeks to provide improved compounds that are selective for the VPAC2 receptor and which induce insulin secretion from the pancreas only in the presence of high blood glucose levels. The compounds of the present invention are peptides, which are believed to also improve beta cell function. These peptides can have the physiological effect of inducing insulin secretion without GI side effects or a corresponding increase in hepatic glucose output and also generally have enhanced selectivity, potency, and/or in vivo stability of the peptide compared to known VPAC2 receptor peptide agonists.

According to a first aspect of the invention, there is provided a VPAC2 receptor peptide agonist comprising a sequence selected from:

SEQ ID NO: 17

HSDAVFTEQY(OMe)TRAibRAibQLAAAibOrnY(OMe)LQSIK
AibOrn;

SEQ ID NO: 18

HSDAVFTEK(CO(CH₂)₂SH)Y(OMe)TOrnLRAibQVAAAibOrn
YLQSIOrnOrn;

SEQ ID NO: 19

HSDAVFTEQY(OMe)TOrnLRAibQVAAAibOrnYLQSIOrnK(W)
Orn;

SEQ ID NO: 20

HSDAVFTEQY(OMe)TOrnLRAibQVAAAibK(CO(CH₂)₂SH)YLQ
SIOrnOrn;

SEQ ID NO: 21

HSDAVFTEQY(OMe)TOrnLRAibQVAAK(CO(CH₂)₂SH)OrnYLQ
SIOrnOrn;

SEQ ID NO: 22

HSDAVFTEQY(OMe)TOrnLRAibQVCAAibOrnYLQSIOrnOrn;

SEQ ID NO: 23

HSDAVFTEQY(OMe)TOrnLRCQVAAAibOrnYLQSIOrnOrn;

SEQ ID NO: 24

HSDAVFTEQY(OMe)TOrnLRAibQLAAAibOrnYLQSIOrnOrn;

SEQ ID NO: 25

HSDAVFTEQY(OMe)TOrnLRAibQVAAAibOrnYAibQSIOrnOrn;

SEQ ID NO: 26

HSDAVFTEQY(OMe)TOrnLRAibQVAAAibOrnYLQAibIOrnOrn;

SEQ ID NO: 27

HSDAVFTEQY(OMe)TOrnLRAibQVAAbuAibOrnYLQAibIOrnOrn;

SEQ ID NO: 28

HSDAVFTEQY(OMe)TOrnLRAibQLAAAibOrnYLQAibIOrnOrn;

SEQ ID NO: 29

HSDAVFTEQY(OMe)TOrnLRAibQLAAAibOrnYAibQAibIOrnOrn;

SEQ ID NO: 30

HSDAVFTEQY(OMe)TOrnLRAibQLAAbuAibOrnYAibQSIOrnOrn;

SEQ ID NO: 31

HSDAVFTEQY(OMe)TOrnLRAibQLAAbuAibOrnYLQSIOrnOrn;

SEQ ID NO: 32

HSDAVFTEQY(OMe)TOrnLRAibQLAAbuAibOrnYAibQAibIOrn
Orn;

SEQ ID NO: 33

HSDAVFTEQY(OMe)TOrnLRAibQLAAAibOrnYAibQSIOrnOrn;

SEQ ID NO: 34

HSDAVFTEQY(OMe)TOrnLRK(W)QVAAAibOrnYLQSIOrnOrn;

SEQ ID NO: 35

HSDAVFTEQY(OMe)TOrnLRAibQVAAAibOrnYLK(W)SIOrnOrn;

SEQ ID NO: 36

HSDAVFTEQY(OMe)TOrnLRAibQK(W)AAAibOrnYLQSIOrnOrn;

SEQ ID NO: 37

HSDAVFTEQY(OMe)TOrnLRK(CO(CH₂)₂SH)QVAAAibOrnYLQ
SIOrnOrn:

SEQ ID NO: 38

HSDAVFTEQY(OMe)TOrnLRAibQVAAAibK(W)YLQSIOrnOrn;

SEQ ID NO: 39

HSDAVFTEQY(OMe)TOrnLRAibQVAAAibCYLQSIOrnOrn;

SEQ ID NO: 40

HSDAVFTEQY(OMe)TOrnLRAibQLAAbuAibOrnYLQAibOrnOrn;

SEQ ID NO: 41

HSDAVFTEQY(OMe)TOrnLRAibQVAAAibOrnYLQSK(W)OrnOrn;

SEQ ID NO: 42

HSDAVFTEQY(OMe)TOrnLRAibQLAAbuAibOrnYLQAibIOrnC
Orn;

SEQ ID NO: 43

HSDAVFTEQY(OMe)TOrnLRAibQLAAbuAibOrnYLQAibCOrn
Orn;

SEQ ID NO: 44

HSDAVFTEQY(OMe)TOrnLRAibQCAAbuAibOrnYLQAibIOrnOrn;

SEQ ID NO: 45

HSDAVFTEQY(OMe)TOrnLRCQLAAbuAibOrnYLQAibIOrnOrn;

SEQ ID NO: 92

HSDAVFTEQY(OMe)TOrnLRAibQVK(CO(CH₂)₂SH)AAibOrn
YLQSIOrnOrn;

SEQ ID NO: 93

HSDAVFTEQY(OMe)TOrnLRAibQLAAbuAibOrnYLQSIOrnCOrn;

SEQ ID NO: 94

HSDAVFTEQY(OMe)TOrnLRAibQLAAbuAibOrnYLQSCOrnOrn;

SEQ ID NO: 95

HSDAVFTEQY(OMe)TOrnLRAibQLAAbuAibOrnYLQAibIOrn
K(CO(CH₂)₂SH)Orn;

SEQ ID NO: 96

HSDAVFTEQY(OMe)TOrnLRAibQLAAbuAibOrnYLQSIOrn
K(CO(CH₂)₂SH)Orn;

SEQ ID NO: 97

HSDAVFTEQY(OMe)TOrnLRK(W)QLAAbuAibOrnYLQAibIOrn
Orn;

SEQ ID NO: 98

HSDAVFTEQY(OMe)TOrnLRAibQLAAAibOrnYLQSIOrnOrnC;

SEQ ID NO: 99

HSDAVFTEQY(OMe)TOrnLRAibQVAAAibOrnYLQSIOrnOrnC;

SEQ ID NO: 100

HSDAVFTEQY(OMe)TOrnLRAibQLAAbuAibOrnYLQSIOrnOrnC;

SEQ ID NO: 101

HSDAVFTEQY(OMe)TOrnLRAibQLAAbuAibOrnY(OMe)LQAibI
OrnOrn;

SEQ ID NO: 102

HSDAVFTEQY(OMe)TOrnLRAibQLAAbuAibOrnY(OMe)LQAibI
OrnCOrn;

SEQ ID NO: 103

HSDAVFTEQY(OMe)TOrnLRAibQCAAbuAibOrnY(OMe)LQAibI
OrnOrn;

SEQ ID NO: 104

HSDAVFTEQY(OMe)TOrnLRAibQLAAbuAibOrnYLQAibIOrn
OrnC;

SEQ ID NO: 105

HSDAVFTEQY(OMe)TOrnLRAibQLAAbuAibOrnY(OMe)LQSI
OrnOrn;

SEQ ID NO: 106

HSDAVFTEQY(OMe)TOrnLRAibQCAAbuAibOrnY(OMe)LQSI
OrnOrn;

SEQ ID NO: 107

HSDAVFTEQY(OMe)TOrnLRAibQLAAbuAibOrnY(OMe)LQSI
OrnCOrn;

SEQ ID NO: 108

HSDAVFTEQY(OMe)TOrnLRAibQLAbuAAibOrnYLQSIOrnOrn;

SEQ ID NO: 109

HSDAVFTEQY(OMe)TOrnLRAibQK(CO(CH₂)₂SH)AAbu
AibOrnYLQAibIOrnOrn;

SEQ ID NO: 110

HSDAVFTEQY(OMe)TOrnLRAibQK(W)AAbuAibOrnYLQ
AibIOrnOrn;

SEQ ID NO: 145

HSDAVFTDNYTRLRKQVAAKKYLQSIKNKRQ;

SEQ ID NO: 146

HSDAVFTDNYTLLRAibQVAAAibKYLQSIOrnNOrn;

SEQ ID NO: 147

HSDAVFTDNYTQLRAibQVAAAibKYLQSIOrnNOrn;

SEQ ID NO: 148

HSDAVFTDNYTFLRAibQVAAAibKYLQSIOrnNOrn;

SEQ ID NO: 149

HSDAVFTDNYTOrnLRAibQVAAAibKYLQCIOrnNOrn;

SEQ ID NO: 150

HSDAVFTDNYTOrnLRAibQVAACOrnYLQSIOrnNOrn;

SEQ ID NO: 151

HSDAVFTDNYTOrnLRAibQVAAAibKYLQSSOrnNOrn;

SEQ ID NO: 152

HSDAVFTDNYTOrnLRAibQVAAAibKYLSSIOrnNOrn;

SEQ ID NO: 153

HSDAVFTDNYTOrnLRAibQVAAAibKYSQSIOrnNOrn;

SEQ ID NO: 154

HSDAVFTDNYTOrnLRAibQVAAAibKSLQSIOrnNOrn;

SEQ ID NO: 155

HSDAVFTDNYTOrnLRAibQVAAAibSYLQSIOrnNOrn;

SEQ ID NO: 156

HSDAVFTDNYTOrnLRAibQVSAAibKYLQSIOrnNOrn;

SEQ ID NO: 157

HSDAVFTDNYTOrnLRAibQSAAAibKYLQSIOrnNOrn;

SEQ ID NO: 158

HSDAVFTDNYTOrnLRAibSVAAAibKYLQSIOrnNOrn;

SEQ ID NO: 159

HSDAVFTDNYTOrnSRAibQVAAAibKYLQSIOrnNOrn;

SEQ ID NO: 160

HSDAVFTDSYTOrnLRAibQVAAAibKYLQSIOrnNOrn;

SEQ ID NO: 161

HSDAVFTDNYThRLRAibQVAAAibKYLQSIKNKRY;

SEQ ID NO: 162

HSDAVFTDNYTRLRAibQVAAAibKYLQSIKAibOrn;

SEQ ID NO: 163

HSDAVFTDNY(OMe)TRLRAibQVAAAibKYLQSIKNKRY;

SEQ ID NO: 164

HSEAVFTENYTOrnLRAibQVAAAibKYLQSIOrnNOrn;

SEQ ID NO: 165

HSDAVFTDQYTOrnLRAibQVAAAibKYLQSIOrnQOrn;

SEQ ID NO: 166

HSDAVFTDNYTRLLAKLALQKYLQSIOrnNOrn;

SEQ ID NO: 167

HSDAVFTDNYTOrnLLAKLALQKYLQSIOrnNOrn;

SEQ ID NO: 168

HSEAVFTEQYTOrnLRAibQVAAAibOrnYLQSIOrnOrn;

SEQ ID NO: 169

HSDAVFTDNYTOrnLRAibQVASAibKYLQSIOrnNOrn;

SEQ ID NO: 170

HSEAVFTEQY(OMe)TOrnLRAibQLAAAibOrnYLQSIOrnOrn;

SEQ ID NO: 171

HSDAVFTDQY(OMe)TOrnLRAibQLAAAibOrnYLQSIOrnOrn;

SEQ ID NO: 172

HSDAVFTDQYTOrnLRAibQLAAAibOrnYLQSIOrnOrn;

SEQ ID NO: 173

HSDAVFTDQYTOrnLRAibQVAAAibOrnYLQSIOrnOrn;

SEQ ID NO: 174

HSDAVFTDNYTOrnLRAibQVAAAibOrnYLQSIOrnOrn;

SEQ ID NO: 175

HSDAVFTEQY(OMe)TOrnLRAibQVAAAibOrnYLQSIOrnOrn;

SEQ ID NO: 176

HSDAVFTDNYTRAibRAibQVAAAibKYLQSIKAibK;

SEQ ID NO: 177

HSDAVFTDQYTRAibRAibQVAAAibKYLQSIKAibK;

SEQ ID NO: 178

HSDAVFTDQYTRAibRAibQLAAAibKYLQSIKAibK;

SEQ ID NO: 179

HSDAVFTDQY(OMe)TRAibRAibQLAAAibKYLQSIKAibK;

SEQ ID NO: 180

HSEAVFTEQY(OMe)TRAibRAibQLAAAibKYLQSIKAibK;

SEQ ID NO: 181

HSDAVFTEQY(OMe)TOrnLRAibQVAAAibOrnYLCSIOrnOrn;

SEQ ID NO: 182

HSDAVFTEQY(OMe)TOrnLRAibQVAAAibOrnYL
K(CO(CH₂)₂SH)SIOrnOrn;

SEQ ID NO: 183

HSDAVFTEQY(OMe)TOrnLRAibQVACAibOrnYLQSIOrnOrn;

SEQ ID NO: 184

HSDAVFTEQY(OMe)TOrnLRAibQVAK(CO(CH₂)₂SH)AibOrn
YLQSIOrnOrn;

SEQ ID NO: 185

HSDAVFTEQY(OMe)TOrnLRAibCVAAAibOrnYLQSIOrnOrn;

SEQ ID NO: 186

HSDAVFTDNYTOrnLRK(W)QVAAAibKYLQSIOrnNOrn;

SEQ ID NO: 187

HSDAVFTEQY(OMe)TOrnLRAibQLAAAibOrnY(OMe)LQ
SIOrnOrn;

SEQ ID NO: 188

HSDAVFTEQY(OMe)TOrnLRAibQVAAAibOrnYLQSCOrnOrn;

SEQ ID NO: 189

HSDAVFTEQY(OMe)TOrnLRAibQCAAAibOrnYLQSIOrnOrn;

SEQ ID NO: 190

HSDAVFTEQY(OMe)TOrnLRAibQVAAAibOrnYLQSIOrnCOrn;

SEQ ID NO: 191

HSDAVFTEQY(OMe)TOrnLRAibQVAAAibOrnYLQCIOrnOrn;

SEQ ID NO: 192

HSDAVFTECY(OMe)TOrnLRAibQVAAAibOrnYLQSIOrnOrn;

SEQ ID NO: 193

HSDAVFTEQY(OMe)TOrnCRAibQVAAAibOrnYLQSIOrnOrn;

SEQ ID NO: 194

HSDAVFTEQY(OMe)TOrnLRAibQVAAAibOrnYLQS
K(CO(CH₂)₂SH)OrnOrn;

SEQ ID NO: 195

HSDAVFTEQY(OMe)TOrnLRAibQVAAAibOrnYLQ
K(CO(CH₂)₂SH)IOrnOrn;

SEQ ID NO: 196

HSDAVFTEQY(OMe)TOrnLRAibQK(CO(CH₂)₂SH)AAAibOrnYL
QSIOrnOrn;

SEQ ID NO: 197

HSDAVFTEQY(OMe)TOrnLRAibK(CO(CH₂)₂SH)VAAAibOrnY
LQSIOrnOrn;

SEQ ID NO: 198

HSDAVFTEQY(OMe)TOrnLRAibQVAAAibOrnYLQSIOrn
K(CO(CH₂)₂SH)Orn; and

SEQ ID NO: 199

HSDAVFTEQY(OMe)TOrnK(CO(CH₂)₂SH)RAibQVAAAibOrn
YLQSIOrnOrn;

and a C-terminal extension wherein the N-terminus of the C-terminal extension is linked to the C-terminus of the peptide sequence and wherein the C-terminal extension comprises an amino acid sequence of the formula:

Formula 3

(SEQ ID NO: 3)

Xaa₁-Xaa₂-Xaa₃-Xaa₄-Xaa₅-Xaa₆-Xaa₇-Xaa₈-Xaa₉-
Xaa₁₀-Xaa₁₁-Xaa₁₂

wherein:
Xaa₁ is: Gly, Cys, or absent;
Xaa₂ is: Gly, Arg, or absent;
Xaa₃ is: Pro, Thr, or absent;
Xaa₄ is: Ser, or absent;
Xaa₅ is: Ser, or absent;
Xaa₆ is: Gly, or absent;
Xaa₇ is: Ala, or absent;
Xaa₈ is: Pro, or absent;
Xaa₉ is: Pro, or absent;
Xaa₁₀ is: Pro, or absent;
Xaa₁₁ is: Ser, Cys, or absent; and
Xaa₁₂ is: Cys, or absent;
   wherein at least five of Xaa₁ to Xaa₁₂ of the C-terminal extension are present and wherein if Xaa₁, Xaa₂, Xaa₃, Xaa₄, Xaa₅, Xaa₆, Xaa₇, Xaa₈, Xaa₉, Xaa₁₀, or Xaa₁₁ is absent, the next amino acid present downstream is the next amino acid in the C-terminal extension and wherein the C-terminal amino acid may be amidated.

Preferably, at least six of Xaa₁ to Xaa₁₂ of the C-terminal extension of Formula 3 are present. More preferably, at least seven, eight, nine, ten, eleven, or all of Xaa₁ to Xaa₁₂ of the C-terminal extension are present.

More preferably, the C-terminal extension of the VPAC2 receptor peptide agonist is selected from:

| | |
|---|---|
| SEQ ID NO: 5 | GGPSSGAPPPS |
| SEQ ID NO: 6 | GGPSSGAPPPS-NH₂ |
| SEQ ID NO: 7 | GGPSSGAPPPC |
| SEQ ID NO: 8 | GGPSSGAPPPC-NH₂ |
| SEQ ID NO: 9 | GRPSSGAPPPS |
| SEQ ID NO: 10 | GRPSSGAPPPS-NH₂ |
| SEQ ID NO: 11 | GGPSSGAPPPCC |
| SEQ ID NO: 12 | GGPSSGAPPPCC-NH₂ |

The VPAC2 receptor peptide agonist sequence may further comprise a histidine residue at the N-terminus of the peptide.

Preferably, the VPAC2 receptor peptide agonist according to the first aspect of the present invention further comprises a N-terminal modification at the N-terminus of the peptide agonist wherein the N-terminal modification is selected from:
(a) addition of D-histidine, isoleucine, methionine, or norleucine;
(b) addition of a peptide comprising the sequence Ser-Trp-Cys-Glu-Pro-Gly-Trp-Cys-Arg (SEQ ID NO: 91) wherein the Arg is linked to the N-terminus of the peptide agonist;
(c) addition of C₁-C₁₆ alkyl optionally substituted with one or more substituents independently selected from aryl, C₁-C₆ alkoxy, —NH₂, —OH, halogen and —CF₃;
(d) addition of —C(O)R¹ wherein R¹ is a C₁-C₁₆ alkyl optionally substituted with one or more substituents independently selected from aryl, C₁-C₆ alkoxy, —NH₂, —OH, halogen, —SH and —CF₃; a aryl or aryl C₁-C₄ alkyl optionally substituted with one or more substituents independently selected from C₁-C₆ alkyl, C₂-C₆ alkenyl, C₂-C₆ alkynyl, C₁-C₆ alkoxy, —NH₂, —OH, halogen and —CF₃; —NR²R³ wherein R² and R³ are independently hydrogen, C₁-C₆ alkyl, aryl or aryl C₁-C₄ alkyl; —OR⁴ wherein R⁴is C₁-C₁₆ alkyl optionally substituted with one or more substituents independently selected from aryl, C₁-C₆ alkoxy, —NH₂, —OH, halogen and —CF₃, aryl or aryl C₁-C₄ alkyl optionally substituted with one or more substituents independently selected from C₁-C₆ alkyl, C₂-C₆ alkenyl, C₂-C₆ alkynyl, C₁-C₆ alkoxy, —NH₂, —OH, halogen and —CF₃; or 5-pyrrolidin-2-one;
(e) addition of —SO₂R⁵ wherein R⁵ is aryl, aryl C₁-C₄ alkyl or C₁-C₁₆ alkyl;
(f) formation of a succinimide group optionally substituted with C₁-C₆ alkyl or —SR⁶, wherein R⁶ is hydrogen or C₁-C₆ alkyl;
(g) addition of methionine sulfoxide;
(h) addition of biotinyl-6-aminohexanoic acid (6-aminocaproic acid); and
(i) addition of —C(═NH)—NH₂.

Preferably, the N-terminal modification is the addition of a group selected from: acetyl, propionyl, butyryl, pentanoyl, hexanoyl, methionine, methionine sulfoxide, 3-phenylpropionyl, phenylacetyl, benzoyl, norleucine, D-histidine, isoleucine, 3-mercaptopropionyl, biotinyl-6-aminohexanoic acid (6-aminocaproic acid), and —C(═NH)—NH₂. It is especially preferred that the N-terminal modification is the addition of acetyl or hexanoyl.

It will be appreciated by the person skilled in the art that VPAC2 receptor peptide agonists comprising various combinations of peptide sequence selected from SEQ ID NO: 17 to 45, 92 to 110 and 145 to 199, C-terminal extensions and N-terminal modifications as described herein, may be made based on the above disclosure.

It is preferred that the VPAC2 receptor peptide agonist according to the first aspect of the present invention comprises an amino acid sequence selected from:

| | | |
|---|---|---|
| Agonist | SEQ ID | |
| # | NO | Sequence |

| | | |
|---|---|---|
| P400 | 46 | C6- |
| | | HSDAVFTEQY(OMe)TOrnLRAibQVAAAibK(W)YLQSIOrn |
| | | OrnGGPSSGAPPPS-NH₂ |
| P416 | 47 | C6- |
| | | HSDAVFTEQY(OMe)TRAibRAibQLAAAibOrnY(OMe)LQSIK |
| | | AibOrnGGPSSGAPPPC-NH₂ |
| P450 | 48 | C6- |
| | | HSDAVFTEK(CO(CH₂)₂SH)Y(OMe)TOrnLRAibQVAAAibOrn |
| | | YLQSIOrnOrnGGPSSGAPPPS-NH₂ |
| P453 | 49 | C6-HSDAVFTEQY(OMe)TOrnLRAibQVAAAibOrnYLQSIOrn |
| | | K(W)OrnGGPSSGAPPPS-NH₂ |
| P459 | 50 | C6- |
| | | HSDAVFTEQY(OMe)TOrnLRAibQVAAAibCYLQSIOrnOrnG |
| | | GPSSGAPPPS-NH₂ |
| P471 | 51 | C6- |
| | | HSDAVFTEQY(OMe)TOrnLRAibQVAAAibK(CO(CH₂)₂SH)Y |
| | | LQSIOrnOrnGGPSSGAPPPS-NH₂ |
| P474 | 52 | C6- |
| | | HSDAVFTEQY(OMe)TOrnLRAibQVAAK(CO(CH₂)₂SH)OrnY |
| | | LQSIOrnOrnGGPSSGAPPPS-NH₂ |
| P477 | 53 | C6- |
| | | HSDAVFTEQY(OMe)TOrnLRAibQVCAAibOrnYLQSIOrnOrn |
| | | GGPSSGAPPPS-NH₂ |
| P482 | 54 | C6- |
| | | HSDAVFTEQY(OMe)TOrnLRCQVAAAibOrnYLQSIOrnOrnG |
| | | GPSSGAPPPS-NH₂ |
| P487 | 55 | C6- |
| | | HSDAVFTEQY(OMe)TOrnLRAibQLAAAibOrnYLQSIOrnOrn |
| | | GGPSSGAPPPS-NH₂ |
| P506 | 56 | C6- |
| | | HSDAVFTEQY(OMe)TOrnLRAibQVAAAibOrnYAibQSIOrn |
| | | OrnGGPSSGAPPPC-NH₂ |
| P508 | 57 | C6- |
| | | HSDAVFTEQY(OMe)TOrnLRAibQVAAAibOrnYLQAibIOrn |
| | | OrnGGPSSGAPPPC-NH₂ |
| P510 | 58 | C6- |
| | | HSDAVFTEQY(OMe)TOrnLRAibQVAAbuAibOrnYLQAibIOrn |
| | | OrnGGPSSGAPPPC-NH₂ |
| P514 | 59 | C6- |
| | | HSDAVFTEQY(OMe)TOrnLRAibQLAAAibOrnYLQAibIOrn |
| | | OrnGGPSSGAPPPCC-NH₂ |
| P516 | 60 | C6- |
| | | HSDAVFTEQY(OMe)TOrnLRAibQLAAbuAibOrnYLQAibIOrn |
| | | OrnGGPSSGAPPPCC-NH₂ |
| P518 | 61 | C6- |
| | | HSDAVFTEQY(OMe)TOrnLRAibQLAAAibOrnYAibQAibIOrn |
| | | OrnGGPSSGAPPPCC-NH₂ |
| P520 | 62 | C6- |
| | | HSDAVFTEQY(OMe)TOrnLRAibQLAAbuAibOrnYAibQSIOrn |
| | | OrnGGPSSGAPPPCC-NH₂ |
| P522 | 63 | C6- |
| | | HSDAVFTEQY(OMe)TOrnLRAibQLAAbuAibOrnYLQSIOrn |
| | | OrnGGPSSGAPPPCC-NH₂ |
| P524 | 64 | C6- |
| | | HSDAVFTEQY(OMe)TOrnLRAibQLAAbuAibOrnYAibQAibI |
| | | OrnOrnGGPSSGAPPPCC-NH₂ |
| P526 | 65 | C6- |
| | | HSDAVFTEQY(OMe)TOrnLRAibQLAAAibOrnYAibQSIOrn |
| | | OrnGGPSSGAPPPCC-NH₂ |
| P528 | 66 | C6- |
| | | HSDAVFTEQY(OMe)TOrnLRK(W)QVAAAibOrnYLQSIOrn |
| | | OrnGGPSSGAPPPS-NH₂ |
| P530 | 67 | C6- |
| | | HSDAVFTEQY(OMe)TOrnLRAibQVAAAibOrnYLK(W)SIOrn |
| | | OrnGGPSSGAPPPS-NH₂ |
| P532 | 68 | C6- |
| | | HSDAVFTEQY(OMe)TOrnLRAibQK(W)AAAibOrnYLQSIOrn |
| | | OrnGGPSSGAPPPS-NH₂ |
| P534 | 69 | C6- |
| | | HSDAVFTEQY(OMe)TOrnLRAibQVAAAibOrnYLQSK(W)Orn |
| | | OrnGGPSSGAPPPS-NH₂ |
| P536 | 70 | C6- |
| | | HSDAVFTEQY(OMe)TOrnLRAibQLAAbuAibOrnYLQAibIOrn |
| | | COrnGGPSSGAPPPS-NH₂ |
| P540 | 71 | C6- |
| | | HSDAVFTEQY(OMe)TOrnLRAibQLAAbuAibOrnYLQAibC |
| | | OrnOrnGGPSSGAPPPS-NH₂ |
| P544 | 72 | C6- |
| | | HSDAVFTEQY(OMe)TOrnLRAibQCAAbuAibOrnYLQAibIOrn |
| | | OrnGGPSSGAPPPS-NH₂ |
| P546 | 73 | C6- |
| | | HSDAVFTEQY(OMe)TOrnLRCQLAAbuAibOrnYLQAibIOrn |
| | | OrnGGPSSGAPPPS-NH₂ |
| P488 | 74 | C6- |
| | | HSDAVFTEQY(OMe)TOrnLRAibQVAAAibOrnYLQSIOrnOrn |
| | | GGPSSGAPPPS-NH₂ |
| P489 | 75 | C6-HSDAVFTEQY(OMe)TOrnLRAibQVAAAibOrnYL |
| | | K(CO(CH₂)₂SH)SIOrnOrnGGPSSGAPPPC-NH₂ |
| P491 | 76 | C6- |
| | | HSDAVFTEQY(OMe)TOrnLRAibQVAK(CO(CH₂)₂SH)AibOrn |
| | | YLQSIOrnOrnGGPSSGAPPPC-NH₂ |
| P494 | 77 | C6-HSDAVFTEQY(OMe)TOrnLRAibQVAAAibOrnYLQ |
| | | K(CO(CH₂)₂SH)IOrnOrnGGPSSGAPPPC-NH₂ |
| P496 | 78 | C6-HSDAVFTEQY(OMe)TOrnLRAibQVAAAibOrnYLQS |
| | | K(CO(CH₂)₂SH)OrnOrnGGPSSGAPPPC-NH₂ |
| P498 | 79 | C6-HSDAVFTEQY(OMe)TOrnLRAibQVAAAibOrnYLQSIOrn |
| | | K(CO(CH₂)₂SH)OrnGGPSSGAPPPC-NH₂ |
| P500 | 80 | C6- |
| | | HSDAVFTEQY(OMe)TOrnLRAibQVAAAibOrnYLCSIOrnOrn |
| | | GGPSSGAPPPC-NH₂ |
| P502 | 81 | C6- |
| | | HSDAVFTEQY(OMe)TOrnLRAibQVAAAibOrnYLQSCOrnOrn |
| | | GGPSSGAPPPC-NH₂ |
| P504 | 82 | C6- |
| | | HSDAVFTEQY(OMe)TOrnLRAibQVAAAibOrnYLQSIOrnC |
| | | OrnGGPSSGAPPPC-NH₂ |
| P479 | 111 | C6- |
| | | HSDAVFTEQY(OMe)TOrnLRAibQVK(CO(CH₂)₂SH)AAibOrn |
| | | YLQSIOrnOrnGGPSSGAPPPS-NH₂ |
| P538 | 112 | C6- |
| | | HSDAVFTEQY(OMe)TOrnLRAibQLAAbuAibOrnYLQSIOrnC |
| | | OrnGGPSSGAPPPS-NH₂ |
| P542 | 113 | C6- |
| | | HSDAVFTEQY(OMe)TOrnLRAibQLAAbuAibOrnYLQSCOrn |
| | | OrnGGPSSGAPPPS-NH₂ |
| P548 | 114 | C6- |
| | | HSDAVFTEQY(OMe)TOrnLRAibQLAAbuAibOrnYLQAibIOrn |
| | | K(CO(CH₂)₂SH)OrnGGPSSGAPPPC-NH₂ |
| P550 | 115 | C6- |
| | | HSDAVFTEQY(OMe)TOrnLRAibQLAAbuAibOrnYLQSIOrn |
| | | K(CO(CH₂)₂SH)OrnGGPSSGAPPPC-NH₂ |
| P554 | 116 | C6- |
| | | HSDAVFTEQY(OMe)TOrnLRAibQLAAbuAibOrnYLQSIOrnC |
| | | OrnGGPSSGAPPPC-NH₂ |
| P556 | 117 | C6- |
| | | HSDAVFTEQY(OMe)TOrnLRK(W)QLAAbuAibOrnYLQAibI |
| | | OrnOrnGGPSSGAPPPS-NH₂ |
| P559 | 118 | C6- |
| | | HSDAVFTEQY(OMe)TOrnLRAibQLAAAibOrnYLQSIOrnOrn |
| | | CGGPSSGAPPPS-NH₂ |
| P561 | 119 | C6- |
| | | HSDAVFTEQY(OMe)TOrnLRAibQVAAAibOrnYLQSIOrnOrn |
| | | CGGPSSGAPPPC-NH₂ |
| P563 | 120 | C6- |
| | | HSDAVFTEQY(OMe)TOrnLRAibQLAAbuAibOrnYLQAibIOrn |
| | | OrnCGGPSSGAPPPS-NH₂ |
| P565 | 121 | C6- |
| | | HSDAVFTEQY(OMe)TOrnLRAibQLAAbAibOrnYLQSIOrn |
| | | OrnCGGPSSGAPPPS-NH₂ |
| P571 | 122 | C6- |
| | | HSDAVFTEQY(OMe)TOrnLRAibQLAAbuAibOrnYLQSIOrn |
| | | OrnCGGPSSGAPPPC-NH₂ |
| P573 | 123 | C6- |
| | | HSDAVFTEQY(OMe)TOrnLRAibQLAAbuAibOrnY(OMe)LQ |
| | | AibIOrnOrnGGPSSGAPPPCC-NH₂ |
| P575 | 124 | C6- |
| | | HSDAVFTEQY(OMe)TOrnLRAibQLAAbuAibOrnY(OMe)LQ |
| | | AibIOrnCOrnGGPSSGAPPPS-NH₂ |
| P577 | 125 | C6- |
| | | HSDAVFTEQY(OMe)TOrnLRAibQCAAbuAibOrnY(OMe)LQ |
| | | AibIOrnOrnGGPSSGAPPPS-NH₂ |
| P579 | 126 | C6- |
| | | HSDAVFTEQY(OMe)TOrnLRAibQLAAbuAibOrnYLQAibIOrn |
| | | OrnCGGPSSGAPPPC-NH₂ |
| P581 | 127 | C6- |
| | | HSDAVFTEQY(OMe)TOrnLRAibQLAAbuAibOrnY(OMe)LQS |
| | | IOrnOrnGGPSSGAPPPCC-NH₂ |
| P583 | 128 | C6- |
| | | HSDAVFTEQY(OMe)TOrnLRAibQLAAbuAibOrnY(OMe)LQS |
| | | IOrnCOrnGGPSSGAPPPS-NH₂ |
| P585 | 129 | C6- |
| | | HSDAVFTEQY(OMe)TOrnLRAibQCAAbuAibOrnY(OMe)LQS |
| | | IOrnOrnGGPSSGAPPPS-NH₂ |
| P587 | 130 | C6- |
| | | HSDAVFTEQY(OMe)TOrnLRAibQLAAbuAibOrnY(OMe)LQS |
| | | IOrnCOrnGGPSSGAPPPC-NH₂ |
| P589 | 131 | C6- |
| | | HSDAVFTEQY(OMe)TOrnLRAibQLAbuAAibOrnYLQSIOrn |
| | | OrnGGPSSGAPPPCC-NH₂ |
| P596 | 132 | C6- |
| | | HSDAVFTEQY(OMe)TOrnLRAibQK(CO(CH₂)₂SH)AAbuAib |
| | | OrnYLQAibIOrnOrnGGPSSGAPPPC-NH₂ |
| P598 | 133 | C6- |
| | | HSDAVFTEQY(OMe)TOrnLRAibQK(CO(CH₂)₂SH)AAbuAib |
| | | OrnYLQAibIOrnOrnGGPSSGAPPPS-NH₂ |
| P600 | 134 | C6- |
| | | HSDAVFTEQY(OMe)TOrnLRAibQK(W)AAbuAibOrnYLQAibI |
| | | OrnOrnGGPSSGAPPPS-NH₂ |
| P484 | 136 | C6- |
| | | HSDAVFTEQY(OMe)TOrnLRK(CO(CH₂)₂SH)QVAAAibOrnY |
| | | LQSIOrnOrnGGPSSGAPPPS-NH₂ |
| P552 | 137 | C6 |
| | | HSDAVFTEQY(OMe)TOrnLRAibQLAAbuAibOrnYLQAibIOrn |
| | | COrnGGPSSGAPPPC-NH₂ |
| P567 | 138 | C6- |
| | | HSDAVFTEQY(OMe)TOrnLRAibQCAAAibOrnYLQSIOrnOrn |
| | | GGPSSGAPPPC-NH₂ |
| P591 | 139 | C6- |
| | | HSDAVFTEQY(OMe)TOrnLRAibQLAAbuAibOrnYAibQAibI |
| | | OrnOrnGGPSSGAPPPS-NH₂ |
| P594 | 141 | C6- |
| | | HSDAVFTEQY(OMe)TOrnLRAibQCAAbuAibOrnYLQAibIOrn |
| | | OrnGGPSSGAPPPC-NH₂ |
| P29 | 200 | HSDAVFTDNYTRLRKQVAAKKYLQSIKNKRQGGPSSGAPP |
| | | PS |
| P343 | 201 | C6- |
| | | HSDAVFTDNYThRLRAibQVAAAibKYLQSIKNKRYGGPSSG |
| | | APPPS |
| P357 | 202 | C6- |
| | | HSDAVFTDNYTRLRAibQVAAAibKYLQSIKAibOrnGGPSSG |
| | | APPPS |
| P358 | 203 | C6- |
| | | HSDAVFTDNY(OMe)TRLRAibQVAAAibKYLQSIKNKRYGG |
| | | PSSGAPPPS |
| P362 | 204 | C6- |
| | | HSEAVFTENYTOrnLRAibQVAAAibKYLQSIOrnNOrnGGPSS |
| | | GAPPPS |
| P363 | 205 | C6- |
| | | HSDAVFTDQYTOrnLRAibQVAAAibKYLQSIOrnQOrnGGPSS |
| | | GAPPPS |
| P367 | 206 | C6- |
| | | HSDAVFTDNYTLLRAibQVAAAibKYLQSIOrnNOrnGGPSSG |
| | | APPPS |
| P368 | 207 | C6- |
| | | HSDAVFTDNYTQLRAibQVAAAibKYLQSIOrnNOrnGGPSSG |
| | | APPPS |
| P369 | 208 | C6- |
| | | HSDAVFTDNYTFLRAibQVAAAibKYLQSIOrnNOrnGGPSSG |
| | | APPPS |
| P370 | 209 | C6- |
| | | HSDAVFTDNYTRLLAKLALQKYLQSIOrnNOrnGGPSSGAPP |
| | | PS |
| P371 | 210 | C6- |
| | | HSDAVFTDNYTOrnLLAKLALQKYLQSIOrnNOrnGGPSSGA |
| | | PPPS |
| P372 | 211 | C6- |
| | | HSDAVFTDNYTOrnLRAibQVAAAibKYLQCIOrnNOrnGGPS |
| | | SGAPPPS |
| P377 | 212 | C6- |
| | | HSDAVFTDNYTOrnLRAibQVAACOrnYLQSIOrnNOrnGGPSS |
| | | GAPPPS-NH₂ |
| P379 | 213 | C6- |
| | | HSEAVFTEQYTOrnLRAibQVAAAibOrnYLQSIOrnOrnGGPSS |
| | | GAPPPC-NH₂ |
| P382 | 214 | C6- |
| | | HSDAVFTDNYTOrnLRAibQVAAAibKYLQSSOrnNOrnGGPS |
| | | SGAPPPS |
| P383 | 215 | C6- |
| | | HSDAVFTDNYTOrnLRAibQVAAAibKYLSSIOrnNOrnGGPSS |
| | | GAPPPS |
| P384 | 216 | C6- |
| | | HSDAYFTDNYTOrnLRAibQVAAAibKYSQSIOrnNOrnGGPSS |
| | | GAPPPS |
| P385 | 217 | C6- |
| | | HSDAVFTDNYTOrnLRAibQVAAAibKSLQSIOrnNOrnGGPSS |
| | | GAPPPS |
| P386 | 218 | C6- |
| | | HSDAVFTDNYTOrnLRAibQVAAAibSYLQSIOrnNOrnGGPSS |
| | | GAPPPS |
| P387 | 219 | C6- |
| | | HSDAVFTDNYTOrnLRAibQVASAibKYLQSIOrnNOrnGGPSS |
| | | GAPPPS |
| P388 | 220 | C6- |
| | | HSDAVFTDNYTOrnLRAibQVSAAibKYLQSIOrnNOrnGGPSS |
| | | GAPPPS |
| P389 | 221 | C6- |
| | | HSDAVFTDNYTOrnLRAibQSAAAibKYLQSIOrnNOrnGGPSS |
| | | GAPPPS |
| P390 | 222 | C6- |
| | | HSDAVFTDNYTOrnLRAibSVAAAibKYLQSIOrnNOrnGGPSS |
| | | GAPPPS |
| P391 | 223 | C6- |
| | | HSDAVFTDNYTOrnSRAibQVAAAibKYLQSIOrnNOrnGGPSS |
| | | GAPPPS |
| P392 | 224 | C6- |
| | | HSDAVFTDSYTOrnLRAibQVAAAibKYLQSIOrnNOrnGGPSS |
| | | GAPPPS |
| P393 | 225 | C6- |
| | | HSEAVFTEQY(OMe)TOrnLRAibQLAAAibOrnYLQSIOrnOrn |
| | | GGPSSGAPPPS |
| P394 | 226 | C6- |
| | | HSDAVFTDQY(OMe)TOrnLRAibQLAAAibOrnYLQSIOrnOrn |
| | | GGPSSGAPPPS |
| P395 | 227 | C6- |
| | | HSDAVFTDQYTOrnLRAibQLAAAibOrnYLQSIOrnOrnGGPSS |
| | | GAPPPS |
| P396 | 228 | C6- |
| | | HSDAVFTDQYTOrnLRAibQVAAAibOrnYLQSIOrnOrnGGPS |
| | | SGAPPPS |
| P397 | 229 | C6- |
| | | HSDAVFTDNYTOrnLRAibQVAAAibOrnYLQSIOrnOrnGGPS |
| | | SGAPPPS |
| P398 | 230 | C6- |
| | | HSDAVFTEQY(OMe)TOrnLRAibQVAAAibOrnYLQSIOrnOrn |
| | | GGPSSGAPPPC-NH₂ |
| P405 | 231 | C6- |
| | | HSDAVFTDNYTRAibRAibQVAAAibKYLQSIKAibKGGPSSG |
| | | APPPS |
| P406 | 232 | C6- |
| | | HSDAVFTDQYTRAibRAibQVAAAibKYLQSIKAibKGGPSSG |
| | | APPPS |
| P407 | 233 | C6- |
| | | HSDAVFTDQYTRAibRAibQLAAAibKYLQSIKAibKGGPSSG |
| | | APPPS |
| P408 | 234 | C6- |
| | | HSDAVFTDQY(OMe)TRAibRAibQLAAAibKYLQSIKAibKGG |
| | | PSSGAPPPS |
| P409 | 235 | C6- |
| | | HSEAVFTEQY(OMe)TRAibRAibQLAAAibKYLQSIKAibKGG |
| | | PSSGAPPPS |
| P412 | 236 | C6- |
| | | HSDAVFTDNYTOrnLRK(W)QVAAAibKYLQSIOrnNOrnGGP |
| | | SSGAPPPS |
| P414 | 237 | C6- |
| | | HSDAVFTEQY(OMe)TOrnLRAibQLAAAibOrnY(OMe)LQSI |
| | | OrnOrnGGPSSGAPPPC-NH₂ |
| P418 | 238 | C6- |
| | | HSDAVFTEQY(OMe)TOrnLRAibQLAAAibOrnY(OMe)LQSI |
| | | OrnOrnGGPSSGAPPPS |
| P419 | 239 | C6- |
| | | HSDAVFTEQY(OMe)TOrnLRAibQVAAAibOrnYLQSCOrnOrn |
| | | GGPSSGAPPPS-NH₂ |
| P421 | 240 | C6-HSDAVFTEQY(OMe)TOrnLRAibQVAAAibOrnYLQS |
| | | K(CO(CH₂)₂SH)OrnOrnGGPSSGAPPPS-NH₂ |
| P423 | 241 | C6-HSDAVFTEQY(OMe)TOrnLRAibQVAAAibOrnYLQ |
| | | K(CO(CH₂)₂SH)IOrnOrnGGPSSGAPPPS-NH₂ |
| P425 | 242 | C6- |
| | | HSDAVFTEQY(Ome)TOrnLRAibQVAAAibOrnYLCSIOrnOrn |
| | | GGPSSGAPPPS-NH₂ |
| P427 | 243 | C6-HSDAVFTEQY(OMe)TOrnLRAibQVAAAibOrnYL |
| | | K(CO(CH₂)₂SH)SIOrnOrnGGPSSGAPPPS-NH₂ |
| P429 | 244 | C6- |
| | | HSDAVFTEQY(OMe)TOrnLRAibQVACAibOrnYLQSTOrnOrn |
| | | GGPSSGAPPPS-NH₂ |
| P431 | 245 | C6- |
| | | HSDAVFTEQY(OMe)TOrnLRAibQVAK(CO(CH₂)₂SH)AibOrn |
| | | YLQSIOrnOrnGGPSSGAPPPS-NH₂ |
| P433 | 246 | C6- |
| | | HSDAVFTEQY(OMe)TOrnLRAibQCAAAibOrnYLQSIOrnOrn |
| | | GGPSSGAPPPS-NH₂ |
| P435 | 247 | C6- |
| | | HSDAVFTEQY(OMe)TOrnLRAibQK(CO(CH₂)₂SH)AAAibOrn |
| | | YLQSIOrnOrnGGPSSGAPPPS-NH₂ |
| P437 | 248 | C6- |
| | | HSDAVFTEQY(OMe)TOrnLRAibCVAAAibOrnYLQSIOrnOrn |
| | | GGPSSGAPPPS-NH₂ |
| P439 | 249 | C6- |
| | | HSDAVFTEQY(OMe)TOrnLRAibK(CO(CH₂)₂SH)VAAAibOrn |
| | | YLQSIOrnOrnGGPSSGAPPPS-NH₂ |
| P442 | 250 | C6- |
| | | HSDAVFTEQY(OMe)TOrnLRAibQVAAAibOrnYLQSIOrnC |
| | | OrnGGPSSGAPPPS-NH₂ |
| P444 | 251 | C6-HSDAVFTEQY(OMe)TOrnLRAibQVAAAibOrnYLQSIOrn |
| | | K(CO(CH₂)₂SH)OrnGGPSSGAPPPS-NH₂ |
| P446 | 252 | C6- |
| | | HSDAVFTEQY(OMe)TOrnLRAibQVAAAibOrnYLQCIOrnOrn |
| | | GGPSSGAPPPC-NH₂ |
| P448 | 253 | C6- |
| | | HSDAVFTECY(OMe)TOrnLRAibQVAAAibOrnYLQSIOrnOrn |
| | | GGPSSGAPPPS-NH₂ |
| P455 | 254 | C6- |
| | | HSDAVFTEQY(OMe)TOrnCRAibQVAAAibOrnYLQSIOrnOrn |
| | | GGPSSGAPPPS-NH₂ |
| P457 | 255 | C6- |
| | | HSDAVFTEQY(OMe)TOrnK(CO(CH₂)₂SH)RAibQVAAAibOrn |
| | | YLQSIOrnOrnGGPSSGAPPPS-NH₂ |

It is more preferred that the VPAC2 receptor peptide agonist according to the first aspect of the present invention comprises an amino acid sequence selected from:

| | | |
|---|---|---|
| Agonist | SEQ ID | |
| # | NO | Sequence |

| | | |
|---|---|---|
| P416 | 47 | C6- |
| | | HSDAVFTEQY(OMe)TRAibRAibQLAAAibOrnY(OMe)LQSIK |
| | | AibOrnGGPSSGAPPPC-NH₂ |
| P487 | 55 | C6- |
| | | HSDAVFTEQY(OMe)TOrnLRAibQLAAAibOrnYLQSIOrnOrn |
| | | GGPSSGAPPPS-NH₂ |
| P520 | 62 | C6- |
| | | HSDAVFTEQY(OMe)TOrnLRAibQLAAbuAibOrnYAibQSIOrn |
| | | OrnGGPSSGAPPPCC-NH₂ |
| P524 | 64 | C6- |
| | | HSDAVFTEQY(OMe)TOrnLRAibQLAAbuAibOrnYAibQAibI |
| | | OrnOrnGGPSSGAPPPCC-NH₂ |
| P488 | 74 | C6- |
| | | HSDAVFTEQY(OMe)TOrnLRAibQYAAAibOrnYLQSIOrnOrn |
| | | GGPSSGAPPPS-NH₂ |
| P498 | 79 | C6-HSDAVFTEQY(OMe)TOrnLRAibQVAAAibOrnYLQSIOrn |
| | | K(CO(CH₂)₂SH)OrnGGPSSGAPPPC-NH₂ |
| P504 | 82 | C6- |
| | | HSDAVFTEQY(OMe)TOrnLRAibQVAAAibOrnYLQSIOrnC |
| | | OrnGGPSSGAPPPC-NH₂ |
| P538 | 112 | C6- |
| | | HSDAVFTEQY(OMe)TOrnLRAibQLAAbuAibOrnYLQSIOrnC |
| | | OrnGGPSSGAPPPS-NH₂ |
| P554 | 116 | C6- |
| | | HSDAVFTEQY(OMe)TOrnLRAibQLAAbuAibOrnYLQSIOrnC |
| | | OrnGGPSSGAPPPC-NH₂ |
| P561 | 119 | C6- |
| | | HSDAVFTEQY(OMe)TOrnLRAibQVAAAibOrnYLQSIOrnOrn |
| | | CGGPSSGAPPPC-NH₂ |
| P573 | 123 | C6- |
| | | HSDAVFTEQY(OMe)TOrnLRAibQLAAbuAibOrnY(OMe)LQ |
| | | AibIOrnOrnGGPSSGAPPPCC-NH₂ |
| P581 | 127 | C6- |
| | | HSDAVFTEQY(OMe)TOrnLRAibQLAAbuAibOrnY(OMe)LQSI |
| | | OrnOrnGGPSSGAPPPCC-NH₂ |
| P442 | 250 | C6- |
| | | HSDAVFTEQY(OMe)TOrnLRAibQVAAAibOrnYLQSIOrnC |
| | | OrnGGPSSGAPPPS-NH₂ |

According to a second aspect of the present invention, there is provided a VPAC2 receptor peptide agonist comprising an amino acid sequence of the formula:

Formula 4

(SEQ ID NO: 4)

Xaa₁-Xaa₂-Xaa₃-Xaa₄-Xaa₅-Xaa₆-Thr-Xaa₈-Xaa₉-Xaa₁₀-
Thr-Xaa₁₂-Xaa₁₃-Xaa₁₄-Xaa₁₅-Xaa₁₆-Xaa₁₇-Xaa₁₈-Abu-
Xaa₂₀-Xaa₂₁-Xaa₂₂-Xaa₂₃-Xaa₂₄-Xaa₂₅-Xaa₂₆-Xaa₂₇-
Xaa₂₈-Xaa₂₉-Xaa₃₀-Xaa₃₁-Xaa₃₂-Xaa₃₃-Xaa₃₄-Xaa₃₅-
Xaa₃₆-Xaa₃₇-Xaa₃₈-Xaa₃₉-Xaa₄₀

wherein:
Xaa₁ is: His, dH, or is absent;
Xaa₂ is: dA, Ser, Val, Gly, Thr, Leu, dS, Pro, or Aib;
Xaa₃ is: Asp or Glu;
Xaa₄ is: Ala, Ile, Tyr, Phe, Val, Thr, Leu, Trp, Gly, dA, Aib, or NMeA;
Xaa₅ is: Val, Leu, Phe, Ile, Thr, Trp, Tyr, dV, Aib, or NMeV;
Xaa₆ is: Phe, Ile, Leu, Thr, Val, Trp, or Tyr;
Xaa₈ is: Asp, Glu, Ala, Lys, Leu, Arg, or Tyr;
Xaa₉ is: Asn, Gln, Asp, Glu, Ser, Cys, Lys, or K(CO(CH₂)₂SH);
Xaa₁₀ is: Tyr, Trp, Tyr(OMe), Ser, Cys, or Lys;
Xaa₁₂ is: Arg, Lys, Glu, hR, Orn, Lys (isopropyl), Aib, Cit, Ala, Leu, Gln, Phe, Ser, or Cys;
Xaa₁₃ is: Leu, Phe, Glu, Ala, Aib, Ser, Cys, Lys, or K(CO(CH₂)₂SH);
Xaa₁₄ is: Arg, Leu, Lys, Ala, hR, Orn, Lys (isopropyl), Phe, Gln, Aib, Cit, Ser, or Cys;
Xaa₁₅ is: Lys, Ala, Arg, Glu, Leu, hR, Orn, Lys (isopropyl), Phe, Gln, Aib, K(Ac), Cit, Ser, Cys, K(W), or K(CO(CH₂)₂SH);
Xaa₁₆ is: Gln, Lys, Glu, Ala, hR, Orn, Lys (isopropyl), Cit, Ser, Cys, K(CO(CH₂)₂SH), or K(W);
Xaa₁₇ is: Val, Ala, Leu, Ile, Met, Nle, Lys, Aib, Ser, Cys, K(CO(CH₂)₂SH), or K(W);
Xaa₁₈ is: Ala, Ser, Cys, Lys, K(CO(CH₂)₂SH), K(W), Abu, or Nle;
Xaa₂₀ is: Lys, Gln, hR, Arg, Ser, His, Orn, Lys (isopropyl), Ala, Aib, Trp, Thr, Leu, Ile, Phe, Tyr, Val, K(Ac), Cit, Cys, K(CO(CH₂)₂SH), or K(W);
Xaa₂₁ is: Lys, His, Arg, Ala, Phe, Aib, Leu, Gln, Orn, hR, K(Ac), Cit, Ser, Cys, Val, Tyr, Ile, Thr, Trp, K(W), or K(CO(CH₂)₂SH);
Xaa₂₂ is: Tyr, Trp, Phe, Thr, Leu, Ile, Val, Tyr(OMe), Ala, Aib, Ser, Cys, Lys, K(W), or K(CO(CH₂)₂SH);
Xaa₂₃ is: Leu, Phe, Ile, Ala, Trp, Thr, Val, Aib, Ser, Cys, Lys, K(W), or K(CO(CH₂)₂SH);
Xaa₂₄ is: Gln, Glu, Asn, Ser, Cys, Lys, K(CO(CH₂)₂SH), or K(W);
Xaa₂₅ is: Ser, Asp, Phe, Ile, Leu, Thr, Val, Trp, Gln, Asn, Tyr, Aib, Glu, Cys, Lys, K(CO(CH₂)₂SH), or K(W);
Xaa₂₆ is: Ile, Leu, Thr, Val, Trp, Tyr, Phe, Aib, Ser, Cys, Lys, K(CO(CH₂)₂SH), or K(W);
Xaa₂₇ is: Lys, hR, Arg, Gln, Ala, Asp, Glu, Phe, Gly, His, Ile, Met, Asn, Pro, Ser, Thr, Val, Trp, Tyr, Lys (isopropyl), Cys, Leu, Orn, dK, K(W), or K(CO(CH₂)₂SH);
Xaa₂₈ is: Asn, Asp, Gln, Lys, Arg, Aib, Orn, hR, Cit, Pro, dK, Ser, Cys, K(CO(CH₂)₂SH), or K(W);
Xaa₂₉ is: Lys, Ser, Arg, Asn, hR, Ala, Asp, Glu, Phe, Gly, His, Ile, Leu, Met, Pro, Gln, Thr, Val, Trp, Tyr, Cys, Orn, Cit, Aib, K(W), K(CO(CH₂)₂SH), or is absent;
Xaa₃₀ is: Arg, Lys, Ile, Ala, Asp, Glu, Phe, Gly, His, Leu, Met, Asn, Pro, Gln, Ser, Thr, Val, Trp, Tyr, Cys, hR, Cit, Aib, Orn, K(W), K(CO(CH₂)₂SH), or is absent;
Xaa₃₁ is: Tyr, His, Phe, Thr, Cys, Ser, Lys, Gln, K(W), K(CO(CH₂)₂SH), or is absent;
Xaa₃₂ is: Ser, Cys, Lys, or is absent;
Xaa₃₃ is: Trp or is absent;
Xaa₃₄ is: Cys or is absent;
Xaa₃₅ is: Glu or is absent;
Xaa₃₆ is: Pro or is absent;
Xaa₃₇ is: Gly or is absent;
Xaa₃₈ is: Trp or is absent;
Xaa₃₉ is: Cys or is absent; and
Xaa₄₀ is: Arg or is absent
   wherein if Xaa₂₉, Xaa₃₀, Xaa₃₁, Xaa₃₂, Xaa₃₃, Xaa₃₄, Xaa₃₅, Xaa₃₆, Xaa₃₇, Xaa₃₈, or Xaa₃₉ is absent, the next amino acid present downstream is the next amino acid in the peptide agonist sequence,
   and a C-terminal extension wherein the N-terminus of the C-terminal extension is linked to the C-terminus of the peptide of Formula 4 and wherein the C-terminal extension comprises an amino acid sequence of the formula:

Formula 3

(SEQ ID NO: 3)

Xaa₁-Xaa₂-Xaa₃-Xaa₄-Xaa₅-Xaa₆-Xaa₇-Xaa₈-Xaa₉-
Xaa₁₀-Xaa₁₁-Xaa₁₂

wherein:
Xaa₁ is: Gly, Cys, or absent;
Xaa₂ is: Gly, Arg, or absent;
Xaa₃ is: Pro, Thr, or absent;
Xaa₄ is: Ser, or absent;
Xaa₅ is: Ser, or absent;
Xaa₆ is: Gly, or absent;
Xaa₇ is: Ala, or absent;
Xaa₈ is: Pro, or absent;
Xaa₉ is: Pro, or absent;
Xaa₁₀ is: Pro, or absent;
Xaa₁₁ is: Ser, Cys, or absent; and
Xaa₁₂ is: Cys, or absent;
   wherein at least five of Xaa₁ to Xaa₁₂ of the C-terminal extension are present and wherein if Xaa₁, Xaa₂, Xaa₃, Xaa₄, Xaa₅, Xaa₆, Xaa₇, Xaa₈, Xaa₉, Xaa₁₀, or Xaa₁₁ is absent, the next amino acid present downstream is the next amino acid in the C-terminal extension and wherein the C-terminal amino acid may be amidated.

Preferably, at least six of Xaa₁ to Xaa₁₂ of the C-terminal extension of Formula 3 is present. More preferably, seven, eight, nine, ten, eleven, or all of Xaa₁ to Xaa₁₂ of the C-terminal extension are present.

Preferably, the C-terminal extension of the VPAC2 receptor peptide agonist according to the second aspect of the present invention is selected from:

| | |
|---|---|
| SEQ ID NO: 5 | GGPSSGAPPPS |
| SEQ ID NO: 6 | GGPSSGAPPPS-NH₂ |
| SEQ ID NO: 7 | GGPSSGAPPPC |
| SEQ ID NO: 8 | GGPSSGAPPPC-NH₂ |
| SEQ ID NO: 9 | GRPSSGAPPPS |
| SEQ ID NO: 10 | GRPSSGAPPPS-NH₂ |
| SEQ ID NO: 11 | GGPSSGAPPPCC |
| SEQ ID NO: 12 | GGPSSGAPPPCC-NH₂ |

An alternative C-terminal extension according to the second aspect of the present invention may comprise an amino acid sequence of the formula:

Formula 13

(SEQ ID NO: 13)

Xaa₁-Xaa₂-Xaa₃-Xaa₄-Xaa₅-Xaa₆-Xaa₇-Xaa₈-Xaa₉-Xaa₁₀

wherein:
Xaa₁ is: Ser, Cys, Lys, K(W), K(CO(CH₂)₂SH), or absent;
Xaa₂ is: Arg, Ser, hR, Orn, His, Cys, Lys, K(W), K(CO(CH₂)₂SH), or absent;
Xaa₃ is: Thr, Cys, Lys, K(W), K(CO(CH₂)₂SH), or absent;
Xaa₄ is: Ser, Cys, Lys, K(W), K(CO(CH₂)₂SH), or absent;
Xaa₅ is: Pro, Ser, Ala, Cys, Lys, K(W), K(CO(CH₂)₂SH), or absent;
Xaa₆ is: Pro, Ser, Ala, Arg, Cys, Lys, K(W), K(CO(CH₂)₂SH), or absent;
Xaa₇ is: Pro, Ser, Ala, Cys, Lys, K(W), K(CO(CH₂)₂SH), or absent;
Xaa₈ is: Lys, K(W), Pro, Cys, K(CO(CH₂)₂SH), or absent;
Xaa₉ is: K(E-C₁₆), Ser, Cys, Lys, K(W), K(CO(CH₂)₂SH), or absent; and
Xaa₁₀ is: Ser, Cys, Lys, K(W), K(CO(CH₂)₂SH), or absent.

It is preferred that if Xaa₁, Xaa₂, Xaa₃, Xaa₄, Xaa₅, Xaa₆, Xaa₇, Xaa₈ or Xaa₉ of Formula 13 is absent, the next amino acid downstream is the next amino acid in the C-terminal extension. The C-terminal amino acid may be amidated.

Preferably, at least one of Xaa₁ to Xaa₁₀ of the C-terminal extension of Formula 13 is present. More preferably, at least two, three, four, five, six, seven, eight, nine or all of Xaa₁ to Xaa₁₀ of the C-terminal extension are present.

More preferably the alternative C-terminal extension of the VPAC2 receptor peptide agonist is selected from:

| | |
|---|---|
| SEQ ID NO: 83 | SRTSPPP |
| SEQ ID NO: 84 | SRTSPPP-NH₂ |
| SEQ ID NO: 85 | SSTSPRPPSS |
| SEQ ID NO: 86 | SSTSPRPPSS-NH₂ |
| SEQ ID NO: 87 | SRTSPPPK(W) |
| SEQ ID NO: 88 | SRTSPPPK(W)-NH₂ |
| SEQ ID NO: 89 | SRTSPPPC |
| SEQ ID NO: 90 | SRTSPPPC-NH₂ |

Preferably, the VPAC2 receptor peptide agonist according to the second aspect of the present invention comprises a sequence of the Formula 4 (SEQ ID NO: 4) wherein Xaa₃ is Asp or Glu, Xaa₈ is Asp or Glu, Xaa₉ is Asn or Gln, Xaa₁₀ is Tyr or Tyr(OMe), Xaa₁₂ is Arg, hR, Lys, or Orn, Xaa₁₄ is Arg, Gln, Aib, hR, Orn, Cit, Lys, Ala, or Leu, Xaa₁₅ is Lys, Aib, Orn, or Arg, Xaa₁₆ is Gin or Lys, Xaa₁₇ is Val, Leu, Ala, Ile, Lys, or Nle, Xaa₂₀ is Lys, Val, Leu, Aib, Ala, Gln, or Arg, Xaa₂₁ is Lys, Aib, Orn, Ala, Gln, or Arg, Xaa₂₃ is Leu or Aib, Xaa₂₅ is Ser or Aib, Xaa₂₇ is Lys, Orn, hR, or Arg, Xaa₂₈ is Asn, Gln, Lys, hR, Aib, Orn, or Pro and Xaa₂₉ is Lys, Orn, hR, or is absent.

Preferably, the VPAC2 receptor peptide according to the second aspect of the present invention comprises a sequence of the Formula 4 (SEQ ID NO: 4) wherein either Xaa₂₃ or Xaa₂₅ is Aib. Even more preferably, Xaa₂₃ and Xaa₂₅ are both Aib.

Preferably, the VPAC2 receptor peptide agonist according to the second aspect of the present invention comprises a sequence of the Formula 4 wherein either Xaa₁₄ or Xaa₁₅ is Aib.

Alternatively, the VPAC2 receptor peptide agonist according to the second aspect of the present invention comprises a sequence of the Formula 4 wherein either Xaa₂₀ or Xaa₂₁ is Aib.

More preferably, either Xaa₁₄ or Xaa₁₅ is Aib and either Xaa₂₀ or Xaa₂₀ is Aib. It is especially preferred that Xaa₁₅ is Aib and Xaa₂₀ is Aib.

Preferably, the VPAC2 receptor peptide agonist according to the second aspect of the present invention comprises a sequence of the Formula 4 wherein Xaa₁₅ is Aib, Xaa₂₀ is Aib, and Xaa₁₂, Xaa₂₁, Xaa₂₇ and Xaa₂₈ are all Orn. More preferably, Xaa₁₅ is Aib, Xaa₂₀ is Aib, Xaa₁₂, Xaa₂₁, Xaa₂₇ and Xaa₂₈ are all Orn, Xaa₈ is Glu, Xaa₉ is Gln and Xaa₁₀ is Tyr(OMe). Even more preferably, Xaa₁₅ is Aib, Xaa₂₀ is Aib, Xaa₁₂, Xaa₂₁, Xaa₂₇ and Xaa₂₈ are all Orn, Xaa₈ is Glu, Xaa₉ is Gln, Xaa₁₀ is Tyr(OMe), and Xaa₂₃ and/or Xaa₂₅ is Aib.

Preferably, the VPAC2 receptor peptide agonist according to the second aspect of the present invention further comprises a N-terminal modification at the N-terminus of the peptide agonist wherein the N-terminal modification is selected from:
(a) addition of D-histidine, isoleucine, methionine, or norleucine;
(b) addition of a peptide comprising the sequence Ser-Trp-Cys-Glu-Pro-Gly-Trp-Cys-Arg (SEQ ID NO: 91) wherein the Arg is linked to the N-terminus of the peptide agonist;
(c) addition of C₁-C₁₆ alkyl optionally substituted with one or more substituents independently selected from aryl, C₁-C₆ alkoxy, —NH₂, —OH, halogen and —CF₃;
(d) addition of —C(O)R¹ wherein R¹ is a C₁-C₁₆ alkyl optionally substituted with one or more substituents independently selected from aryl, C₁-C₆ alkoxy, —NH₂, —OH, halogen, —SH and —CF₃; an aryl optionally substituted with one or more substituents independently selected from C₁-C₆ alkyl, C₂-C₆ alkenyl, C₂-C₆ alkynyl, C₁-C₆ alkoxy, —NH₂, —OH, halogen and —CF₃; an aryl C₁-C₄ alkyl optionally substituted with one or more substituents independently selected from C₁-C₆ alkyl, C₂-C₆ alkenyl, C₂-C₆ alkynyl, C₁-C₆ alkoxy, —NH₂, —OH, halogen and —CF₃; —NR²R³ wherein R² and R³ are independently hydrogen, C₁-C₆ alkyl, aryl or aryl C₁-C₄ alkyl; —OR⁴ wherein R⁴ is C₁-C₁₆ alkyl optionally substituted with one or more substituents independently selected from aryl, C₁-C₆ alkoxy, —NH₂, —OH, halogen and —CF₃, aryl optionally substituted with one or more substituents independently selected from C₁-C₆ alkyl, C₂-C₆ alkenyl, C₂-C₆ alkynyl, C₁-C₆ alkoxy, —NH₂, —OH, halogen and —CF₃, or aryl C₁-C₄ alkyl optionally substituted with one or more substituents independently selected from C₁-C₆ alkyl, C₂-C₆ alkenyl, C₂-C₆ alkynyl, C₁-C₆ alkoxy, —NH₂, —OH, halogen and —CF₃; or 5-pyrrolidin-2-one;
(e) addition of —SO₂R⁵ wherein R⁵ is aryl, aryl C₁-C₄ alkyl or C₁-C₁₆ alkyl;
(f) formation of a succinimide group optionally substituted with C₁-C₆ alkyl or —SR⁶, wherein R⁶ is hydrogen or C₁-C₆ alkyl;
(g) addition of methionine sulfoxide;
(h) addition of biotinyl-6-aminohexanoic acid (6-aminocaproic acid); and
(i) addition of —C(═NH)—NH₂.

Preferably, the N-terminal modification is the addition of a group selected from: acetyl, propionyl, butyryl, pentanoyl, hexanoyl, methionine, methionine sulfoxide, 3-phenylpropionyl, phenylacetyl, benzoyl, norleucine, D-histidine, isoleucine, 3-mercaptopropionyl, biotinyl-6-aminohexanoic acid (6-aminocaproic acid), and —C(═NH)—NH₂. It is especially preferred that the N-terminal modification is the addition of acetyl or hexanoyl.

It will be appreciated by the person skilled in the art that VPAC2 receptor peptide agonists comprising various combinations of peptide sequence according to Formula 4, C-terminal extensions and N-terminal modifications as described herein, may be made based on the above disclosure.

It is preferred that the VPAC2 receptor peptide agonist according to the second aspect of the present invention comprises an amino acid sequence selected from:

| | | |
|---|---|---|
| Agonist | SEQ ID | |
| # | NO | Sequence |

| | | |
|---|---|---|
| P510 | 58 | C6- |
| | | HSDAVFTEQY(OMe)TOrnLRAibQVAAbuAibOrnYLQAibIOrn |
| | | OrnGGPSSGAPPPC-NH₂ |
| P516 | 60 | C6- |
| | | HSDAVFTEQY(OMe)TOrnLRAibQLAAbuAibOrnYLQAibIOrn |
| | | OrnGGPSSGAPPPCC-NH₂ |
| P520 | 62 | C6- |
| | | HSDAVFTEQY(OMe)TOrnLRAibQLAAbuAibOrnYAibQSIOrn |
| | | OrnGGPSSGAPPPCC-NH₂ |
| P522 | 63 | C6- |
| | | HSDAVFTEQY(OMe)TOrnLRAibQLAAbuAibOrnYLQSIOrn |
| | | OrnGGPSSGAPPPCC-NH₂ |
| P524 | 64 | C6- |
| | | HSDAVFTEQY(OMe)TOrnLRAibQLAAbuAibOrnYAibQAibI |
| | | OrnOrnGGPSSGAPPPCC-NH₂ |
| P536 | 70 | C6- |
| | | HSDAVFTEQY(OMe)TOrnLRAibQLAAbuAibOrnYLQAibIOrn |
| | | COrnGGPSSGAPPPS-NH₂ |
| P540 | 71 | C6- |
| | | HSDAVFTEQY(OMe)TOrnLRAibQLAAbuAibOrnYLQAibC |
| | | OrnOrnGGPSSGAPPPS-NH₂ |
| P544 | 72 | C6- |
| | | HSDAVFTEQY(OMe)TOrnLRAibQCAAbuAibOrnYLQAibIOrn |
| | | OrnGGPSSGAPPPS-NH₂ |
| P546 | 73 | C6- |
| | | HSDAVFTEQY(OMe)TOrnLRCQLAAbuAibOrnYLQAibIOrn |
| | | OrnGGPSSGAPPPS-NH₂ |
| P538 | 112 | C6- |
| | | HSDAVFTEQY(OMe)TOrnLRAibQLAAbuAibOrnYLQSIOrnC |
| | | OrnGGPSSGAPPPS-NH₂ |
| P542 | 113 | C6- |
| | | HSDAVFTEQY(OMe)TOrnLRAibQLAAbuAibOrnYLQSCOrn |
| | | OrnGGPSSGAPPPS-NH₂ |
| P548 | 114 | C6- |
| | | HSDAVFVEQY(OMe)TOrnLRAibQLAAbuAibOrnYLQAibIOrn |
| | | K(CO(CH₂)₂SH)OrnGGPSSGAPPPC-NH₂ |
| P550 | 115 | C6- |
| | | HSDAVFTEQY(OMe)TOrnLRAibQLAAbuAibOrnYLQSIOrn |
| | | K(CO(CH₂)₂SH)OrnGGPSSGAPPPC-NH₂ |
| P554 | 116 | C6- |
| | | HSDAVFTEQY(OMe)TOrnLRAibQLAAbuAibOrnYLQSIOrnC |
| | | OrnGGPSSGAPPPC-NH₂ |
| P556 | 117 | C6- |
| | | HSDAVFTEQY(OMe)TOrnLRK(W)QLAAbuAibOrnYLQAibI |
| | | OrnOrnGGPSSGAPPPS-NH₂ |
| P563 | 120 | C6- |
| | | HSDAVFTEQY(OMe)TOrnLRAibQLAAbuAibOrnYLQAibIOrn |
| | | OrnCGGPSSGAPPPS-NH₂ |
| P565 | 121 | C6- |
| | | HSDAVFTEQY(OMe)TOrnLRAibQLAAbuAibOrnYLQSIOrn |
| | | OrnCGGPSSGAPPPS-NH₂ |
| P571 | 122 | C6- |
| | | HSDAVFTEQY(OMe)TOrnLRAibQLAAbuAibOrnYLQSIOrn |
| | | OrnCGGPSSGAPPPC-NH₂ |
| P573 | 123 | C6- |
| | | HSDAVFTEQY(OMe)TOrnLRAibQLAAbuAibOrnY(OMe)LQ |
| | | AibIOrnOrnGGPSSGAPPPCC-NH₂ |
| P575 | 124 | C6- |
| | | HSDAVFTEQY(OMe)TOrnLRAibQLAAbuAibOrnY(OMe)LQ |
| | | AibIOrnCOrnGGPSSGAPPPS-NH₂ |
| P577 | 125 | C6- |
| | | HSDAVFTEQY(OMe)TOrnLRAibQCAAbuAibOrnY(OMe)LQ |
| | | AibIOrnOrnGGPSSGAPPPS-NH₂ |
| P579 | 126 | C6- |
| | | HSDAVFTEQY(OMe)TOrnLRAibQLAAbuAibOrnYLQAibIOrn |
| | | OrnCGGPSSGAPPPC-NH₂ |
| P581 | 127 | C6- |
| | | HSDAVFTEQY(OMe)TOrnLRAibQLAAbuAibOrnY(OMe)LQS |
| | | IOrnOrnGGPSSGAPPPCC-NH₂ |
| P583 | 128 | C6- |
| | | HSDAVFTEQY(OMe)TOrnLRAibQLAAbuAibOrnY(OMe)LQS |
| | | IOrnCOrnGGPSSGAPPPS-NH₂ |
| P585 | 129 | C6- |
| | | HSDAVFTEQY(OMe)TOrnLRAibQCAAbuAibOrnY(OMe)LQS |
| | | IOrnOrnGGPSSGAPPPS-NH₂ |
| P587 | 130 | C6- |
| | | HSDAVFTEQY(OMe)TOrnLRAibQLAAbuAibOrnY(OMe)LQS |
| | | IOrnCOrnGGPSSGAPPPC-NH₂ |
| P596 | 132 | C6- |
| | | HSDAVFTEQY(OMe)TOrnLRAibQK(CO(CH₂)₂SH)AAbuAib |
| | | OrnYLQAibIOrnOrnGGPSSGAPPPC-NH₂ |
| P598 | 133 | C6- |
| | | HSDAVFTEQY(OMe)TOrnLRAibQK(CO(CH₂)₂SH)AAbuAib |
| | | OrnYLQAibIOrnOrnGGPSSGAPPPS-NH₂ |
| P600 | 134 | C6- |
| | | HSDAVFTEQY(OMe)TOrnLRAibQK(W)AAbuAibOrnYLQAibI |
| | | OrnOrnGGPSSGAPPPS-NH₂ |
| P552 | 137 | C6- |
| | | HSDAVFTEQY(OMe)TOrnLRAibQLAAbuAibOrnYLQAibIOrn |
| | | COrnGGPSSGAPPPC-NH₂ |
| P591 | 139 | C6- |
| | | HSDAVFTEQY(OMe)TOrnLRAibQLAAbuAibOrnYAibQAibI |
| | | OrnOrnGGPSSGAPPPS-NH₂ |
| P594 | 141 | C6- |
| | | HSDAVFTEQY(OMe)TOrnLRAibQCAAbuAibOrnYLQAibIOrn |
| | | OrnGGPSSGAPPPC-NH₂ |

According to a third aspect of the present invention, there is provided a pharmaceutical composition comprising a VPAC2 receptor peptide agonist of the present invention and one or more pharmaceutically acceptable diluents, carriers and excipients.

According to a fourth aspect of the present invention, there is provided a VPAC2 receptor peptide agonist of the present invention for use as a medicament.

According to a fifth aspect of the present invention, there is provided the use of a VPAC2 receptor peptide agonist of the present invention for the manufacture of a medicament for the treatment of non-insulin-dependent diabetes.

According to a further aspect of the present invention, there is provided the use of a VPAC2 receptor peptide agonist of the present invention for the manufacture of a medicament for the treatment of insulin-dependent diabetes.

The present invention provides a method of treating diabetes in a patient in need thereof comprising administering a VPAC2 receptor peptide agonist of the present invention, wherein the diabetes may be non-insulin dependent diabetes or may be insulin-dependent diabetes.

The present invention further provides a pharmaceutical composition containing a VPAC2 receptor peptide agonist of the present invention for treating non-insulin dependent diabetes or insulin-dependent diabetes.

According to an alternative embodiment of the present invention, there is provided a VPAC2 receptor peptide agonist comprising a sequence selected from:

SEQ ID NO: 17

HSDAVFTEQY(OMe)TRAibRAibQLAAAibOrnY(OMe)LQSIK
AibOrn;

SEQ ID NO: 18

HSDAVFTEK(CO(CH2)2SH)Y(OMe)TOrnLRAibQVAAAibOrn
YLQSIOrnOrn;

SEQ ID NO: 19

HSDAVFTEQY(OMe)TOrnLRAibQVAAAibOrnYLQSIOrnK(W)
Orn;

SEQ ID NO: 20

HSDAVFTEQY(OMe)TOrnLRAibQVAAAibK(CO(CH2)2SH)YLQ
SIOrnOrn;

SEQ ID NO: 21

HSDAVFTEQY(OMe)TOrnLRAibQVAAK(CO(CH2)2SH)OrnYLQ
SIOrnOrn;

SEQ ID NO: 22

HSDAVFTEQY(OMe)TOrnLRAibQVCAAibOrnYLQSIOrnOrn;

SEQ ID NO: 23

HSDAVFTEQY(OMe)TOrnLRCQVAAAibOrnYLQSIOrnOrn;

SEQ ID NO: 24

HSDAVFTEQY(OMe)TOrnLRAibQLAAAibOrnYLQSIOrnOrn;

SEQ ID NO: 25

HSDAVFTEQY(OMe)TOrnLRAibQVAAAibOrnYAibQSIOrnOrn;

SEQ ID NO: 26

HSDAVFTEQY(OMe)TOrnLRAibQVAAAibOrnYLQAibIOrnOrn;

SEQ ID NO: 27

HSDAVFTEQY(OMe)TOrnLRAibQVAAbuAibOrnYLQAibIOrnOrn;

SEQ ID NO: 28

HSDAVFTEQY(OMe)TOrnLRAibQLAAAibOrnYLQAibIOrnOrn;

SEQ ID NO: 29

HSDAVFTEQY(OMe)TOrnLRAibQLAAAibOrnYAibQAibIOrnOrn;

SEQ ID NO: 30

HSDAVFTEQY(OMe)TOrnLRAibQLAAbuAibOrnYAibQSIOrnOrn;

SEQ ID NO: 31

HSDAVFTEQY(OMe)TOrnLRAibQLAAbuAibOrnYLQSIOrnOrn;

SEQ ID NO: 32

HSDAVFTEQY(OMe)TOrnLRAibQLAAbuAibOrnYAibQAibIOrn
Orn;

SEQ ID NO: 33

HSDAVFTEQY(OMe)TOrnLRAibQLAAAibOrnYAibQSIOrnOrn;

SEQ ID NO: 34

HSDAVFTEQY(OMe)TOrnLRK(W)QVAAAibOrnYLQSIOrnOrn;

SEQ ID NO: 35

HSDAVFTEQY(OMe)TOrnLRAibQVAAAibOrnYLK(W)SIOrnOrn;

SEQ ID NO: 36

HSDAVFTEQY(OMe)TOrnLRAibQK(W)AAAibOrnYLQSIOrnOrn;

SEQ ID NO: 37

HSDAVFTEQY(OMe)TOrnLRK(CO(CH2)2SH)QVAAAibOrnYLQ
SIOrnOrnn:

SEQ ID NO: 38

HSDAVFTEQY(OMe)TOrnLRAibQVAAAibK(W)YLQSIOrnOrn;

SEQ ID NO: 39

HSDAVFTEQY(OMe)TOrnLRAibQVAAAibCYLQSIOrnOrn;

SEQ ID NO: 40

HSDAVFTEQY(OMe)TOrnLRAibQLAAbuAibOrnYLQAibIOrnOrn;

SEQ ID NO: 41

HSDAVFTEQY(OMe)TOrnLRAibQVAAAibOrnYLQSK(W)OrnOrn;

SEQ ID NO: 42

HSDAVFTEQY(OMe)TOrnLRAibQLAAbuAibOrnYLQAibIOrnC
Orn;

SEQ ID NO: 43

HSDAVFTEQY(OMe)TOrnLRAibQLAAbuAibOrnYLQAibCOrn
Orn;

SEQ ID NO: 44

HSDAVFTEQY(OMe)TOrnLRAibQCAAbuAibOrnYLQAibIOrnOrn;

SEQ ID NO: 45

HSDAVFTEQY(OMe)TOrnLRCQLAAbuAibOrnYLQAibIOrnOrn;

SEQ ID NO: 92

HSDAVFTEQY(OMe)TOrnLRAibQVK(CO(CH2)2SH)AAibOrn
YLQSIOrnOrn;

SEQ ID NO: 93

HSDAVFTEQY(OMe)TOrnLRAibQLAAbuAibOrnYLQSIOrnCOrn;

SEQ ID NO: 94

HSDAVFTEQY(OMe)TOrnLRAibQLAAbuAibOrnYLQSCOrnOrn;

SEQ ID NO: 95

HSDAVFTEQY(OMe)TOrnLRAibQLAAbuAibOrnYLQAibIOrn
K(CO(CH₂)₂SH)Orn;

SEQ ID NO: 96

HSDAVFTEQY(OMe)TOrnLRAibQLAAbuAibOrnYLQSIOrn
K(CO(CH₂)₂SH)Orn;

SEQ ID NO: 97

HSDAVFTEQY(OMe)TOrnLRK(W)QLAAbuAibOrnYLQAibIOrn
Orn;

SEQ ID NO: 98

HSDAVFTEQY(OMe)TOrnLRAibQLAAAibOrnYLQSIOrnOrnC;

SEQ ID NO: 99

HSDAVFTEQY(OMe)TOrnLRAibQVAAAibOrnYLQSIOrnOrnC;

SEQ ID NO: 100

HSDAVFTEQY(OMe)TOrnLRAibQLAAbuAibOrnYLQSIOrnOrnC;

SEQ ID NO: 101

HSDAVFTEQY(OMe)TOrnLRAibQLAAbuAibOrnY(OMe)LQAibI
OrnOrn;

SEQ ID NO: 102

HSDAVFTEQY(OMe)TOrnLRAibQLAAbuAibOrnY(OMe)LQAibI
OrnCOrn;

SEQ ID NO: 103

HSDAVFTEQY(OMe)TOrnLRAibQCAAbuAibOrnY(OMe)LQAibI
OrnOrn;

SEQ ID NO: 104

HSDAVFTEQY(OMe)TOrnLRAibQLAAbuAibOrnYLQAibIOrn
OrnC;

SEQ ID NO: 105

HSDAVFTEQY(OMe)TOrnLRAibQLAAbuAibOrnY(OMe)LQSI
OrnOrn;

SEQ ID NO: 106

HSDAVFTEQY(OMe)TOrnLRAibQCAAbuAibOrnY(OMe)LQSI
OrnOrn;

SEQ ID NO: 107

HSDAVFTEQY(OMe)TOrnLRAibQLAAbuAibOrnY(OMe)LQSI
OrnCOrn;

SEQ ID NO: 108

HSDAVFTEQY(OMe)TOrnLRAibQLAbuAAibOrnYLQSIOrnOrn;

SEQ ID NO: 109

HSDAVFTEQY(OMe)TOrnLRAibQK(CO(CH2)2SH)AAbu
AibOrnYLQAibIOrnOrn;

SEQ ID NO: 110

HSDAVFTEQY(OMe)TOrnLRAibQK(W)AAbuAibOrnYLQ
AibIOrnOrn;

SEQ ID NO: 145

HSDAVFTDNYTRLRKQVAAKKYLQSIKNKRQ;

SEQ ID NO: 146

HSDAVFTDNYTLLRAibQVAAAibKYLQSIOrnNOrn;

SEQ ID NO: 147

HSDAVFTDNYTQLRAibQVAAAibKYLQSIOrnNOrn;

SEQ ID NO: 148

HSDAVFTDNYTFLRAibQVAAAibKYLQSIOrnNOrn;

SEQ ID NO: 149

HSDAVFTDNYTOrnLRAibQVAAAibKYLQCIOrnNOrn;

SEQ ID NO: 150

HSDAVFTDNYTOrnLRAibQVAACOrnYLQSIOrnNOrn;

SEQ ID NO: 151

HSDAVFTDNYTOrnLRAibQVAAAibKYLQSSOrnNOrn;

SEQ ID NO: 152

HSDAVFTDNYTOrnLRAibQVAAAibKYLSSIOrnNOrn;

SEQ ID NO: 153

HSDAVFTDNYTOrnLRAibQVAAAibKYSQSIOrnNOrn;

SEQ ID NO: 154

HSDAVFTDNYTOrnLRAibQVAAAibKSLQSIOrnNOrn;

SEQ ID NO: 155

HSDAVFTDNYTOrnLRAibQVAAAibSYLQSIOrnNOrn;

SEQ ID NO: 156

HSDAVFTDNYTOrnLRAibQVSAAibKYLQSIOrnNOrn;

SEQ ID NO: 157

HSDAVFTDNYTOrnLRAibQSAAAibKYLQSIOrnNOrn;

SEQ ID NO: 158

HSDAVFTDNYTOrnLRAibSVAAAibKYLQSIOrnNOrn;

SEQ ID NO: 159

HSDAVFTDNYTOrnSRAibQVAAAibKYLQSIOrnNOrn;

SEQ ID NO: 160

HSDAVFTDSYTOrnLRAibQVAAAibKYLQSIOrnNOrn;

SEQ ID NO: 161

HSDAVFTDNYThRLRAibQVAAAibKYLQSIKNKRY;

SEQ ID NO: 162

HSDAVFTDNYTRLRAibQVAAAibKYLQSIKAibOrn;

SEQ ID NO: 163

HSDAVFTDNY(OMe)TRLRAibQVAAAibKYLQSIKNKRY;

SEQ ID NO: 164

HSEAVFTENYTOrnLRAibQVAAAibKYLQSIOrnNOrn;

SEQ ID NO: 165

HSDAVFTDQYTOrnLRAibQVAAAibKYLQSIOrnQOrn;

SEQ ID NO: 166

HSDAVFTDNYTRLLAKLALQKYLQSIOrnNOrn;

SEQ ID NO: 167

HSDAVFTDNYTOrnLLAKLALQKYLQSIOrnNOrn;

SEQ ID NO: 168

HSEAVETEQYTOrnLRAibQVAAAibOrnYLQSIOrnOrn;

SEQ ID NO: 169

HSDAVFTDNYTOrnLRAibQVASAibKYLQSIOrnNOrn;

SEQ ID NO: 170

HSEAVFTEQY(OMe)TOrnLRAibQLAAAibOrnYLQSIOrnOrn;

SEQ ID NO: 171

HSDAVFTDQY(OMe)TOrnLRAibQLAAAibOrnYLQSIOrnOrn;

SEQ ID NO: 172

HSDAVFTDQYTOrnLRAibQLAAAibOrnYLQSIOrnOrn;

SEQ ID NO: 173

HSDAVFTDQYTOrnLRAibQVAAAibOrnYLQSIOrnOrn;

SEQ ID NO: 174

HSDAVFTDNYTOrnLRAibQVAAAibOrnYLQSIOrnOrn;

SEQ ID NO: 175

HSDAVFTEQY(OMe)TOrnLRAibQVAAAibOrnYLQSIOrnOrn;

SEQ ID NO: 176

HSDAVFTDNYTRAibRAibQVAAAibKYLQSIKAibK;

SEQ ID NO: 177

HSDAVFTDQYTRAibRAibQVAAAibKYLQSIKAibK;

SEQ ID NO: 178

HSDAVFTDQYTRAibRAibQLAAAibKYLQSIKAibK;

SEQ ID NO: 179

HSDAVFTDQY(OMe)TRAibRAibQLAAAibKYLQSIKAibK;

SEQ ID NO: 180

HSEAVFTEQY(OMe)TRAibRAibQLAAAibKYLQSIKAibK;

SEQ ID NO: 181

HSDAVFTEQY(OMe)TOrnLRAibQVAAAibOrnYLCSIOrnOrn;

SEQ ID NO: 182

HSDAVFTEQY(OMe)TOrnLRAibQVAAAibOrnYL
K(CO(CH₂)₂SH)SIOrnOrn;

SEQ ID NO: 183

HSDAVFTEQY(OMe)TOrnLRAibQVACAibOrnYLQSIOrnOrn;

SEQ ID NO: 184

HSDAVFTEQY(OMe)TOrnLRAibQVAK(CO(CH2)2SH)AibOrn
YLQSIOrnOrn;

SEQ ID NO: 185

HSDAVFTEQY(OMe)TOrnLRAibCVAAAibOrnYLQSIOrnOrn;

SEQ ID NO: 186

HSDAVFTDNYTOrnLRK(W)QVAAAibKYLQSIOrnNOrn;

SEQ ID NO: 187

HSDAVFTEQY(OMe)TOrnLRAibQLAAAibOrnY(OMe)LQ
SIOrnOrn;

SEQ ID NO: 188

HSDAVFTEQY(OMe)TOrnLRAibQVAAAibOrnYLQSCOrnOrn;

SEQ ID NO: 189

HSDAVFTEQY(OMe)TOrnLRAibQCAAAibOrnYLQSIOrnOrn;

SEQ ID NO: 190

HSDAVFTEQY(OMe)TOrnLRAibQVAAAibOrnYLQSIOrnCOrn;

SEQ ID NO: 191

HSDAVFTEQY(OMe)TOrnLRAibQVAAAibOrnYLQCIOrnOrn;

SEQ ID NO: 192

HSDAVFTECY(OMe)TOrnLRAibQVAAAibOrnYLQSIOrnOrn;

SEQ ID NO: 193

HSDAVFTEQY(OMe)TOrnCRAibQVAAAibOrnYLQSIOrnOrn;

SEQ ID NO: 194

HSDAVFTEQY(OMe)TOrnLRAibQVAAAibOrnYLQS
K(CO(CH₂)₂SH)OrnOrn;

SEQ ID NO: 195

HSDAVFTEQY(OMe)TOrnLRAibQVAAAibOrnYLQ
K(CO(CH₂)₂SH)IOrnOrn;

SEQ ID NO: 196

HSDAVFTEQY(OMe)TOrnLRAibQK(CO(CH₂)₂SH)AAAibOrnYL
QSIOrnOrn;

SEQ ID NO: 197

HSDAVFTEQY(OMe)TOrnLRAibK(CO(CH₂)₂SH)VAAAibOrnY
LQSIOrnOrn;

SEQ ID NO: 198

HSDAVFTEQY(OMe)TOrnLRAibQVAAAibOrnYLQSIOrn
K(CO(CH₂)₂SH)Orn;
and

SEQ ID NO: 199

HSDAVFTEQY(OMe)TOrnK(CO(CH₂)₂SH)RAibQVAAAibOrn
YLQSIOrnOrn.

Preferably, the VPAC2 receptor peptide agonist of the above alternative embodiment further comprises a C-terminal extension, wherein the N-terminus of the C-terminal extension is linked to the C-terminus of the peptide sequence and wherein the C-terminal extension comprises an amino acid sequence of the formula:

Formula 1

(SEQ ID NO: 1)

Xaa₁-Xaa₂-Xaa₃-Xaa₄-Xaa₅-Xaa₆-Xaa₇-Xaa₈-Xaa₉-
Xaa₁₀-Xaa₁₁-Xaa₁₂-Xaa₁₃

wherein:
Xaa₁ is: Gly, Cys, Lys, K(W), K(CO(CH₂)₂SH), or absent;
Xaa₂ is: Gly, Arg, Cys, Lys, K(W), K(CO(CH₂)₂SH), or absent;
Xaa₃ is: Pro, Thr, Ser, Ala, Cys, Lys, K(W), K(CO(CH₂)₂SH), or absent;
Xaa₄ is: Ser, Pro, His, Cys, Lys, K(W), K(CO(CH₂)₂SH), or absent;
Xaa₅ is: Ser, Arg, Thr, Trp, Lys, Cys, K(W), K(CO(CH₂)₂SH), or absent;
Xaa₆ is: Gly, Ser, Cys, Lys, K(W), K(CO(CH₂)₂SH), or absent;
Xaa₇ is: Ala, Asp, Arg, Glu, Lys, Gly, Cys, K(W), K(CO(CH₂)₂SH), or absent;
Xaa₈ is: Pro, Ser, Ala, Cys, Lys, K(W), K(CO(CH₂)₂SH), or absent;
Xaa₉ is: Pro, Ser, Ala, Cys, Lys, K(W), K(CO(CH₂)₂SH), or absent;
Xaa₁₀ is: Pro, Ser, Ala, Arg, Lys, His, Cys, K(W), K(CO(CH₂)₂SH), or absent;
Xaa₁₁ is: Ser, Cys, His, Pro, Lys, Arg, K(W), K(CO(CH₂)₂SH), or absent;
Xaa₁₂ is: His, Ser, Arg, Lys, Cys, K(W), K(CO(CH₂)₂SH), or absent; and
Xaa₁₃ is: His, Ser, Arg, Lys, Cys, K(W), K(CO(CH₂)₂SH), or absent;

provided that if Xaa₁, Xaa₂, Xaa₃, Xaa₄, Xaa₅, Xaa₆, Xaa₇, Xaa₈, Xaa₉, Xaa₁₀, Xaa₁₁, or Xaa₁₂ is absent, the next amino acid present downstream is the next amino acid in the C-terminal extension and wherein the C-terminal amino acid may be amidated.

It is preferable that the C-terminal extension of formula 1 has no more than three of any one of the following; Cys, Lys, K(W) or K(CO(CH₂)₂SH). It is more preferable that the C-terminal extension has no more than two of any of these residues. If there are two Cys residues in the C-terminal extension, it is preferred that the Cys residues are at the C-terminus. It is even more preferable that the C-terminal extension has no more than one of any of these residues. If there is only one Cys residue in the C-terminal extension, it is preferred that the Cys residue is at the C-terminus.

Preferably, the C-terminal extension of the VPAC2 receptor peptide agonist according to the above alternative embodiment comprises an amino acid sequence of the formula:

Formula 2

(SEQ ID NO: 2)

Xaa₁-Xaa₂-Xaa₃-Xaa₄-Xaa₅-Xaa₆-Xaa₇-Xaa₈-Xaa₉-
Xaa₁₀-Xaa₁₁-Xaa₁₂-Xaa₁₃

wherein:
Xaa₁ is: Gly, Cys, Lys, or absent;
Xaa₂ is: Gly, Arg, Cys, Lys, or absent;
Xaa₃ is: Pro, Thr, Ser, Ala, Cys, Lys, or absent;
Xaa₄ is: Ser, Pro, His, Cys, Lys, or absent;
Xaa₅ is: Ser, Arg, Thr, Trp, Lys, Cys, or absent;
Xaa₆ is: Gly, Ser, Cys, Lys, or absent;
Xaa₇ is: Ala, Asp, Arg, Glu, Lys, Gly, Cys, or absent;
Xaa₈ is: Pro, Ser, Ala, Cys, Lys, or absent;
Xaa₉ is: Pro, Ser, Ala, Cys, Lys, or absent;
Xaa₁₀ is: Pro, Ser, Ala, Arg, Lys, His, Cys, or absent;
Xaa₁₁ is: Ser, Cys, His, Pro, Lys, Arg, or absent;
Xaa₁₂ is: His, Ser, Arg, Lys, Cys, or absent; and
Xaa₁₃ is: His, Ser, Arg, Lys, Cys, or absent;

provided that if Xaa₁, Xaa₂, Xaa₃, Xaa₄, Xaa₅, Xaa₆, Xaa₇, Xaa₈, Xaa₉, Xaa₁₀, Xaa₁₁, or Xaa₁₂ is absent, the next amino acid present downstream is the next amino acid in the C-terminal extension and wherein the C-terminal amino acid may be amidated.

Preferably, at least one of Xaa₁ to Xaa₁₃ of the C-terminal extension of Formula 1 or 2 is present. More preferably, at least two, three, four, five, six, seven, eight, nine, ten, eleven, twelve, or all of Xaa₁ to Xaa₁₃ of the C-terminal extension are present.

More preferably, the C-terminal extension of the VPAC2 receptor peptide agonist according to the above alternative embodiment comprises an amino acid sequence of the formula:

Formula 3

(SEQ ID NO: 3)

Xaa₁-Xaa₂-Xaa₃-Xaa₄-Xaa₅-Xaa₆-Xaa₇-Xaa₈-Xaa₉-
Xaa₁₀-Xaa₁₁-Xaa₁₂

wherein:
Xaa₁ is: Gly, Cys, or absent;
Xaa₂ is: Gly, Arg, or absent;
Xaa₃ is: Pro, Thr, or absent;
Xaa₄ is: Ser, or absent;
Xaa₅ is: Ser, or absent;
Xaa₆ is: Gly, or absent;
Xaa₇ is: Ala, or absent;
Xaa₈ is: Pro, or absent;
Xaa₉ is: Pro, or absent;
Xaa₁₀ is: Pro, or absent;
Xaa₁₁ is: Ser, Cys, or absent; and
Xaa₁₂ is: Cys, or absent;

provided that if Xaa₁, Xaa₂, Xaa₃, Xaa₄, Xaa₅, Xaa₆, Xaa₇, Xaa₈, Xaa₉, Xaa₁₀, or Xaa₁₁ is absent, the next amino acid present downstream is the next amino acid in the C-terminal extension and wherein the C-terminal amino acid may be amidated.

Preferably, at least one of Xaa₁ to Xaa₁₂ of the C-terminal extension of Formula 3 is present. More preferably, at least two, three, four, five, six, seven, eight, nine, ten, eleven, or all of Xaa₁ to Xaa₁₂ of the C-terminal extension are present.

An alternative C-terminal extension may comprise an amino acid sequence of the formula:

Formula 13

(SEQ ID NO: 13)

Xaa₁-Xaa₂-Xaa₃-Xaa₄-Xaa₅-Xaa₆-Xaa₇-Xaa₈-Xaa₉-Xaa₁₀

wherein:
Xaa₁ is: Ser, Cys, Lys, K(W), K(CO(CH₂)₂SH), or absent;
Xaa₂ is: Arg, Ser, hR, Orn, His, Cys, Lys, K(W), K(CO(CH₂)₂SH), or absent;
Xaa₃ is: Thr, Cys, Lys, K(W), K(CO(CH₂)₂SH), or absent;
Xaa₄ is: Ser, Cys, Lys, K(W), K(CO(CH₂)₂SH), or absent;
Xaa₅ is: Pro, Ser, Ala, Cys, Lys, K(W), K(CO(CH₂)₂SH), or absent;
Xaa₆ is: Pro, Ser, Ala, Arg, Cys, Lys, K(W), K(CO(CH₂)₂SH), or absent;
Xaa₇ is: Pro, Ser, Ala, Cys, Lys, K(W), K(CO(CH₂)₂SH), or absent;
Xaa₈ is: Lys, K(W), Pro, Cys, K(CO(CH₂)₂SH), or absent;
Xaa₉ is: K(E-C₁₆), Ser, Cys, Lys, K(W), K(CO(CH₂)₂SH), or absent; and
Xaa₁₀ is: Ser, Cys, Lys, K(W), K(CO(CH₂)₂SH), or absent.

It is preferred that if Xaa₁, Xaa₂, Xaa₃, Xaa₄, Xaa₅, Xaa₆, Xaa₇, Xaa₈ or Xaa₉ of Formula 13 is absent, the next amino acid downstream is the next amino acid in the C-terminal extension. The C-terminal amino acid may be amidated.

Preferably, at least one of Xaa₁ to Xaa₁₀ of the C-terminal extension of Formula 13 is present. More preferably, at least two, three, four, five, six, seven, eight, nine or all of Xaa₁ to Xaa₁₀ of the C-terminal extension are present.

More preferably, the alternative C-terminal extension of Formula 13 is selected from:

| | |
|---|---|
| SEQ ID NO: 83 | SRTSPPP |
| SEQ ID NO: 84 | SRTSPPP-NH₂ |
| SEQ ID NO: 85 | SSTSPRPPSS |
| SEQ ID NO: 86 | SSTSPRPPSS-NH₂ |
| SEQ ID NO: 87 | SRTSPPPK(W) |
| SEQ ID NO: 88 | SRTSPPPK(W)-NH₂ |
| SEQ ID NO: 89 | SRTSPPPC |
| SEQ ID NO: 90 | SRTSPPPC-NH₂ |

Alternative VPAC2 receptor peptide agonists include:

| Agonist | SEQ ID | |
|---|---|---|
| # | NO | Sequence |
| P18 | 256 | HSDAVFTENY(OMe)TKLRKQNleAAKKYLNDLKKGGPSSG |
| | | APPPS |
| P401 | 135 | C6- |
| | | HSDAVFTEQY(OMe)TOrnLRAibQVAAAibOrnYLQSIOrnOrn |
| | | GGPSSGAPPPK(W)-NH₂ |
| P592 | 140 | C6- |
| | | HSDAVFTEQY(OMe)TOrnLRAibQLAAbuAibOrnYAibQAibI |
| | | OrnOrnGGPSSGAPPPK(W)-NH₂ |
| P901 | 142 | Pyr- |
| | | HSDAVFTDNYTRLRAibQVAAAibKYLQSIKNKRYGGPSSG |
| | | APPPS |
| P902 | 143 | Aha- |
| | | HSDAVFTDNYTRLRAibQVAAAibKYLQSIKNKRYGGPSSG |
| | | APPPS |
| P903 | 144 | C6- |
| | | HSDAVFTDNYTRLRAibQVAAAibKYLQSIKQGRPSSGAPPP |
| | | S |

The VPAC2 receptor peptide agonists of the present invention have the advantage that they have enhanced selectivity, potency and/or stability over known VPAC2 receptor peptide agonists. In particular, the addition of the C-terminal sequence of Exendin-4 as the c-capping sequence surprisingly increased the VPAC2 receptor selectivity as well as increasing proteolytic stability.

The term “VPAC2” is used to refer to and in conjunction with the particular receptor (Lutz, et al., FEBS Lett., 458:197-203 (1999); Adamou, et al., Biochem. Biophys. Res. Commun., 209: 385-392 (1995)) that the agonists of the present invention activate. This term also is used to refer to and in conjunction with the agonists of the present invention.

A “selective VPAC2 receptor peptide agonist” or a “VPAC2 receptor peptide agonist” of the present invention is a peptide that selectively activates the VPAC2 receptor to induce insulin secretion. The sequence for a selective VPAC2 receptor peptide agonist of the present invention has twenty-eight to forty naturally occurring and/or non-naturally occurring amino acids and may or may not additionally comprise a C-terminal extension.

The “C-terminal extension” of the present invention comprises a sequence having from one to thirteen naturally occurring or non-naturally occurring amino acids linked to the C-terminus of the sequence at the N-terminus of the C-terminal extension via a peptide bond.

As used herein, the term “linked to” with reference to the term C-terminal extension, includes the addition or attachment of amino acids or chemical groups directly to the C-terminus of the peptide sequence.

Optionally, the selective VPAC2 receptor peptide agonist may also have an N-terminal modification. The term “N-terminal modification” as used herein includes the addition or attachment of amino acids or chemical groups directly to the N-terminus of a peptide and the formation of chemical groups, which incorporate the nitrogen at the N-terminal of a peptide.

The N-terminal modification may comprise the addition of one or more naturally occurring or non-naturally occurring amino acids to the VPAC2 receptor peptide agonist sequence, preferably there are not more than ten amino acids, with one amino acid being more preferred. Naturally occurring amino acids which may be added to the N-terminus include methionine and isoleucine. A modified amino acid added to the N-terminus may be D-histidine. Alternatively, the following amino acids may be added to the N-terminus: SEQ ID NO: 91 Ser-Trp-Cys-Glu-Pro-Gly-Trp-Cys-Arg, wherein the Arg is linked to the N-terminus of the peptide agonist. Preferably, any amino acids added to the N-terminus are linked to the N-terminus by a peptide bond.

The term “linked to” as used herein, with reference to the term N-terminal modification, includes the addition or attachment of amino acids or chemical groups directly to the N-terminus of the VPAC2 receptor agonist. The addition of the above N-terminal modifications may be achieved under normal coupling conditions for peptide bond formation.

The N-terminus of the peptide agonist may also be modified by the addition of an alkyl group (R), preferably a C₁-C₁₆ alkyl group, to form (R)NH—.

Alternatively, the N-terminus of the peptide agonist may be modified by the addition of a group of the formula —C(O)R¹ to form an amide of the formula R¹C(O)NH—. The addition of a group of the formula —C(O)R¹ may be achieved by reaction with an organic acid of the formula R¹COOH. Modification of the N-terminus of an amino acid sequence using acylation is demonstrated in the art (e.g. Gozes et al., J. Pharmacol Exp Ther, 273:161-167 (1995)). Addition of a group of the formula —C(O)R¹ may result in the formation of a urea group (see WO 01/23240, WO 04/06839) or a carbamate group at the N-terminus. Also, the N-terminus may be modified by the addition of pyroglutamic acid, or 6-aminohexanoic acid.

The N-terminus of the peptide agonist may be modified by the addition of a group of the formula —SO₂R⁵, to form a sulfonamide group at the N-terminus.

The N-terminus of the peptide agonist may also be modified by reacting with succinic anhydride to form a succinimide group at the N-terminus. The succinimide group incorporates the nitrogen at the N-terminus of the peptide.

The N-terminus may alternatively be modified by the addition of methionine sulfoxide, biotinyl-6-aminohexanoic acid, or —C(═NH)—NH₂. The addition of —C(═NH)—NH₂ is a guanidation modification, where the terminal NH₂ of the N-terminal amino acid becomes —NH—C(═NH)—NH₂.

Most of the sequences of the present invention, including the N-terminal modifications and the C-terminal extensions, contain the standard single letter or three letter codes for the twenty naturally occurring amino acids. The other codes used are defined as follows:
C6=hexanoyl
d=the D isoform (non-naturally occurring) of the respective amino acid, e.g., dA=D-alanine, dS=D-serine, dK=D-lysine
hR=homoarginine
Aib=amino isobutyric acid
OMe=methoxy
Nle=Nor-leucine
NMe=N-methyl attached to the alpha amino group of an amino acid, e.g., NMeA=N-methyl alanine, NMeV=N-methyl valine
Orn=ornithine
K(CO(CH₂)₂SH)=ε-(3′-mercaptopropionyl)-lysine
K(W)=ε-(L-tryptophyl)-lysine
Abu=α-amino-n-butyric acid or 2-aminobutanoic acid
Cit=citrulline
K(Ac)=ε-acetyl lysine
Pyr=pyroglutamic acid
Aha=6-aminohexanoic acid

VIP naturally occurs as a single sequence having 28 amino acids. However, PACAP exists as either a 38 amino acid peptide (PACAP-38) or as a 27 amino acid peptide (PACAP-27) with an amidated carboxyl (Miyata, et al., Biochem Biophys Res Commun, 170:643-648 (1990)). The sequences for VIP, PACAP-27, and PACAP-38 are as follows:

| | | |
|---|---|---|
| Peptide | Seq.ID # | Sequence |

| | | |
|---|---|---|
| VIP | SEQ ID NO: 14 | HSDAVFTDNYTRLRKQMAVKKYLNSILN |
| PACAP- | SEQ ID NO: 15 | HSDGIFTDSYSRYRKQMAVKKYLAAVL-NH₂ |
| 27 | | |
| PACAP- | SEQ ID NO: 16 | HSDGIFTDSYSRYRKQMAVKKYLAAVLGKRYQRVKNK- |
| 38 | | NH₂ |

The term “naturally occurring amino acid” as used herein means the twenty amino acids coded for by the human genetic code (i.e. the twenty standard amino acids). These twenty amino acids are: Alanine, Arginine, Asparagine, Aspartic Acid, Cysteine, Glutamine, Glutamic Acid, Glycine, Histidine, Isoleucine, Leucine, Lysine, Methionine, Phenylalanine, Proline, Serine, Threonine, Tryptophan, Tyrosine and Valine.

Examples of “non-naturally occurring amino acids” include both synthetic amino acids and those modified by the body. These include D-amino acids, arginine-like amino acids (e.g., homoarginine), other amino acids having an extra methylene in the side chain (“homo” amino acids), and modified amino acids (e.g norleucine, lysine (isopropyl)—wherein the side chain amine of lysine is modified by an isopropyl group). Also included are amino acids such as ornithine, amino isobutyric acid and 2-aminobutanoic acid.

“Selective” as used herein refers to a VPAC2 receptor peptide agonist with increased selectivity for the VPAC2 receptor compared to other known receptors. The degree of selectivity is determined by a ratio of VPAC2 receptor binding affinity to VPAC1 receptor binding affinity and by a ratio of VPAC2 receptor binding affinity to PAC1 receptor binding affinity. Binding affinity is determined as described below in Example 4.

“Insulinotropic activity” refers to the ability to stimulate insulin secretion in response to elevated glucose levels, thereby causing glucose uptake by cells and decreased plasma glucose levels. Insulinotropic activity can be assessed by methods known in the art, including using experiments that measure VPAC2 receptor binding activity or receptor activation (e.g. insulin secretion by insulinoma cell lines or islets, intravenous glucose tolerance test (IVGTT), intraperitoneal glucose tolerance test (IPGTT), and oral glucose tolerance test (OGTT)). Insulinotropic activity is routinely measured in humans by measuring insulin levels or C-peptide levels. Selective VPAC2 receptor peptide agonists of the present invention have insulinotropic activity.

“In vitro potency” as used herein is the measure of the ability of a peptide to activate the VPAC2 receptor in a cell-based assay. In vitro potency is expressed as the “EC₅₀” which is the effective concentration of compound that results in a 50% of maximum increase in activity in a single dose-response experiment. For the purposes of the present invention, in vitro potency is determined using two different assays: DiscoveRx and Alpha Screen. See Examples 3 and 5 for further details of these assays. Whilst these assays are performed in different ways, the results demonstrate a general correlation between the two assays.

“Percent (%) sequence identity” as used herein is used to denote sequences which when aligned have similar (identical or conservatively replaced) amino acids in like positions or regions, where identical or conservatively replaced amino acids are those which do not alter the activity or function of the protein as compared to the starting protein. For example, two amino acid sequences with at least 85% identity to each other have at least 85% similar (identical or conservatively replaced residues) in a like position when aligned optimally allowing for up to 3 gaps, with the proviso that in respect of the gaps a total of not more than 15 amino acid residues is affected.

The reference peptide used for the percentage sequence identity calculations herein is:

| | |
|---|---|
| P487 | C6- |
| SEQ ID | HSDAVFTEQY(OMe)TOrnLRAibQLAAAibOrnYLQSIOrnOr |
| NO: 55 | nGGPSSGAPPPS-NH₂ |

Percent sequence identity may be calculated by determining the number of residues that differ between a peptide encompassed by the present invention and a reference peptide such as P487 (SEQ ID NO: 55), taking that number and dividing it by the number of amino acids in the reference peptide (e.g. 39 amino acids for P487), multiplying the result by 100, and subtracting that resulting number from 100. For example, a sequence having 39 amino acids with four amino acids that are different from P487 would have a percent (%) sequence identity of 90% (e.g. 100−((4/39)×100)). For a sequence that is longer than 39 amino acids, the number of residues that differ from the P487 sequence will include the additional amino acids over 39 for purposes of the aforementioned calculation. For example, a sequence having 40 amino acids, with four amino acids different from the 39 amino acids in the P487 sequence and with one additional amino acid at the carboxy terminus which is not present in the P487 sequence, would have a total of five amino acids that differ from P487. Thus, this sequence would have a percent (%) sequence identity of 87% (e.g. 100−((5/39)×100)). The degree of sequence identity may be determined using methods well known in the art (see, for example, Wilbur, W. J. and Lipman, D. J., Proc. Natl. Acad. Sci. USA 80:726-730 (1983) and Myers E. and Miller W., Comput. Appl. Biosci. 4:11-17 (1988)). One program which may be used in determining the degree of similarity is the MegAlign Lipman-Pearson one pair method (using default parameters) which can be obtained from DNAstar Inc, 1128, Selfpark Street, Madison, Wis., 53715, USA as part of the Lasergene system. Another program, which may be used, is Clustal W. This is a multiple sequence alignment package developed by Thompson et al (Nucleic Acids Research, 22(22):4673-4680(1994)) for DNA or protein sequences. This tool is useful for performing cross-species comparisons of related sequences and viewing sequence conservation. Clustal W is a general purpose multiple sequence alignment program for DNA or proteins. It produces biologically meaningful multiple sequence alignments of divergent sequences. It calculates the best match for the selected sequences, and lines them up so that the identities, similarities and differences can be seen. Evolutionary relationships can be seen via viewing Cladograms or Phylograms.

The sequence for a selective VPAC2 receptor peptide agonist of the present invention is selective for the VPAC2 receptor and preferably has a sequence identity in the range of 60% to 70%, 60% to 65%, 65% to 70%, 70% to 80%, 70% to 75%, 75% to 80%, 80% to 90%, 80% to 85%, 85% to 90%, 90% to 97%, 90% to 95%, or 95% to 97%, with P487 (SEQ ID NO: 55). Preferably, the sequence has a sequence identity of greater than 82% with P487 (SEQ ID NO: 55). More preferably, the sequence has greater than 90% sequence identity with P487 (SEQ ID NO: 55). Even more preferably, the sequence has greater than 92% sequence identity with P487 (SEQ ID NO: 55). Yet more preferably, the sequence has greater than 95% sequence identity or 97% sequence identity with P487 (SEQ ID NO: 55).

The term “C₁-C₁₆ alkyl” as used herein means a monovalent saturated straight, branched or cyclic chain hydrocarbon radical having from 1 to 16 carbon atoms or when cyclic, having from 3 to 16 carbon atoms. Thus the term “C₁-C₁₆ alkyl” includes, for example, methyl, ethyl, n-propyl, isopropyl, n-butyl, isobutyl, sec-butyl, tert-butyl, n-heptyl, n-octyl, cyclopropyl, cyclobutyl, cyclopentyl and cyclohexyl. The C₁-C₁₆ alkyl group may be optionally substituted with one or more substituents including, for example, aryl, C₁-C₆ alkoxy, —OH, halogen, —CF₃ and —SH.

The term “C₁-C₆ alkyl” as used herein means a monovalent saturated straight, branched or cyclic chain hydrocarbon radical having from 1 to 6 carbon atoms or when cyclic, having from 3 to 6 carbon atoms. Thus the term “C₁-C₆ alkyl” includes, for example, methyl, ethyl, n-propyl, isopropyl, n-butyl, isobutyl, sec-butyl, tert-butyl, cyclopropyl, cyclobutyl, cyclopentyl and cyclohexyl. The C₁-C₆ alkyl group may be optionally substituted with one or more substituents.

The term “C₂-C₆ alkenyl” as used herein means a monovalent straight, branched or cyclic chain hydrocarbon radical having at least one double bond and having from 2 to 6 carbon atoms or when cyclic, having from 3 to 6 carbon atoms. Thus the term “C₂-C₆ alkenyl” includes vinyl, prop-2-enyl, but-3-enyl, pent-4-enyl and isopropenyl. The C₂-C₆ alkenyl group may be optionally substituted with one or more substituents.

The term “C₂-C₆ alkynyl” as used herein means a monovalent straight or branched chain hydrocarbon radical having at least one triple bond and having from 2 to 6 carbon atoms. Thus the term “C₂-C₆ alkynyl” includes prop-2-ynyl, but-3-ynyl and pent-4-ynyl. The C₂-C₆ alkynyl may be optionally substituted with one or more substituents.

The term “C₁-C₆ alkoxy” as used herein means a monovalent unsubstituted saturated straight-chain or branched-chain hydrocarbon radical having from 1 to 6 carbon atoms linked to the point of substitution by a divalent O radical. Thus the term “C₁-C₆ alkoxy” includes, for example, methoxy, ethoxy, n-propoxy, isopropoxy, n-butoxy, isobutoxy, sec-butoxy and tert-butoxy. The C₁-C₆ alkoxy group may be optionally substituted with one or more substituents.

The term “halo” or “halogen” means fluorine, chlorine, bromine or iodine.

The term “aryl” when used alone or as part of a group is a 5 to 10 membered aromatic or heteroaromatic group including a phenyl group, a 5 or 6-membered monocyclic heteroaromatic group, each member of which may be optionally substituted with 1, 2, 3, 4 or 5 substituents (depending upon the number of available substitution positions), a naphthyl group or an 8-, 9- or 10-membered bicyclic heteroaromatic group, each member of which may be optionally substituted with 1, 2, 3, 4, 5 or 6 substituents (depending on the number of available substitution positions). Within this definition of aryl, suitable substitutions include C₁-C₆ alkyl, C₂-C₆ alkenyl, C₂-C₆ alkynyl, —NH₂, —OH, halogen, —SH and CF₃.

The term “aryl C₁-C₄ alkyl” as used herein means a C₁-C₄ alkyl group substituted with an aryl. Thus the term “aryl C₁-C₄ alkyl” includes benzyl, 1-phenylethyl (α-methylbenzyl), 2-phenylethyl, 1-naphthalenemethyl or 2-naphthalenemethyl.

The term “naphthyl” includes 1-naphthyl, and 2-naphthyl. 1-naphthyl is preferred.

The term “benzyl” as used herein means a monovalent unsubstituted phenyl radical linked to the point of substitution by a —CH₂— group.

The term “5- or 6-membered monocyclic heteroaromatic group” as used herein means a monocyclic aromatic group with a total of 5 or 6 atoms in the ring wherein from 1 to 4 of those atoms are each independently selected from N, O and S. Preferred groups have 1 or 2 atoms in the ring which are each independently selected from N, O and S. Examples of 5-membered monocyclic heteroaromatic groups include pyrrolyl (also called azolyl), furanyl, thienyl, pyrazolyl (also called 1H-pyrazolyl and 1,2-diazolyl), imidazolyl, oxazolyl (also called 1,3-oxazolyl), isoxazolyl (also called 1,2-oxazolyl), thiazolyl (also called 1,3-thiazolyl), isothiazolyl (also called 1,2-thiazolyl), triazolyl, oxadiazolyl, thiadiazolyl, tetrazolyl, oxatriazolyl and thiatriazolyl. Examples of 6-membered monocyclic heteroaromatic groups include pyridinyl, pyrimidyl, pyrazinyl, pyridazinyl and triazinyl.

The term “8-, 9- or 10-membered bicyclic heteroaromatic group” as used herein means a fused bicyclic aromatic group with a total of 8, 9 or 10 atoms in the ring system wherein from 1 to 4 of those atoms are each independently selected from N, O and S. Preferred groups have from 1 to 3 atoms in the ring system which are each independently selected from N, O and S. Suitable 8-membered bicyclic heteroaromatic groups include imidazo[2,1-b][1,3]thiazolyl, thieno[3,2-b]thienyl, thieno[2,3-d][1,3]thiazolyl and thieno[2,3-d]imidazolyl. Suitable 9-membered bicyclic heteroaromatic groups include indolyl, isoindolyl, benzofuranyl (also called benzo[b]furanyl), isobenzofuranyl (also called benzo[c]furanyl), benzothienyl (also called benzo[b]thienyl), isobenzothienyl (also called benzo[c]thienyl), indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, thienopyridinyl, purinyl and imidazo[1,2-a]pyridine. Suitable 10-membered bicyclic heteroaromatic groups include quinolinyl, isoquinolinyl, cinnolinyl, quinazolinyl, quinoxalinyl, 1,5-naphthyridyl, 1,6-naphthyridyl, 1,7-naphthyridyl and 1,8-naphthyridyl.

According to a preferred embodiment of the present invention, there is provided a VPAC2 receptor peptide agonist comprising a peptide sequence selected from SEQ ID NO: 17 to 45, 92 to 110 and 145 to 199, and a C-terminal extension selected from: GGPSSGAPPPS (SEQ ID NO: 5), GGPSSGAPPPS-NH₂ (SEQ ID NO: 6), GGPSSGAPPPC (SEQ ID NO: 7), GGPSSGAPPPC-NH₂ (SEQ ID NO: 8), GRPSSGAPPPS (SEQ ID NO: 9), GRPSSGAPPPS-NH₂ (SEQ ID NO: 10), GGPSSGAPPPCC (SEQ ID NO: 11) and GGPSSGAPPPCC-NH₂ (SEQ ID NO: 12). In this embodiment, it is especially preferred that the C-terminal extension is selected from: GGPSSGAPPPS (SEQ ID NO: 5), GGPSSGAPPPS-NH₂ (SEQ ID NO: 6), GGPSSGAPPPC (SEQ ID NO: 7), GGPSSGAPPPC-NH₂ (SEQ ID NO: 8), GGPSSGAPPPCC (SEQ ID NO: 1) and GGPSSGAPPPCC-NH₂ (SEQ ID NO: 12).

According to a more preferred embodiment of the present invention, there is provided a VPAC2 receptor peptide agonist comprising a peptide sequence selected from SEQ ID NO: 17 to 45, 92 to 110 and 145 to 199 and a C-terminal extension selected from: GGPSSGAPPPS (SEQ ID NO: 5), GGPSSGAPPPS-NH₂ (SEQ ID NO: 6), GGPSSGAPPPC (SEQ ID NO: 7), GGPSSGAPPPC-NH₂ (SEQ ID NO:8), GRPSSGAPPPS (SEQ ID NO: 9), GRPSSGAPPPS-NH₂ (SEQ ID NO: 10), GGPSSGAPPPCC (SEQ ID NO: 11) and GGPSSGAPPPCC-NH₂ (SEQ ID NO: 12) and wherein the VPAC2 receptor peptide agonist further comprises a N-terminal modification, which modification is the addition of acetyl, propionyl, butyryl, pentanoyl, hexanoyl, methionine, methionine sulfoxide, 3-phenylpropionyl, phenylacetyl, benzoyl, norleucine, D-histidine, isoleucine, 3-mercaptopropionyl, biotinyl-6-aminohexanoic acid (6-aminocaproic acid), and —C(═NH)—NH₂. In this embodiment, it is more preferred that the N-terminal modification is the addition of acetyl or hexanoyl.

According to a preferred embodiment of the present invention, there is provided a VPAC2 receptor peptide agonist comprising an amino acid sequence of Formula 4 (SEQ ID NO: 4) and a C-terminal extension selected from GGPSSGAPPPS (SEQ ID NO: 5), GGPSSGAPPPS-NH₂ (SEQ ID NO: 6), GGPSSGAPPPC (SEQ ID NO: 7), GGPSSGAPPPC-NH₂ (SEQ ID NO: 8), GRPSSGAPPPS (SEQ ID NO: 9), GRPSSGAPPPS-N₂ (SEQ ID NO: 10), GGPSSGAPPPCC (SEQ ID NO: 11) and GGPSSGAPPPCC-NH₂ (SEQ ID NO: 12), and wherein the VPAC2 receptor peptide agonist further comprises a N-terminal modification, which modification is the addition of acetyl, propionyl, butyryl, pentanoyl, hexanoyl, methionine, methionine sulfoxide, 3-phenylpropionyl, phenylacetyl, benzoyl, norleucine, D-histidine, isoleucine, 3-mercaptopropionyl, biotinyl-6-aminohexanoic acid (6-aminocaproic acid), and —C(═NH)—NH₂. In this embodiment, it is more preferred that the N-terminal modification is the addition of acetyl or hexanoyl.

According to a more preferred embodiment of the present invention, there is provided a VPAC2 receptor peptide agonist comprising an amino acid sequence of Formula 4 (SEQ ID NO: 4), wherein Xaa₁₅ is Aib, Xaa₂₀ is Aib and Xaa₁₂, Xaa₂₁, Xaa₂₇ and Xaa₂₈ are all Orn, and a C-terminal extension selected from GGPSSGAPPPS (SEQ ID NO: 5), GGPSSGAPPPS-NH₂ (SEQ ID NO: 6), GGPSSGAPPPC (SEQ ID NO: 7), GGPSSGAPPPC-NH₂ (SEQ ID NO: 8), GRPSSGAPPPS (SEQ ID NO: 9), GRPSSGAPPPS-NH₂ (SEQ ID NO: 10), GGPSSGAPPPCC (SEQ ID NO: 11) and GGPSSGAPPPCC-NH₂ (SEQ ID NO: 12), and wherein the VPAC2 receptor peptide agonist further comprises a N-terminal modification, which modification is the addition of acetyl, propionyl, butyryl, pentanoyl, hexanoyl, methionine, methionine sulfoxide, 3-phenylpropionyl, phenylacetyl, benzoyl, norleucine, D-histidine, isoleucine, 3-mercaptopropionyl, biotinyl-6-aminohexanoic acid (6-aminocaproic acid), and —C(═NH)—NH₂. In this embodiment, it is more preferred that Xaa₁₅ is Aib, Xaa₂₀ is Aib, Xaa₁₂, Xaa₂₁, Xaa₂₇ and Xaa₂₈ are all Orn, Xaa₉ is Glu, Xaa₉ is Gln and Xaa₁₀ is Tyr(OMe). It is especially preferred that Xaa₁₅ is Aib, Xaa₂₀ is Aib, Xaa₁₂, Xaa₂₁, Xaa₂₇ and Xaa₂₈ are all Orn, Xaa₈ is Glu, Xaa₉ is Gln, Xaa₁₀ is Tyr(OMe), and Xaa₂₃ and/or Xaa₂₅ is Aib.

The present invention encompasses the discovery that specific amino acids added to the C-terminus of a peptide sequence for a VPAC2 receptor peptide agonist may protect the peptide as well as may enhance activity, selectivity, and/or potency. For example, these C-terminal extensions may stabilize the helical structure of the peptide and stabilize sites located near to the C-terminus, which are prone to enzymatic cleavage. Furthermore, many of the C-terminally extended peptides disclosed herein may be more selective for the VPAC2 receptor and may be more potent than VIP, PACAP, and other known VPAC2 receptor peptide agonists. An example of a preferred C-terminal extension is the extension peptide of exendin-4 as the C-capping sequence. Exendin-4 is found in the salivary excretions from the Gila Monster, Heloderma Suspectum, (Eng et al., J. Biol. Chem., 267(11):7402-7405 (1992)). Other examples of C-terminal extensions are the C-terminal sequences of helodermin and helospectin. Helodermin and helospectin are also found in the salivary excretions of the Gila Monster.

It has furthermore been discovered that modification of the N-terminus of the VPAC2 receptor peptide agonist may enhance potency and/or provide stability against DPP-IV cleavage.

VIP and some known VPAC2 receptor peptide agonists are susceptible to cleavage by various enzymes and, thus, have a short in vivo half-life. Various enzymatic cleavage sites in the VPAC2 receptor peptide agonists are discussed below. The cleavage sites are discussed relative to the amino acid positions in VIP (SEQ ID NO: 14), and are applicable to the sequences noted herein.

Cleavage of the peptide agonist by the enzyme dipeptidyl-peptidase-IV (DPP-IV) occurs between position 2 (serine in VIP) and position 3 (aspartic acid in VIP). The compounds of the present invention may be rendered more stable to DPP-IV cleavage in this region by the addition of a N-terminal modification. Examples of N-terminal modifications that may improve stability against DPP-IV cleavage include the addition of acetyl, propionyl, butyryl, pentanoyl, hexanoyl, methionine, methionine sulfoxide, 3-phenylpropionyl, phenylacetyl, benzoyl, norleucine, D-histidine, isoleucine, 3-mercaptopropionyl, biotinyl-6-aminohexanoic acid, or —C(═NH₂)—NH₂. Preferably, the N-terminal modification is the addition of acetyl or hexanoyl.

There are chymotrypsin cleavage sites in wild-type VIP between the amino acids 10 and 11 (tyrosine and threonine) and those at 22 and 23 (tyrosine and leucine). Making substitutions at position 10 and/or 11 and position 22 and/or 23 may increase the stability of the peptide at these sites. For example, substitution of tyrosine at position 10 and/or position 22 with Tyr(OMe) may increase stability.

There is a trypsin cleavage site between the amino acids at positions 12 and 13 of wild-type VIP. Certain amino acids render the peptide less susceptible to cleavage at this site, for example, ornithine and homoarginine at position 12 and amino isobutyric acid at position 13.

In wild-type VIP, and in numerous VPAC2 receptor peptide agonists known in the art, there are cleavage sites between the basic amino acids at positions 14 and 15 and between those at positions 20 and 21. The selective VPAC2 receptor peptide agonists of the present invention may have improved proteolytic stability in-vivo due to substitutions at these sites. The preferred substitutions at these sites are those which render the peptide less susceptible to cleavage by trypsin-like enzymes, including trypsin. For example, leucine at position 14, amino isobutyric acid at position 15, amino isobutyric acid and glutamine at position 20, and ornithine at position 21 are all preferred substitutions which may lead to improved stability.

There is also a cleavage site between the amino acids at positions 25 and 26 of wild type VIP. The region of the VPAC2 receptor peptide agonist encompassing the amino acids at positions 27, 28, and 29 is also susceptible to enzyme cleavage. The addition of a C-terminal extension may render the peptide agonist more stable against neuroendopeptidase (NEP). This region may also be attacked by trypsin-like enzymes. If that occurs, the peptide agonist may lose its C-terminal extension with the additional carboxypeptidase activity leading to an inactive form of the peptide. Preferred substitutions which may increase resistance to cleavage in this region include ornithine at position 27, ornithine, amino isobutyric acid, or glutamine at position 28 and ornithine, or lysine at position 29.

In addition to selective VPAC2 receptor peptide agonists with resistance to cleavage by various peptidases, the selective VPAC2 peptide receptor agonists of the present invention may also encompass peptides with enhanced selectivity for the VPAC2 receptor, increased potency, and/or increased stability compared with some peptides known in the art. The potency and selectivity of various VPAC2 receptor peptide agonists of the present invention is reported in Examples 3, 4 and 5.

Table 1 in Example 3 provides a list of selective VPAC2 receptor peptide agonists and their corresponding in vitro potency results. Preferably, the selective VPAC2 receptor peptide agonists of the present invention have an EC₅₀ value less than 10 nM. More preferably, the selective VPAC2 receptor peptide agonists of the present invention have an EC₅₀ value less than 2 nM. Evan more preferably, the EC₅₀ value is less than 1 nM. Still more preferably, the EC₅₀ value is less than 0.5 nM.

Table 2 in Example 4 provides a list of VPAC2 receptor peptide agonists and their corresponding binding affinity results for human VPAC2, VPAC1, and PAC1. See Example 4 for further details of these assays. The degree of selectivity is determined by a ratio of VPAC2 receptor binding affinity to VPAC1 receptor binding affinity and by a ratio of VPAC2 receptor binding affinity to PAC1 receptor binding affinity. Preferably, the agonists of the present invention have a selectivity ratio where the affinity for the VPAC2 receptor is at least 50 times greater than for the VPAC1 and/or for PAC1 receptors. More preferably, this affinity is at least 100 times greater for VPAC2 than for VPAC1 and/or for PAC1. Even more preferably, the affinity is at least 200 times greater for VPAC2 than for VPAC1 and/or for PAC1. Still more preferably, the affinity is at least 500 times greater for VPAC2 than for VPAC1 and/or for PAC1. Yet more preferably, the ratio is at least 1000 times greater for VPAC2 than for VPAC1 and/or for PAC1.

As used herein, “selective VPAC2 receptor peptide agonists” also include pharmaceutically acceptable salts of the agonists described herein. A selective VPAC2 receptor peptide agonist of this invention can possess a sufficiently acidic, a sufficiently basic, or both functional groups, and accordingly react with any of a number of inorganic bases, and inorganic and organic acids, to form a salt. Acids commonly employed to form acid addition salts are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, trifluoroacetic acid, and the like. Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, and the like.

The selective VPAC2 receptor peptide agonists of the present invention are preferably formulated as pharmaceutical compositions. Standard pharmaceutical formulation techniques may be employed such as those described in Remington's Pharmaceutical Sciences, Mack Publishing Company, Easton, Pa. The selective VPAC2 receptor peptide agonists of the present invention may be formulated for administration through the buccal, topical, oral, transdermal, nasal, or pulmonary route, or for parenteral administration.

Parenteral administration can include, for example, systemic administration, such as by intramuscular, intravenous, subcutaneous, intradermal, or intraperitoneal injection. The selective VPAC2 receptor peptide agonists can be administered to the subject in conjunction with an acceptable pharmaceutical carrier, diluent, or excipient as part of a pharmaceutical composition for treating NIDDM, or the disorders discussed below. The pharmaceutical composition can be a solution or, if administered parenterally, a suspension of the VPAC2 receptor peptide agonist or a suspension of the VPAC2 receptor peptide agonist complexed with a divalent metal cation such as zinc. Suitable pharmaceutical carriers may contain inert ingredients which do not interact with the peptide or peptide derivative. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Some examples of suitable excipients include lactose, dextrose, sucrose, trehalose, sorbitol, and mannitol.

The VPAC2 receptor peptide agonists of the invention may be formulated for administration such that blood plasma levels are maintained in the efficacious range for extended time periods. The main barrier to effective oral peptide drug delivery is poor bioavailability due to degradation of peptides by acids and enzymes, poor absorption through epithelial membranes, and transition of peptides to an insoluble form after exposure to the acidic pH environment in the digestive tract. Oral delivery systems for peptides such as those encompassed by the present invention are known in the art. For example, VPAC2 receptor peptide agonists can be encapsulated into microspheres composed of a commercially available, biocompatible, biodegradable polymer, poly(lactide-co-glycolide)-COOH and olive oil as a filler (see Joseph, et al. Diabetologia 43:1319-1328 (2000)). Other types of microsphere technology are also available commercially such as Medisorb® and Prolease® biodegradable polymers from Alkermes. Medisorb® polymers can be produced with any of the lactide isomers. Lactide:glycolide ratios can be varied between 0:100 and 100:0 allowing for a broad range of polymer properties. This allows for the design of delivery systems and implantable devices with resorption times ranging from weeks to months. Emisphere has published numerous articles discussing oral delivery technology for peptides and proteins. For example, see WO 95/28838 by Leone-bay et al. which discloses specific carriers comprised of modified amino acids to facilitate absorption.

The selective VPAC2 receptor peptide agonists described herein can be used to treat subjects with a wide variety of diseases and conditions. Agonists encompassed by the present invention exert their biological effects by acting at a receptor referred to as the VPAC2 receptor. Subjects with diseases and/or conditions that respond favourably to VPAC2 receptor stimulation or to the administration of VPAC2 receptor peptide agonists can therefore be treated with the VPAC2 agonists of the present invention. These subjects are said to “be in need of treatment with VPAC2 agonists” or “in need of VPAC2 receptor stimulation”.

The selective VPAC2 receptor peptide agonists of the present invention may be employed to treat diabetes, including both type 1 and type 2 diabetes (non-insulin dependent diabetes mellitus or NIDDM). The agonists may also be used to treat subjects requiring prophylactic treatment with a VPAC2 receptor agonist, e.g., subjects at risk for developing NIDDM. Such treatment may delay the onset of diabetes and diabetic complications. Additional subjects which may be treated with the agonists of the present invention include those with impaired glucose tolerance (IGT) (Expert Committee on Classification of Diabetes Mellitus, Diabetes Care 22 (Supp. 1):S5, 1999) or impaired fasting glucose (IFG) (Charles, et al., Diabetes 40:796, 1991), subjects whose body weight is about 25% above normal body weight for the subject's height and body build, subjects having one or more parents with NIDDM, subjects who have had gestational diabetes, and subjects with metabolic disorders such as those resulting from decreased endogenous insulin secretion. The selective VPAC2 receptor peptide agonists may be used to prevent subjects with impaired glucose tolerance from proceeding to develop NIDDM, prevent pancreatic β-cell deterioration, induce β-cell proliferation, improve β-cell function, activate dormant β-cells, differentiate cells into β-cells, stimulate β-cell replication, and inhibit β-cell apoptosis. Other diseases and conditions that may be treated or prevented using the VPAC2 receptor peptide agonists of the invention include: Maturity-Onset Diabetes of the Young (MODY) (Herman, et al., Diabetes 43:40, 1994); Latent Autoimmune Diabetes Adult (LADA) (Zimmet, et al., Diabetes Med. 11:299, 1994); gestational diabetes (Metzger, Diabetes, 40:197, 1991); metabolic syndrome X, dyslipidemia, hyperglycemia, hyperinsulinemia, hypertriglyceridemia, and insulin resistance.

The selective VPAC2 receptor peptide agonists of the invention may also be used to treat secondary causes of diabetes (Expert Committee on Classification of Diabetes Mellitus, Diabetes Care 22 (Supp. 1):S5, 1999). Such secondary causes include glucocorticoid excess, growth hormone excess, pheochromocytoma, and drug-induced diabetes. Drugs that may induce diabetes include, but are not limited to, pyriminil, nicotinic acid, glucocorticoids, phenytoin, thyroid hormone, β-adrenergic agents, α-interferon and drugs used to treat HIV infection.

The selective VPAC2 receptor peptide agonists of the present invention may be effective in the suppression of food intake and the treatment of obesity.

The selective VPAC2 receptor peptide agonists of the present invention may also be effective in the prevention or treatment of such disorders as atherosclerotic disease, hyperlipidemia, hypercholesteremia, low HDL levels, hypertension, primary pulmonary hypertension, cardiovascular disease (including atherosclerosis, coronary heart disease, and coronary artery disease), cerebrovascular disease and peripheral vessel disease; and for the treatment of lupus, polycystic ovary syndrome, carcinogenesis, hyperplasia, male and female reproduction problems, sexual disorders, ulcers, sleep disorders, disorders of lipid and carbohydrate metabolism, circadian dysfunction, growth disorders, disorders of energy homeostasis, immune diseases including autoimmune diseases (e.g., systemic lupus erythematosus), as well as acute and chronic inflammatory diseases, rheumatoid arthritis, and septic shock.

The selective VPAC2 receptor peptide agonists of the present invention may also be useful for treating physiological disorders related to, for example, cell differentiation to produce lipid accumulating cells, regulation of insulin sensitivity and blood glucose levels, which are involved in, for example, abnormal pancreatic β-cell function, insulin secreting tumors and/or autoimmune hypoglycemia due to autoantibodies to insulin, autoantibodies to the insulin receptor, or autoantibodies that are stimulatory to pancreatic β-cells, macrophage differentiation which leads to the formation of atherosclerotic plaques, inflammatory response, carcinogenesis, hyperplasia, adipocyte gene expression, adipocyte differentiation, reduction in the pancreatic β-cell mass, insulin secretion, tissue sensitivity to insulin, liposarcoma cell growth, polycystic ovarian disease, chronic anovulation, hyperandrogenism, progesterone production, steroidogenesis, redox potential and oxidative stress in cells, nitric oxide synthase (NOS) production, increased gamma glutamyl transpeptidase, catalase, plasma triglycerides, HDL, and LDL cholesterol levels, and the like.

In addition, the selective VPAC2 receptor peptide agonists of the invention may be used for treatment of asthma (Bolin, et al., Biopolymer 37:57-66 (1995); U.S. Pat. No. 5,677,419; showing that polypeptide R3P0 is active in reducing guinea pig tracheal smooth muscle); for hypotension induction (VIP induces hypotension, tachycardia, and facial flushing in asthmatic patients (Morice, et al., Peptides 7:279-280 (1986); Morice, et al., Lancet 2:1225-1227 (1983)); for the treatment of male reproduction problems (Siow, et al., Arch. Androl. 43(1):67-71 (1999)); as an anti-apoptosis/neuroprotective agent (Brenneman, et al., Ann. N.Y. Acad. Sci. 865:207-12 (1998)); for cardioprotection during ischemic events (Kalfin, et al., J. Pharmacol. Exp. Ther. 1268(2):952-8 (1994); Das, et al., Ann. N.Y. Acad. Sci. 865:297-308 (1998)); for manipulation of the circadian clock and its associated disorders (Hamar, et al., Cell 109:497-508 (2002); Shen, et al., Proc. Natl. Acad. Sci. 97:11575-80, (2000)); and as an anti-ulcer agent (Tuncel, et al., Ann. N.Y. Acad. Sci. 865:309-22, (1998)).

An “effective amount” of a selective VPAC2 receptor peptide agonist is the quantity that results in a desired therapeutic and/or prophylactic effect without causing unacceptable side effects when administered to a subject in need of VPAC2 receptor stimulation. A “desired therapeutic effect” includes one or more of the following: 1) an amelioration of the symptom(s) associated with the disease or condition; 2) a delay in the onset of symptoms associated with the disease or condition; 3) increased longevity compared with the absence of the treatment; and 4) greater quality of life compared with the absence of the treatment. For example, an “effective amount” of a VPAC2 agonist for the treatment of NIDDM is the quantity that would result in greater control of blood glucose concentration than in the absence of treatment, thereby resulting in a delay in the onset of diabetic complications such as retinopathy, neuropathy, or kidney disease. An “effective amount” of a selective VPAC2 receptor peptide agonist for the prevention of NIDDM is the quantity that would delay, compared with the absence of treatment, the onset of elevated blood glucose levels that require treatment with anti-hypoglycemic drugs such as sulfonylureas, thiazolidinediones, insulin, and/or bisguanidines.

An “effective amount” of the selective VPAC2 receptor peptide agonist administered to a subject will also depend on the type and severity of the disease and on the characteristics of the subject, such as general health, age, sex, body weight and tolerance to drugs. The dose of selective VPAC2 peptide receptor agonist effective to normalize a patient's blood glucose will depend on a number of factors, among which are included, without limitation, the subject's sex, weight and age, the severity of inability to regulate blood glucose, the route of administration and bioavailability, the pharmacokinetic profile of the peptide, the potency, and the formulation.

A typical dose range for the selective VPAC2 receptor peptide agonists of the present invention will range from about 1 μg per day to about 5000 μg per day. Preferably, the dose ranges from about 1 μg per day to about 2500 μg per day, more preferably from about 1 μg per day to about 1000 μg per day. Even more preferably, the dose ranges from about 5 μg per day to about 100 μg per day. A further preferred dose range is from about 10 μg per day to about 50 μg per day. Most preferably, the dose is about 20 μg per day.

A “subject” is a mammal, preferably a human, but can also be an animal, e.g., companion animals (e.g., dogs, cats, and the like), farm animals (e.g., cows, sheep, pigs, horses, and the like) and laboratory animals (e.g., rats, mice, guinea pigs, and the like).

The selective VPAC2 receptor peptide agonists of the present invention can be prepared by using standard methods of solid-phase peptide synthesis techniques. Peptide synthesizers are commercially available from, for example, Rainin-PTI Symphony Peptide Synthesizer (Tucson, Ariz.). Reagents for solid phase synthesis are commercially available, for example, from Glycopep (Chicago, Ill.). Solid phase peptide synthesizers can be used according to manufacturer's instructions for blocking interfering groups, protecting the amino acid to be reacted, coupling, decoupling, and capping of unreacted amino acids.

Typically, an α-N-protected amino acid and the N-terminal amino acid on the growing peptide chain on a resin is coupled at room temperature in an inert solvent such as dimethylformamide, N-methylpyrrolidone or methylene chloride in the presence of coupling agents such as dicyclohexylcarbodiimide and 1-hydroxybenzotriazole and a base such as diisopropylethylamine. The α-N-protecting group is removed from the resulting peptide resin using a reagent such as trifluoroacetic acid or piperidine, and the coupling reaction repeated with the next desired N-protected amino acid to be added to the peptide chain. Suitable amine protecting groups are well known in the art and are described, for example, in Green and Wuts, “Protecting Groups in Organic Synthesis”, John Wiley and Sons, 1991. Examples include t-butyloxycarbonyl (tBoc) and fluorenylmethoxycarbonyl (Fmoc).

The selective VPAC2 receptor peptide agonists may also be synthesized using standard automated solid-phase synthesis protocols using t-butoxycarbonyl- or fluorenylmethoxycarbonyl-alpha-amino acids with appropriate side-chain protection. After completion of synthesis, modification of the N-terminus may be accomplished by reacting the α-amino group with, for example: (i) active esters (using similar protocols as described above for the introduction of an α-N-protected amino acid); (ii) aldehydes in presence of a reducing agent (reductive amination procedure); and (iii) guanidation reagents. Then, peptides are cleaved from the solid-phase support with simultaneous side-chain deprotection using standard hydrogen fluoride methods or trifluoroacetic acid (TFA). Crude peptides are then further purified using Reversed-Phase Chromatography on VYDAC C18 columns using acetonitrile gradients in 0.1% trifluoroacetic acid (TFA). To remove acetonitrile, peptides are lyophilized from a solution containing 0.1% TFA, acetonitrile and water. Purity can be verified by analytical reversed phase chromatography. Identity of peptides can be verified by mass spectrometry. Peptides can be solubilized in aqueous buffers at neutral pH.

The peptide agonists of the present invention may also be made by recombinant methods known in the art using both eukaryotic and prokaryotic cellular hosts.

Various preferred features and embodiments of the present invention will now be described with reference to the following non-limiting examples.

### EXAMPLE 1

### Preparation of the Selective VPAC2 Receptor Peptide Agonists by Solid Phase t-Boc Chemistry

Approximately 0.5-0.6 grams (0.38-0.45 mmole) Boc Ser(Bzl)-PAM resin is placed in a standard 60 mL reaction vessel. Double couplings are run on an Applied Biosystems ABI430A peptide synthesizer.

The following side-chain protected amino acids (2 mmole cartridges of Boc amino acids) are obtained from Midwest Biotech (Fishers, Ind.) and are used in the synthesis:

Arg-Tosyl (TOS), Asp-δ-cyclohexyl ester (OcHx), Glu-δ-cycohexyl ester (OcHx), His-benzyloxymethyl(BOM), Lys-2-chlorobenzyloxycarbonyl (2Cl-Z), Ser-O-benzyl ether (OBzl), Thr-O-benzyl ether (OBzl), Trp-formyl (CHO), and Tyr-2-bromobenzyloxycarbonyl (2Br-Z).

Trifluoroacetic acid (TFA), di-isopropylethylamine (DIEA), 0.5 M hydroxybenzotriazole (HOBt) in DMF and 0.5 M dicyclohexylcarbodiimide (DCC) in dichloromethane are purchased from PE-Applied Biosystems (Foster City, Calif.). Dimethylfornamide (DMF-Burdick and Jackson) and dichloromethane (DCM-Mallinkrodt) are purchased from Mays Chemical Co. (Indianapolis, Ind.).

Standard double couplings are run using either symmetric anhydride or HOBt esters, both formed using DCC. At the completion of the syntheses, the N-terminal Boc group is removed and the peptidyl resins are treated with 20% piperidine in DMF to deformylate the Trp side chain if Trp is present in the sequence. For N-terminal acylation, four-fold excess of symmetric anhydride of the corresponding acid is added onto the peptide resin. The symmetric anhydride is prepared by diisopropylcarbodiimide (DIC) activation in DCM. The reaction is allowed to proceed for 4 hours and monitored by ninhydrin test. After washing with DCM, the resins are transferred to a TEFLON reaction vessel and are dried in vacuo.

Cleavages are done by attaching the reaction vessels to a HF (hydrofluoric acid) apparatus (Penninsula Laboratories). 1 mL m-cresol per gram/resin is added and 10 mL HF (purchased from AGA, Indianapolis, Ind.) is condensed into the pre-cooled vessel. 1 mL DMS per gram resin is added when methionine is present. The reactions are stirred one hour in an ice bath. The HF is removed in vacuo. The residues are suspended in ethyl ether. The solids are filtered and are washed with ether. Each peptide is extracted into aqueous acetic acid and either is freeze dried or is loaded directly onto a reverse-phase column.

Purifications are run on a 2.2×25 cm VYDAC C18 column in buffer A (0.1% TFA in water). A gradient of 20% to 90% B (0.1% TFA in acetonitrile) is run on an HPLC (Waters) over 120 minutes at 10 mL/minute while monitoring the UV at 280 nm (4.0 A) and collecting one minute fractions. Appropriate fractions are combined, frozen and lyophilized. Dried products are analyzed by HPLC (0.46×15 cm METASIL AQ C18) and MALDI mass spectrometry.

### EXAMPLE 2

### Preparation of the Selective VPAC2 Receptor Peptide Agonists by Solid Phase FMoc Chemistry

Approximately 114 mg (50 mMole) FMOC Ser(tBu) WANG resin (purchased from GlycoPep, Chicago, Ill.) is placed in each reaction vessel. The synthesis is conducted on a Rainin Symphony Peptide Synthesizer. Analogs with a C-terminal amide are prepared using 75 mg (50 μmole) Rink Amide AM resin (Rapp Polymere. Tuebingen, Germany).

The following FMOC amino acids are purchased from GlycoPep (Chicago, Ill.), and NovaBiochem (La Jolla, Calif.): Arg-2,2,4,6,7-pentamethyldihydrobenzofuran-5-sulfonyl (Pbf), Asn-trityl (Trt), Asp-β-t-Butyl ester (tBu), Glu-δ-t-butyl ester (tBu), Gln-trityl (Trt), His-trityl (Trt), Lys-t-butyloxycarbonyl (Boc), Ser-t-butyl ether (OtBu), Thr-t-butyl ether (OtBu), Trp-t-butyloxycarbonyl (Boc), Tyr-t-butyl ether (OtBu).

Solvents dimethylformamide (DMF-Burdick and Jackson), N-methyl pyrrolidone (NMP-Burdick and Jackson), dichloromethane (DCM-Mallinkrodt) are purchased from Mays Chemical Co. (Indianapolis, Ind.).

Hydroxybenzotriazole (HOBt), di-isopropylcarbodiimide (DIC), di-isopropylethylamine (DIEA), and piperidine (Pip) are purchased from Aldrich Chemical Co (Milwaukee, Wis.).

All amino acids are dissolved in 0.3 M in DMF. Three hour DIC/HOBt activated couplings are run after 20 minutes deprotection using 20% Piperidine/DMF. Each resin is washed with DMF after deprotections and couplings. After the last coupling and deprotection, the peptidyl resins are washed with DCM and are dried in vacuo in the reaction vessel. For N-terminal acylation, four-fold excess of symmetric anhydride of the corresponding acid is added onto the peptide resin. The symmetric anhydride is prepared by DIC activation in DCM. The reaction is allowed to proceed for 4 hours and monitored by ninhydrin test. The peptide resin is then washed with DCM and dried in vacuo.

The cleavage reaction is mixed for 2 hours with a cleavage cocktail consisting of 0.2 mL thioanisole, 0.2 mL methanol, 0.4 mL triisopropylsilane, per 10 mL TFA, all purchased from Aldrich Chemical Co., Milwaukee, Wis. If Cys is present in the sequence, 2% of ethanedithiol is added. The TFA filtrates are added to 40 mL ethyl ether. The precipitants are centrifuged 2 minutes at 2000 rpm. The supernatants are decanted. The pellets are resuspended in 40 mL ether, re-centrifuged, re-decanted, dried under nitrogen and then in vacuo.

0.3-0.6 mg of each product is dissolved in 1 mL 0.1% TFA/acetonitrile(ACN), with 20 μL being analyzed on HPLC [0.46×15 cm METASIL AQ C18, 1 mL/min, 45° C., 214 nM (0.2 A), A=0.1% TFA, B=0.1% TFA/50% ACN. Gradient=50% B to 90% B over 30 minutes].

Purifications are run on a 2.2×25 cm VYDAC C18 column in buffer A (0.1% TFA in water). A gradient of 20% to 90% B (0.1% TFA in acetonitrile) is run on an HPLC (Waters) over 120 minutes at 10 mL/minute while monitoring the UV at 280 nm (4.0 A) and collecting 1 minute fractions. Appropriate fractions are combined, frozen and lyophilized. Dried products are analyzed by HPLC (0.46×15 cm METASIL AQ C18) and MALDI mass spectrometry.

P442: Synthesis is carried out using the FMoc protocols described above. P442 is characterised by analytical HPLC: t_{R}=10.8 min, HPLC conditions as described above, and MALDI-TOF: calculated m/z=4273.9, measured m/z=4274.9 [M+H⁺]. After purification using reversed-phase preparative HPLC, pure fractions are combined and lyophilised: 5.4 mg is obtained as a final lyophilised powder.

P520: As described for P442. Analytical HPLC: t_{R}=10.9 min. MALDI-TOF: calculated m/z=4290.0, measured m/z=4290.8 [M+H⁺]. 14.8 mg is obtained as a final lyophilised powder.

P524: As described for P442. Analytical HPLC: t_{R}=11.0 min. MALDI-TOF: calculated m/z=4288.0, measured m/z=4288.8 [M+H⁺]. 18.7 mg is obtained as a final lyophilised powder.

P574: As described for P442. Analytical HPLC: t_{R}=11.7 min. MALDI-TOF: calculated m/z=4330.1, measured m/z=4330.7 [M+H⁺]. 46.2 mg is obtained as a final lyophilised powder.

### EXAMPLE 3

### In-Vitro Potency at Human VPAC2 Receptors

DiscoveRx: A CHO-S cell line stably expressing human VPAC2 receptor in a 96-well microtiter plate is seeded with 50,000 cells/well the day before the assay. The cells are allowed to attach for 24 hours in 200 μL culture medium. On the day of the experiment, the medium is removed. Also, the cells are washed twice. The cells are incubated in assay buffer plus IBMX for 15 minutes at room temperature. Afterwards, the stimuli are added and are dissolved in assay buffer. The stimuli are present for 30 minutes. Then, the assay buffer is gently removed. The cell lysis reagent of the DiscoveRx cAMP kit is added. Thereafter, the standard protocol for developing the cAMP signal as described by the manufacturer is used (DiscoveRx Inc., USA). EC₅₀ values for cAMP generation are calculated from the raw signal or are based on absolute cAMP levels as determined by a standard curve performed on each plate. In the case of the VPAC1 receptor, CHO-PO cells are transiently transfected with human VPAC1 receptor DNA using commercially available transfection reagents (Lipofectamine from Invitrogen). The cells are seeded at a density of 10,000/well in a 96-well plate and are allowed to grow for 3 days in 200 mL culture medium. At day 3, the assay described above for the VPAC2 receptor cell line is performed.

Results for each agonist are the mean of two independent runs. VPAC1 results are only generated using the DiscoveRx assay. The typically tested concentrations of peptide are: 1000, 300, 100, 10, 1, 0.3, 0.1, 0.01, 0.001, 0.0001 and 0 nM.

Alpha screen: Cells are washed in the culture flask once with PBS. Then, the cells are rinsed with enzyme free dissociation buffer. The dissociated cells are removed. The cells are then spun down and washed in stimulation buffer. For each data point, 50,000 cells suspended in stimulation buffer are used. To this buffer, Alpha screen acceptor beads are added along with the stimuli. This mixture is incubated for 60 minutes. Lysis buffer and Alpha screen donor beads are added and are incubated for 60 to 120 minutes. The Alpha screen signal (indicative of intracellular cAMP levels) is read in a suitable instrument (e.g. AlphaQuest from Perkin-Elmer). Steps including Alpha screen donor and acceptor beads are performed in reduced light. The EC₅₀ for cAMP generation is calculated from the raw signal or is based on absolute cAMP levels as determined by a standard curve performed on each plate.

Results for each agonist are, at minimum, from two analyses performed in a single run. For some agonists, the results are the mean of more than one run. The tested peptide concentrations are: 10000, 1000, 100, 10, 3, 1, 0.1, 0.01, 0.003, 0.001, 0.0001 and 0.00001 nM.

The activity (EC₅₀ (nM)) for the human VPAC2 and VPAC1 receptors is reported in Table 1.

TABLE 1

| | Human VPAC2 | Human VPAC2 | Human VPAC1 |
|---|---|---|---|
| | Receptor: | Receptor: Alpha | Receptor: |
| | DiscoveRx¹ | Screen² | DiscoveRx¹ |
| Agonist # | (EC₅₀; nM) | (EC₅₀; nM) | (EC₅₀; nM) |
| PACAP-27 | 0.84 | 2.33 | 0.05 |
| VIP | 0.70 | 1.00 | 0.02 |
| VPAC1-P1 | 179.29 | | |
| P29 | 3.02 | | 18.31 |
| P343 | | 0.29 | |
| P357 | | 0.23 | |
| P362 | | 0.58 | |
| P363 | | 0.27 | |
| P367 | | 6.5 | |
| P368 | | 1.09 | |
| P369 | | 10.46 | |
| P370 | | 0.23 | |
| P371 | | 0.13 | |
| P372 | | 0.65 | |
| P382 | | 0.33 | |
| P383 | | 0.12 | |
| P384 | | 6.7 | |
| P385 | | 23.93 | |
| P386 | | 0.65 | |
| P387 | | 0.47 | |
| P388 | | 2760 | |
| P389 | | 0.16 | |
| P390 | | 0.12 | |
| P391 | | 0.48 | |
| P392 | | 0.21 | |
| P393 | | 0.31 | |
| P394 | | 0.11 | |
| P395 | | 0.15 | |
| P396 | | 0.11 | |
| P397 | | 0.15 | |
| P405 | | 0.14 | |
| P406 | | 0.12 | |
| P407 | | 0.12 | |
| P408 | | 0.12 | |
| P409 | | 0.19 | |
| P418 | | 3.11 | |
| P487 | | 0.24 | |
| P488 | | 0.19 | |

¹Mean of two or more independent runs
²Single result from two analyses performed in a single run. Some values represent the average of more than one such experiment.

### EXAMPLE 4

### Selectivity

Binding assays: Membrane prepared from a stable VPAC2 cell line (see Example 3) or from cells transiently transfected with human VPAC1 or PAC1 are used. A filter binding assay is performed using 125I-labeled VIP for VPAC1 and VPAC2 and 125I-labeled PACAP-27 for PAC1 as the tracers.

For this assay, the solutions and equipment include:

Presoak solution: 0.5% Polyethyleneamine in Aqua dest

Buffer for flushing filter plates: 25 mM HEPES pH 7.4

Blocking buffer: 25 mM HEPES pH 7.4; 0.2% protease free BSA

Assay buffer: 25 mM HEPES pH 7.4; 0.5% protease free BSA

Dilution and assay plate: PS-Microplate, U form

Filtration Plate: Multiscreen FB Opaque Plate; 1.0 μM Type B Glasfiber filter

In order to prepare the filter plates, the presoak solution is aspirated by vacuum filtration. The plates are flushed twice with 200 μL flush buffer. 200 μL blocking buffer is added to the filter plate. The filter plate is then incubated with 200 μL presoak solution for 1 hour at room temperature.

The assay plate is filled with 25 μL assay buffer, 25 μL membranes (2.5 μg) suspended in assay buffer, 25 μL compound (agonist) in assay buffer, and 25 μL tracer (about 40000 cpm) in assay buffer. The filled plate is incubated for 1 hour with shaking.

The transfer from assay plate to filter plate is conducted. The blocking buffer is aspirated by vacuum filtration and washed two times with flush buffer. 90 μL is transferred from the assay plate to the filter plate. The 90 μL transferred from assay plate is aspirated and washed three times with 200 μL flush buffer. The plastic support is removed. It is dried for 1 hour at 60° C. 30 μL Microscint is added. The count is performed.

The selectivity (IC₅₀) for human VPAC2, VPAC1, and PAC1 is reported in Table 2.

TABLE 2

| | Human VPAC2 | Human VPAC1 | Human PAC1 |
|---|---|---|---|
| | Receptor Binding | Receptor Binding | Receptor Binding |
| Agonist # | (IC50; nM) | (IC50; nM) | (IC50; nM) |
| PACAP-27 | 2.62 | 4.47 | 10.20 |
| VIP | 5.06 | 3.28 | >1000 |
| P343 | 0.85 | >3000 | >25000 |
| P357 | 0.32 | >3000 | >25000 |
| P362 | 1.91 | >3000 | >25000 |
| P363 | 0.25 | >3000 | >25000 |
| P367 | >100 | >3000 | >25000 |
| P368 | 28.60 | >3000 | >25000 |
| P369 | 37.26 | >3000 | >25000 |
| P370 | 0.80 | >3000 | >25000 |
| P371 | 0.34 | | >25000 |
| P372 | 4.82 | >3000 | >25000 |
| P382 | 2.17 | >3000 | >25000 |
| P383 | 5.01 | >3000 | >25000 |
| P384 | 22.17 | >3000 | >25000 |
| P385 | 99.23 | | >25000 |
| P386 | 5.92 | >3000 | >25000 |
| P387 | 1.39 | >3000 | >25000 |
| P388 | 100.00 | >3000 | >25000 |
| P389 | 0.82 | | >25000 |
| P390 | 0.26 | | >25000 |
| P391 | 3.67 | | >25000 |
| P392 | 0.45 | | >25000 |
| P393 | 0.74 | | >25000 |
| P394 | 0.41 | | >25000 |
| P395 | 0.24 | | >25000 |
| P396 | 0.26 | >3000 | >25000 |
| P397 | 0.50 | >3000 | >25000 |
| P405 | 0.36 | >3000 | >25000 |
| P406 | 0.27 | >3000 | >25000 |
| P408 | 0.48 | >3000 | >25000 |
| P409 | 1.06 | >3000 | >25000 |
| P418 | 8.96 | >3000 | >25000 |
| P487 | 0.64 | >1000 | |
| P488 | 0.64 | >1000 | |

### EXAMPLE 5

### In Vitro Potency at Rat VPAC1 and VPAC2 Receptors

DiscoveRx: CHO-PO cells are transiently transfected with rat VPAC1 or VPAC2 receptor DNA using commercially available transfection reagents (Lipofectamine from Invitrogen). The cells are seeded at a density of 10,000/well in a 96-well plate and are allowed to grow for 3 days in 200 mL culture medium. At day 3, the assay is performed.

On the day of the experiment, the medium is removed. Also, the cells are washed twice. The cells are incubated in assay buffer plus IBMX for 15 minutes at room temperature. Afterwards, the stimuli are added and are dissolved in assay buffer. The stimuli are present for 30 minutes. Then, the assay buffer is gently removed. The cell lysis reagent of the DiscoveRx cAMP kit is added. Thereafter, the standard protocol for developing the cAMP signal as described by the manufacturer is used (DiscoveRx Inc., USA). EC₅₀ values for cAMP generation are calculated from the raw signal or are based on absolute cAMP levels as determined by a standard curve performed on each plate.

Results for each agonist are the mean of two independent runs. Rat VPAC1 and VPAC2 results are only generated using the DiscoveRx assay. The typically tested concentrations of peptide are: 1000, 300, 100, 10, 1, 0.3, 0.1, 0.01, 0.001, 0.0001 and 0 nM.

The activity (EC₅₀ (nM)) for the rat VPAC2 and VPAC1 receptors is reported in Table 3.

TABLE 3

| | Rat VPAC2 | Rat VPAC1 |
|---|---|---|
| | Receptor: | Receptor: |
| | DiscoveRx | DiscoveRx |
| Agonist # | (EC₅₀; nM) | (EC₅₀; nM) |
| PACAP-27 | | 0.07 |
| VIP | 0.61 | 0.01 |
| P343 | 0.15 | 2.62 |
| P357 | 0.07 | 0.69 |
| P362 | 1.22 | 35.93 |
| P363 | 0.09 | 1.44 |
| P367 | 6.14 | 75.31 |
| P368 | 1.33 | 20.90 |
| P369 | 8.09 | 129.84 |
| P370 | 0.05 | 0.05 |
| P372 | 0.18 | 1.67 |
| P382 | 0.37 | 8.21 |
| P383 | 0.18 | 1.68 |
| P384 | 8.05 | >600 |
| P385 | 28.77 | 96.78 |
| P386 | 0.28 | 4.34 |
| P387 | 0.40 | 20.35 |
| P388 | 709.30 | >2000 |
| P389 | 0.21 | 4.40 |
| P390 | 0.09 | 1.34 |
| P391 | 0.50 | 9.09 |
| P392 | 0.08 | 0.71 |
| P393 | 0.17 | 16.03 |
| P394 | 0.03 | 0.48 |
| P395 | 0.03 | 0.35 |
| P396 | 0.05 | 1.05 |
| P405 | 0.04 | 0.54 |
| P406 | 0.04 | 0.27 |
| P407 | 0.03 | 0.20 |
| P408 | 0.04 | 0.35 |
| P409 | 0.13 | 7.65 |
| P418 | 0.57 | 7.76 |
| P487 | 0.03 | 1.42 |
| P488 | 0.09 | 1.66 |

### EXAMPLE 6

### Serum Stability Studies

In order to determine the stability of VPAC2 receptor peptide agonists in rat serum, CHO-VPAC2 cells clone #6 (96 well plates/50,000 cells/well and 1 day culture), PBS 1× (Gibco), the peptides for the analysis in a 100 μM stock solution, rat serum from a sacrificed normal Wistar rat, aprotinin, and a DiscoveRx assay kit are obtained. The rat serum is stored at 4° C. until use and is used within two weeks.

On Day 0, two 100 μL aliquots of 10 μM peptide in rat serum are prepared by adding 10 μL peptide stock to 90 μL rat serum for each aliquot. 250 kIU aprotinin/mL is added to one of these aliquots. The aliquot is stored with aprotinin at 4° C. The aliquot is stored without aprotinin at 37° C. The aliquots are incubated overnight.

On Day 1, after overnight incubation of the aliquots prepared on day 0, an incubation buffer containing PBS+1.3 mM CaCl₂, 1.2 mM MgCl₂, 2 mM glucose, and 0.25 mM IBMX is prepared. A plate with 11 serial 5× dilutions of peptide for the 4° C. and 37° C. aliquot is prepared for each peptide studied. 2000 nM is used as the maximal concentration if the peptide has an EC₅₀ above 1 nM and 1000 nM as maximal concentration if the peptide has an EC₅₀ below 1 nM from the primary screen (see Example 3). The plate(s) are washed with cells twice in incubation buffer. The plates are allowed to hold 50 μL incubation media per well for 15 minutes. 50 μL solution per well is transferred to the cells from the plate prepared with 11 serial 5× dilutions of peptide for the 4° C. and 37° C. aliquot for each peptide studied, using the maximal concentrations that are indicated by the primary screen, in duplicate. This step dilutes the peptide concentration by a factor of two. The cells are incubated at room temperature for 30 minutes. The supernatant is removed. 40 μL/well of the DiscoveRx antibody/extraction buffer is added. The cells are incubated on the shaker (300 rpm) for 1 hour. Normal procedure with the DiscoveRx kit is followed. cAMP standards are included in column 12. EC₅₀ values are determined from the cAMP assay data. The remaining amount of active peptide is estimated by the formula EC_{50, 4C}/EC_{50, 37C}.

Estimated peptide stabilities are shown in table 4 below.

TABLE 4

| | Rat Serum Stability |
|---|---|
| | (estimated purity in % |
| Agonist# | after overnight incub.) |
| P343 | 34.46 |
| P357 | 38.83 |
| P358 | 13.43 |
| P362 | 82.77 |
| P363 | 81.66 |
| P367 | 55.82 |
| P368 | 99.82 |
| P369 | 133.76 |
| P370 | 41.82 |
| P372 | 13.93 |
| P382 | 92.34 |
| P383 | 113.66 |
| P384 | 96.45 |
| P386 | 71.47 |
| P387 | 74.99 |
| P396 | 93.07 |
| P397 | 71.07 |
| P405 | 79.54 |
| P406 | 89.83 |
| P407 | 151.54 |
| P408 | 142.44 |
| P418 | 99.96 |
| P487 | 76.98 |
| P488 | 193.17 |

Serum stability in human serum may also be determined using the above described protocol substituting rat serum for human serum (Eg. Sigma #H-4522, Lot #043 K0500).

The estimated amounts of peptide (%) remaining after 24 h incubation at 37° C. in human serum are listed in table 5 below.

TABLE 5

| | Human Serum |
|---|---|
| | Stability |
| | (estimated purity in |
| Agonist# | % after 24 hours) |
| P372 | 61.71 |
| P396 | 97.92 |
| P397 | 114.28 |
| P418 | 84.54 |

### EXAMPLE 7

### Comparison of the Interaction of the VPAC2 Receptor Peptide Agonists of the Present Invention with the Recombinant Rat VPAC1, VPAC2 and PAC1 Receptors Expressed in CHO Cells

The peptide samples are stored frozen and thawed prior to the assay. Reference compounds (e.g. VIP and the tracers) are not stored frozen. All peptide sample and reference compound dilutions are performed in PBS. Peptides solutions are kept in the cold room for four days. Stock solutions are stored at −80° C. New dilution curves are prepared every week.

All studies are performed on crude membranes prepared from three different cell cultures expressing the different recombinant receptors, using methodology that is known in the literature. Duplicate values are obtained for each assay.

The selectivity of the VPAC2 receptor peptide agonists of the present invention are tested on the rat VPAC1 and VPAC2 receptors recombinantly expressed in CHO cells. The compounds of the invention were evaluated in receptor binding and adenylate cyclase activation assays.

Competition binding curves from 10⁻¹¹ to 10⁻⁵ M (two concentrations per log) of unlabelled peptide using ¹²⁵I-VIP (VPAC1-R) and ¹²⁵I-RO 25-1553 (VPAC2-R) as tracers; incubations performed at 25° C. for 30 minutes. In each series of assays, unlabelled VIP and RO 25-1553 are used as standards. Each assay is done in duplicate and performed on two different membrane preparations.

Dose-effect curves of adenylate cyclase activation are generated using the VPAC2 receptor peptide agonists (10⁻¹¹ to 10⁻⁶ M, two concentrations per log) of the present invention.

Adenylate cyclase activity is determined by the procedure of Salomon et al., a highly sensitive adenylate cyclase assay (Analytical Biochemistry 58 (1974)). Membrane proteins (3-15 g) are incubated in a total volume of 60 1 containing 0.5 mM [³²P]-ATP, 10 M GTP, 5 mM MgCl₂, 0.5 mM EGTA, 1 mM cAMP, 1 mM theophylline, 10 mM phospho(enol)pyruvate, 30 g/ml pyruvate kinase and 30 mM Tris-HCl at a final pH of 7.8. The reaction is initiated by membrane addition and is terminated after 15 min incubation at 37° C. by addition of 0.5 ml of 0.5% sodium dodecyl-sulfate solution containing 0.5 mM ATP, 0.5 mM cAMP and 20,000 g [³H]-cAMP. cAMP was separated from ATP by two successive chromatographies on Dowex 50 W×8 and neutral alumina.

### EXAMPLE 8

### In Vivo Assays

Intravenous glucose tolerance test (IVGTT): Normal Wistar rats are fasted overnight and are anesthetized prior to the experiment. A blood sampling catheter is inserted into the rats. The compound is given in the jugular vein. Blood samples are taken from the carotid artery. A blood sample is drawn immediately prior to the injection of glucose along with the compound. After the initial blood sample, glucose mixed with compound is injected intravenously (i.v.). A glucose challenge of 0.5 g/kg body weight is given, injecting a total of 1.5 mL vehicle with glucose and agonist per kg body weight. The peptide concentrations are varied to produce the desired dose in μg/kg. Blood samples are drawn at 2, 4, 6 and 10 minutes after giving glucose. The control group of animals receives the same vehicle along with glucose, but with no compound added. In some instances, a 30 minute post-glucose blood sample is drawn. Aprotinin is added to the blood sample (250 kIU/ml blood). The serum is then analyzed for glucose and insulin using standard methodologies.

The assay uses a formulated and calibrated peptide stock in PBS. Normally, this stock is a prediluted 100 μM stock. However, a more concentrated stock with approximately 1 mg agonist per mL is used. The specific concentration is always known. Variability in the maximal response is mostly due to variability in the vehicle dose.

Protocol details are as follows:

| SPECIES/STRAIN/WEIGHT | Rat/Wistar Unilever/approximately 275-300 g |
|---|---|
| TREATMENT DURATION | Single dose |
| DOSE VOLUME/ROUTE | 1.5 mL/kg/iv |
| VEHICLE | 8% PEG300, 0.1% BSA in water |
| FOOD/WATER REGIMEN | Rats are fasted overnight prior to surgery. |
| LIVE-PHASE PARAMETERS | Animals are sacrificed at the end of the test. |
| IVGTT: Performed on rats (with two | Glucose IV bolus: 500 mg/kg as 10% |
| catheters, jugular vein and carotid | solution (5 mL/kg) at time = 0. |
| artery) of each group, under | Compound iv: Just after glucose. |
| pentobarbital anesthesia. | Blood samplings (300 μL from carotid artery; |
| | EDTA as anticoagulant; aprotinin and PMSF |
| | as antiproteolytics; kept on ice): 0, 2, 4, 6, and |
| | 10 minutes. |
| | Parameter determined: Insulin. |
| TOXICOKINETICS | Plasma samples remaining after insulin |
| | measurements are kept at −20° C. and |
| | compound levels are determined. |
| NUMBER OF SAMPLES | 150 |

Delayed IVGTT: Perform IVGTT as described above, making the following changes. After the initial blood sample, compound or vehicle is injected i.v. Glucose is injected i.v. 30 minutes later in a separate injection. Blood samples are taken immediately prior to administration of the compound, at 15 minutes after administration of the compound, and at 30 minutes after administration of the compound. The sample at 30 minutes after administration of the compound is taken immediately prior to glucose administration. Blood samples are drawn 2, 4, 6, 10, and 30 minutes after giving glucose (i.e. 32, 34, 36, 40 and 60 minutes after compound administration). The blood samples at 15 and 60 minutes are not essential to the study and not always taken. Aprotinin is added to the blood sample (250 kIU/ml blood). The serum is then analyzed for glucose and insulin using standard methodologies.

The assay uses a formulated and calibrated peptide stock in PBS. Normally, this stock is a prediluted 100 μM stock. However, a more concentrated stock with approximately 1 mg agonist per mL is used. The specific concentration is always known.

Oral Glucose Tolerance Test (OGTT):

The effect of a selective VPAC2 receptor peptide agonist on plasma insulin and glucose is evaluated during OGTT in conscious Wistar rats. The maximal dose of agonist is 10 μg/kg. Since the peptide is given intravenously and has a very short half-life, a delay between glucose and compound administrations is applied.

Protocol details are as follows:

| SPECIES/STRAIN/ | Rat/Wistar Unilever/approximately 275-300 g | | | | |
|---|---|---|---|---|---|
| WEIGHT | | | | | |
| TREATMENT | Single dose | | | | |
| DURATION | | | | | |
| GROUP/COMPOUND/ | | | | | |
| DOSE/NUMBER/ | | | Dose | Injection Time | |
| SEX | Group | Compound | (μg/kg) | (min) | Number/Sex |
| | 1 | vehicle | 0 | 15 | 6M |
| | 2 | vehicle | 0 | 30 | 6M |
| | 5 | peptide | 10 | 15 | 6M |
| | 6 | peptide | 10 | 30 | 6M |

| | |
|---|---|
| DOSE | 1.5 mL/kg/iv |
| VOLUME/ROUTE | |
| VEHICLE | 8% PEG300, 0.1% BSA in water. |
| FOOD/WATER | Fasted overnight prior to the test. |
| REGIMEN | |
| LIVE-PHASE | Animals will be trained for contention, gavage, and tail |
| PARAMETERS | massage 2 days before the experiment. Animals will be |
| | sacrificed at the end of the test. Two animals of each group |
| | are tested on each day. |
| OGTT: Performed on | Glucose orally: 2.5 g/kg as 50% solution (5 mL/kg) at time = 0. |
| conscious, non- | Compound IV: 15 or 30 min after glucose. |
| cannulated rats of each | Blood samplings: (300 μL from tail tip; EDTA as |
| group. | anticoagulant; aprotinin and PMSF as antiproteolytics; kept on |
| | ice): Groups 1, 3, and 5: before glucose (time 0), at 15 min |
| | (just before compound), and at 20, 30, 45, 75, 105, and 135 |
| | minutes. |
| | Groups 2, 4, and 6: before glucose (time 0), at 30 min (just |
| | before compound), and at 35, 45, 60, 90, 120, and 150 |
| | minutes. |
| | Parameters determined: Insulin, glucose |
| TOXICOKINETICS | Plasma samples remaining after insulin measurements are kept |
| | at −20° C. and compound levels are determined. |

### EXAMPLE 9

### Pharmacokinetic Assay

An analysis of active peptide levels in rat plasma is conducted after IV injection of 10 μg/kg of each peptide. An IVGTT with glucose is given immediately after T=0 to 6 animals per condition. Samples are taken at 0, 2, 4, 6, and 10 minutes after injection.

For cell handling, 50,000 cells/well are plated and kept in culture over night. Cells are washed twice in PBS and 50 μl/well stimulation medium consisting of PBS+1.2 MgCl₂, 1.3 CaCl₂, 2 glucose and 0.5 IBMX is added. The plate is incubated for 15 minutes and 50 μl/well of the plasma samples is added. The plate is incubated for 30 minutes, the supernatant is removed and normal procedure with the DiscoveRx assay is followed. Plates are prepared in duplicate. Protease and peptidase inhibitor are present in all plasma samples.

### EXAMPLE 10

### DPP-IV HPLC Assays

Part 1: Formulation of Selective VPAC2 Receptor Peptide Agonists:

Approximately 2 mg of lyophilized peptide is weighed and dissolved in approximately 1.6 mL de-ionized water. If the peptide does not dissolve, the pH is adjusted with 1M NaOH to between pH 10.0 and 10.5. After incubation at room temperature for 30 minutes, 1/10^{th} of the original volume 10×PBS is added. The pH is adjusted to between pH 7.2 and 7.6. The peptide solution is filtered through a 0.22 μm Millex-GV syringe filter (Millipore, Bedford Mass., USA). The peptide concentration is determined through absorption at 280 nm. The peptide concentration is then adjusted to 100 μM. The peptides are frozen at −20° C. for further use.

Part 2: In Vitro Incubation of Selective VPAC2 Receptor Peptide Agonists with Purified Dipeptidyl-Peptidase IV (DPP-IV):

The stability of selective VPAC2 receptor peptide agonists against proteolysis by DPP-IV is determined using 100 μL of a 100 μM peptide solution in 1×PBS. A 10 μL solution is removed and quenched with 40 μL of 0.1% trifluoroacetic acid (TFA)/20% acetonitrile (ACN). This solution (20 μL) is analyzed by reversed-phase HPLC. The reversed-phase analysis consists of a Zorbax 300SB-C8 column (3.5 micron, 4.6×50 mm, Alltech Associates, Inc., Deerfield Ill., USA) running a 15-40% B gradient over 15 minutes at 60° C. where A-buffer is 0.1% (v/v) TFA in water and B-buffer is 0.085% (v/v) TFA in ACN. The peak area is integrated. This peak area serves as an internal control as 100% intact peptide.

A 10 μL aliquot of a 1.12 mU/μL solution of DPP-IV (Sigma, St. Louis, LO, USA) is added to 90 μL of a 100 μM solution of peptide, resulting in a substrate concentration of 90 μM peptide. The reaction mixture is then stored at 37° C. At various time-points, 10 μL of solution is removed, quenched with 40 μL 0.1% TFA/20% ACN, and analyzed by reversed-phase HPLC as described above. The remaining full length peptide concentration (nM) at each timepoint, except time=0, is calculated using following formula:

peak area [time x]*concentration [t0]/peak area [time 0]*0.9

For the time=0 timepoint, the concentration (nM) is calculated using the following formula:

$$\frac{peak area [ time x ] * initial substrate concentration [ 9 nM ]}{peak area [ time 0 ]}$$

Other modifications of the present invention will be apparent to those skilled in the art without departing from the scope of the invention.

1. A VPAC2 receptor peptide agonist comprising an amino acid sequence selected from the group consisting of:
SEQ ID NO: 17
HSDAVFTEQY(OMe)TRAibRAibQLAAAibOrnY(OMe)LQSIK
AibOrn;
SEQ ID NO: 18
HSDAVFTEK(CO(CH₂)₂SH)Y(OMe)TOrnLRAibQVAAAibOrn
YLQSIOrnOrn;
SEQ ID NO: 19
HSDAVFTEQY(OMe)TOrnLRAibQVAAAibOrnYLQSIOrnK(W)
Orn;
SEQ ID NO: 20
HSDAVFTEQY(OMe)TOrnLRAibQVAAAibK(CO(CH₂)₂SH)YLQ
SIOrnOrn;
SEQ ID NO: 21
HSDAVFTEQY(OMe)TOrnLRAibQVAAK(CO(CH₂)₂SH)OrnYLQ
SIOrnOrn;
SEQ ID NO: 22
HSDAVFTEQY(OMe)TOrnLRAibQVCAAibOrnYLQSIOrnOrn;
SEQ ID NO: 23
HSDAVFTEQY(OMe)TOrnLRCQVAAAibOrnYLQSIOrnOrn;
SEQ ID NO: 24
HSDAVFTEQY(OMe)TOrnLRAibQLAAAibOrnYLQSIOrnOrn;
SEQ ID NO: 25
HSDAVFTEQY(OMe)TOrnLRAibQVAAAibOrnYAibQSIOrnOrn;
SEQ ID NO: 26
HSDAVFTEQY(OMe)TOrnLRAibQVAAAibOrnYLQAibIOrnOrn;
SEQ ID NO: 27
HSDAVFTEQY(OMe)TOrnLRAibQVAAbuAibOrnYLQAibIOrnOrn;
SEQ ID NO: 28
HSDAVFTEQY(OMe)TOrnLRAibQLAAAibOrnYLQAibIOrnOrn;
SEQ ID NO: 29
HSDAVFTEQY(OMe)TOrnLRAibQLAAAibOrnYAibQAibIOrnOrn;
SEQ ID NO: 30
HSDAVFTEQY(OMe)TOrnLRAibQLAAbuAibOrnYAibQSIOrnOrn;
SEQ ID NO: 31
HSDAVFTEQY(OMe)TOrnLRAibQLAAbuAibOrnYLQSIOrnOrn;
SEQ ID NO: 32
HSDAVFTEQY(OMe)TOrnLRAibQLAAbuAibOrnYAibQAibIOrn
Orn;
SEQ ID NO: 33
HSDAVFTEQY(OMe)TOrnLRAibQLAAAibOrnYAibQSIOrnOrn;
SEQ ID NO: 34
HSDAVFTEQY(OMe)TOrnLRK(W)QVAAAibOrnYLQSIOrnOrn;
SEQ ID NO: 35
HSDAVFTEQY(OMe)TOrnLRAibQVAAAibOrnYLK(W)SIOrnOrn;
SEQ ID NO: 36
HSDAVFTEQY(OMe)TOrnLRAibQK(W)AAAibOrnYLQSIOrnOrn;
SEQ ID NO: 37
HSDAVFTEQY(OMe)TOrnLRK(CO(CH₂)₂SH)QVAAAibOrnYLQ
SIOrnOrn:
SEQ ID NO: 38
HSDAVFTEQY(OMe)TOrnLRAibQVAAAibK(W)YLQSIOrnOrn;
SEQ ID NO: 39
HSDAVFTEQY(OMe)TOrnLRAibQVAAAibCYLQSIOrnOrn;
SEQ ID NO: 40
HSDAVFTEQY(OMe)TOrnLRAibQLAAbuAibOrnYLQAibIOrnOrn;
SEQ ID NO: 41
HSDAVFTEQY(OMe)TOrnLRAibQVAAAibOrnYLQSK(W)OrnOrn;
SEQ ID NO: 42
HSDAVFTEQY(OMe)TOrnLRAibQLAAbuAibOrnYLQAibIOrnC
Orn;
SEQ ID NO: 43
HSDAVFTEQY(OMe)TOrnLRAibQLAAbuAibOrnYLQAibCOrn
Orn;
SEQ ID NO: 44
HSDAVFTEQY(OMe)TOrnLRAibQCAAbuAibOrnYLQAibIOrnOrn;
SEQ ID NO: 45
HSDAVFTEQY(OMe)TOrnLRCQLAAbuAibOrnYLQAibIOrnOrn;
SEQ ID NO: 92
HSDAVFTEQY(OMe)TOrnLRAibQVK(CO(CH₂)₂SH)AAibOrn
YLQSIOrnOrn;
SEQ ID NO: 93
HSDAVFTEQY(OMe)TOrnLRAibQLAAbuAibOrnYLQSIOrnCOrn;
SEQ ID NO: 94
HSDAVFTEQY(OMe)TOrnLRAibQLAAbuAibOrnYLQSCOrnOrn;
SEQ ID NO: 95
HSDAVFTEQY(OMe)TOrnLRAibQLAAbuAibOrnYLQAibIOrn
K(CO(CH₂)₂SH)Orn;
SEQ ID NO: 96
HSDAVFTEQY(OMe)TOrnLRAibQLAAbuAibOrnYLQSIOrn
K(CO(CH₂)₂SH)Orn;
SEQ ID NO: 97
HSDAVFTEQY(OMe)TOrnLRK(W)QLAAbuAibOrnYLQAibIOrn
Orn;
SEQ ID NO: 98
HSDAVFTEQY(OMe)TOrnLRAibQLAAAibOrnYLQSIOrnOrnC;
SEQ ID NO: 99
HSDAVFTEQY(OMe)TOrnLRAibQVAAAibOrnYLQSIOrnOrnC;
SEQ ID NO: 100
HSDAVFTEQY(OMe)TOrnLRAibQLAAbuAibOrnYLQSIOrnOrnC;
SEQ ID NO: 101
HSDAVFTEQY(OMe)TOrnLRAibQLAAbuAibOrnY(OMe)LQAibI
OrnOrn;
SEQ ID NO: 102
HSDAVFTEQY(OMe)TOrnLRAibQLAAbuAibOrnY(OMe)LQAibI
OrnCOrn;
SEQ ID NO: 103
HSDAVFTEQY(OMe)TOrnLRAibQCAAbuAibOrnY(OMe)LQAibI
OrnOrn;
SEQ ID NO: 104
HSDAVFTEQY(OMe)TOrnLRAibQLAAbuAibOrnYLQAibIOrn
OrnC;
SEQ ID NO: 105
HSDAVFTEQY(OMe)TOrnLRAibQLAAbuAibOrnY(OMe)LQSI
OrnOrn;
SEQ ID NO: 106
HSDAVFTEQY(OMe)TOrnLRAibQCAAbuAibOrnY(OMe)LQSI
OrnOrn;
SEQ ID NO: 107
HSDAVFTEQY(OMe)TOrnLRAibQLAAbuAibOrnY(OMe)LQSI
OrnCOrn;
SEQ ID NO: 108
HSDAVFTEQY(OMe)TOrnLRAibQLAbuAAibOrnYLQSIOrnOrn;
SEQ ID NO: 109
HSDAVFTEQY(OMe)TOrnLRAibQK(CO(CH₂)₂SH)AAbu
AibOrnYLQAibIOrnOrn;
SEQ ID NO: 110
HSDAVFTEQY(OMe)TOrnLRAibQK(W)AAbuAibOrnYLQ
AibIOrnOrn;
SEQ ID NO: 145
HSDAVFTDNYTRLRKQVAAKKYLQSIKNKRQ;
SEQ ID NO: 146
HSDAVFTDNYTLLRAibQVAAAibKYLQSIOrnNOrn;
SEQ ID NO: 147
HSDAVFTDNYTQLRAibQVAAAibKYLQSIOrnNOrn;
SEQ ID NO: 148
HSDAVFTDNYTFLRAibQVAAAibKYLQSIOrnNOrn;
SEQ ID NO: 149
HSDAVFTDNYTOrnLRAibQVAAAibKYLQCIOrnNOrn;
SEQ ID NO: 150
HSDAVFTDNYTOrnLRAibQVAACOrnYLQSIOrnNOrn;
SEQ ID NO: 151
HSDAVFTDNYTOrnLRAibQVAAAibKYLQSSOrnNOrn;
SEQ ID NO: 152
HSDAVFTDNYTOrnLRAibQVAAAibKYLSSIOrnNOrn;
SEQ ID NO: 153
HSDAVFTDNYTOrnLRAibQVAAAibKYSQSIOrnNOrn;
SEQ ID NO: 154
HSDAVFTDNYTOrnLRAibQVAAAibKSLQSIOrnNOrn;
SEQ ID NO: 155
HSDAVFTDNYTOrnLRAibQVAAAibSYLQSIOrnNOrn;
SEQ ID NO: 156
HSDAVFTDNYTOrnLRAibQVSAAibKYLQSIOrnNOrn;
SEQ ID NO: 157
HSDAVFTDNYTOrnLRAibQSAAAibKYLQSIOrnNOrn;
SEQ ID NO: 158
HSDAVFTDNYTOrnLRAibSVAAAibKYLQSIOrnNOrn;
SEQ ID NO: 159
HSDAVFTDNYTOrnSRAibQVAAAibKYLQSIOrnNOrn;
SEQ ID NO: 160
HSDAVFTDSYTOrnLRAibQVAAAibKYLQSIOrnNOrn;
SEQ ID NO: 161
HSDAVFTDNYThRLRAibQVAAAibKYLQSIKNKRY;
SEQ ID NO: 162
HSDAVFTDNYTRLRAibQVAAAibKYLQSIKAibOrn;
SEQ ID NO: 163
HSDAVFTDNY(OMe)TRLRAibQVAAAibKYLQSIKNKRY;
SEQ ID NO: 164
HSEAVFTENYTOrnLRAibQVAAAibKYLQSIOrnNOrn;
SEQ ID NO: 165
HSDAVFTDQYTOrnLRAibQVAAAibKYLQSIOrnQOrn;
SEQ ID NO: 166
HSDAVFTDNYTRLLAKLALQKYLQSIOrnNOrn;
SEQ ID NO: 167
HSDAVFTDNYTOrnLLAKLALQKYLQSIOrnNOrn;
SEQ ID NO: 168
HSEAVFTEQYTOrnLRAibQVAAAibOrnYLQSIOrnOrn;
SEQ ID NO: 169
HSDAVFTDNYTOrnLRAibQVASAibKYLQSIOrnNOrn;
SEQ ID NO: 170
HSEAVFTEQY(OMe)TOrnLRAibQLAAAibOrnYLQSIOrnOrn;
SEQ ID NO: 171
HSDAVFTDQY(OMe)TOrnLRAibQLAAAibOrnYLQSIOrnOrn;
SEQ ID NO: 172
HSDAVFTDQYTOrnLRAibQLAAAibOrnYLQSIOrnOrn;
SEQ ID NO: 173
HSDAVFTDQYTOrnLRAibQVAAAibOrnYLQSIOrnOrn;
SEQ ID NO: 174
HSDAVFTDNYTOrnLRAibQVAAAibOrnYLQSIOrnOrn;
SEQ ID NO: 175
HSDAVFTEQY(OMe)TOrnLRAibQVAAAibOrnYLQSIOrnOrn;
SEQ ID NO: 176
HSDAVFTDNYTRAibRAibQVAAAibKYLQSIKAibK;
SEQ ID NO: 177
HSDAVFTDQYTRAibRAibQVAAAibKYLQSIKAibK;
SEQ ID NO: 178
HSDAVFTDQYTRAibRAibQLAAAibKYLQSIKAibK;
SEQ ID NO: 179
HSDAVFTDQY(OMe)TRAibRAibQLAAAibKYLQSIKAibK;
SEQ ID NO: 180
HSEAVFTEQY(OMe)TRAibRAibQLAAAibKYLQSIKAibK;
SEQ ID NO: 181
HSDAVFTEQY(OMe)TOrnLRAibQVAAAibOrnYLCSIOrnOrn;
SEQ ID NO: 182
HSDAVFTEQY(OMe)TOrnLRAibQVAAAibOrnYL
K(CO(CH₂)₂SH)SIOrnOrn;
SEQ ID NO: 183
HSDAVFTEQY(OMe)TOrnLRAibQVACAibOrnYLQSIOrnOrn;
SEQ ID NO: 184
HSDAVFTEQY(OMe)TOrnLRAibQVAK(CO(CH₂)₂SH)AibOrn
YLQSIOrnOrn;
SEQ ID NO: 185
HSDAVFTEQY(OMe)TOrnLRAibCVAAAibOrnYLQSIOrnOrn;
SEQ ID NO: 186
HSDAVFTDNYTOrnLRK(W)QVAAAibKYLQSIOrnNOrn;
SEQ ID NO: 187
HSDAVFTEQY(OMe)TOrnLRAibQLAAAibOrnY(OMe)LQ
SIOrnOrn;
SEQ ID NO: 188
HSDAVFTEQY(OMe)TOrnLRAibQVAAAibOrnYLQSCOrnOrn;
SEQ ID NO: 189
HSDAVFTEQY(OMe)TOrnLRAibQCAAAibOrnYLQSIOrnOrn;
SEQ ID NO: 190
HSDAVFTEQY(OMe)TOrnLRAibQVAAAibOrnYLQSIOrnCOrn;
SEQ ID NO: 191
HSDAVFTEQY(OMe)TOrnLRAibQVAAAibOrnYLQCIOrnOrn;
SEQ ID NO: 192
HSDAVFTECY(OMe)TOrnLRAibQVAAAibOrnYLQSIOrnOrn;
SEQ ID NO: 193
HSDAVFTEQY(OMe)TOrnCRAibQVAAAibOrnYLQSIOrnOrn;
SEQ ID NO: 194
HSDAVFTEQY(OMe)TOrnLRAibQVAAAibOrnYLQS
K(CO(CH₂)₂SH)OrnOrn;
SEQ ID NO: 195
HSDAVFTEQY(OMe)TOrnLRAibQVAAAibOrnYLQ
K(CO(CH₂)₂SH)IOrnOrn;
SEQ ID NO: 196
HSDAVFTEQY(OMe)TOrnLRAibQK(CO(CH₂)₂SH)AAAibOrnYL
QSIOrnOrn;
SEQ ID NO: 197
HSDAVFTEQY(OMe)TOrnLRAibK(CO(CH₂)₂SH)VAAAibOrnY
LQSIOrnOrn;
SEQ ID NO: 199
HSDAVFTEQY(OMe)TOrnK(CO(CH₂)₂SH)RAibQVAAAibOrn
YLQSIOrnOrn;
and a C-terminal extensions wherein the N-terminus of said C-terminal extension is linked to the C-terminus of said peptide and wherein said C-terminal extension comprises an amino acid sequence of the formula:
Formula 3
(SEQ ID NO: 3)
Xaa₁-Xaa₂-Xaa₃-Xaa₄-Xaa₅-Xaa₆-Xaa₇-Xaa₈-Xaa₉-
Xaa₁₀-Xaa₁₁-Xaa₁₂
wherein:
Xaa₁ is: Gly, Cys, or absent;
Xaa₂ is: Gly, Arg, or absent;
Xaa₃ is: Pro, Thr, or absent;
Xaa₄ is: Ser, or absent;
Xaa₅ is: Ser, or absent;
Xaa₆ is: Gly, or absent;
Xaa₇ is: Ala, or absent;
Xaa₈ is: Pro, or absent;
Xaa₉ is: Pro, or absent;
Xaa₁₀ is: Pro, or absent;
Xaa₁₁ is: Ser, Cys, or absent; and
Xaa₁₂ is: Cys, or absent;
wherein at least five of Xaa₁ to Xaa₁₂ of said C-terminal extension are present and wherein if Xaa₁, Xaa₂, Xaa₃, Xaa₄, Xaa₅, Xaa₆, Xaa₇, Xaa₈, Xaa₉, Xaa₁₀, or Xaa₁₁ is absent, the next amino acid present downstream is the next amino acid in said C-terminal extension and wherein the C-terminal amino acid may be amidated.

2. A VPAC2 receptor peptide agonist according to claim 1, wherein said C-terminal extension is selected from the group consisting of:

| | |
|---|---|
| SEQ ID NO: 5 | GGPSSGAPPPS |
| SEQ ID NO: 6 | GGPSSGAPPPS-NH₂ |
| SEQ ID NO: 7 | GGPSSGAPPPC |
| SEQ ID NO: 8 | GGPSSGAPPPC-NH₂ |
| SEQ ID NO: 9 | GRPSSGAPPPS |
| SEQ ID NO: 10 | GRPSSGAPPPS-NH₂ |
| SEQ ID NO: 11 | GGPSSGAPPPCC |
| SEQ ID NO: 12 | GGPSSGAPPPCC-NH₂ |

3. A VPAC2 receptor peptide agonist according to claim 1 or 2, further comprising an N-terminal modification at the N-terminus of said peptide agonist wherein said N-terminal modification is selected from the group consisting of:
(a) addition of D-histidine, isoleucine, methionine, or norleucine;
(b) addition of a peptide comprising the amino acid sequence Ser-Trp-Cys-Glu-Pro-Gly-Trp-Cys-Arg (SEQ ID NO: 91) wherein said Arg is linked to the N-terminus of said peptide agonist;
(c) addition of C₁-C₁₆ alkyl optionally substituted with one or more substituents independently selected from aryl, C₁-C₆ alkoxy, —NH₂, —OH, halogen and —CF₃;
(d) addition of —C(O)R¹ wherein R¹ is a C₁-C₁₆ alkyl optionally substituted with one or more substituents independently selected from aryl, C₁-C₆ alkoxy, —NH₂, —OH, halogen, —SH and —CF₃; an aryl optionally substituted with one or more substituents independently selected from C₁-C₆ alkyl, C₂-C₆ alkenyl, C₂-C₆ alkynyl, C₁-C₆ alkoxy, —NH₂, —OH, halogen and —CF₃; an aryl C₁-C₄ alkyl optionally substituted with one or more substituents independently selected from C₁-C₆ alkyl, C₂-C₆ alkenyl, C₂-C₆ alkynyl, C₁-C₆ alkoxy, —NH₂, —OH, halogen and —CF₃; —NR²R³ wherein R² and R³ are independently hydrogen, C₁-C₆ alkyl, aryl or aryl C₁-C₄ alkyl; —OR⁴ wherein R⁴ is C₁-C₁₆ alkyl optionally substituted with one or more substituents independently selected from aryl, C₁-C₆ alkoxy, —NH₂, —OH, halogen and —CF₃, aryl optionally substituted with one or more substituents independently selected from C₁-C₆ alkyl, C₂-C₆ alkenyl, C₂-C₆ alkynyl, C₁-C₆ alkoxy, —NH₂, —OH, halogen and —CF₃, or aryl C₁-C₄ alkyl optionally substituted with one or more substituents independently selected from C₁-C₆ alkyl, C₂-C₆ alkenyl, C₂-C₆ alkynyl, C₁-C₆ alkoxy, —NH₂, —OH, halogen and —CF₃; or 5-pyrrolidin-2-one;
(e) addition of —SO₂R⁵ wherein R⁵ is aryl, aryl C₁-C₄ alkyl or C₁-C₁₆ alkyl;
(f) formation of a succinimide group optionally substituted with C₁-C₆ alkyl or —SR⁶, wherein R⁶ is hydrogen or C₁-C₆ alkyl;
(g) addition of methionine sulfoxide;
(h) addition of biotinyl-6-aminohexanoic acid (6-aminocaproic acid); and
(i) addition of —C(═NH)—NH₂.

4. A VPAC2 receptor peptide agonist according to claim 3, further comprising an N-terminal modification wherein said N-terminal modification is the addition of a group selected from the group consisting of acetyl, propionyl, butyryl, pentanoyl, hexanoyl, methionine, methionine sulfoxide, 3-phenylpropionyl, phenylacetyl, benzoyl, norleucine, D-histidine, isoleucine, 3-mercaptopropionyl, biotinyl-6-aminohexanoic acid (6-aminocaproic acid), and —C(═NH)—NH₂.

5. A VPAC2 receptor peptide agonist according to claim 4, wherein said N-terminal modification is the addition of acetyl or hexanoyl.

6. A VPAC2 peptide receptor agonist according to claim 1, comprising an amino acid sequence selected from the group consisting of:

| | |
|---|---|
| Agonist # | Sequence |
| | |
|---|---|
| P400 | C6- |
| | HSDAVFTEQY(OMe)TOrnLRAibQVAAAibK(W)YLQSIOrnOrnGGPSSGA |
| | PPPS-NH₂ |
| P416 | C6- |
| | HSDAVFTEQY(OMe)TRAibRAibQLAAAibOrnY(OMe)LQSIKAibOrnGGP |
| | SSGAPPPC-NH₂ |
| P450 | C6- |
| | HSDAVFTEK(CO(CH₂)₂SH)Y(OMe)TOrnLRAibQVAAAibOrnYLQSIOrn |
| | OrnGGPSSGAPPPS-NH₂ |
| P453 | C6- |
| | HSDAVFTEQY(OMe)TOrnLRAibQVAAAibOrnYLQSIOrnK(W)OrnGGPSS |
| | GAPPPS-NH₂ |
| P459 | C6- |
| | HSDAVFTEQY(OMe)TOrnLRAibQVAAAibCYLQSIOrnOrnGGPSSGAPPP |
| | S-NH₂ |
| P471 | C6- |
| | HSDAVFTEQY(OMe)TOrnLRAibQVAAAibK(CO(CH₂)₂SH)YLQSIOrnOrn |
| | GGPSSGAPPPS-NH₂ |
| P474 | C6- |
| | HSDAVFTEQY(OMe)TOrnLRAibQVAAK(CO(CH₂)₂SH)OrnYLQSIOrnOrn |
| | GGPSSGAPPPS-NH₂ |
| P477 | C6- |
| | HSDAVFTEQY(OMe)TOrnLRAibQVCAAibOrnYLQSIOrnOrnGGPSSGAPP |
| | PS-NH₂ |
| P482 | C6- |
| | HSDAVFTEQY(OMe)TOrnLRCQVAAAibOrnYLQSIOrnOrnGGPSSGAPPP |
| | S-NH₂ |
| P487 | C6- |
| | HSDAVFTEQY(OMe)TOrnLRAibQLAAAibOrnYLQSIOrnOrnGGPSSGAPP |
| | PS-NH₂ |
| P506 | C6- |
| | HSDAVFTEQY(OMe)TOrnLRAibQVAAAibOrnYAibQSIOrnOrnGGPSSGA |
| | PPPC-NH₂ |
| P508 | C6- |
| | HSDAVFTEQY(OMe)TOrnLRAibQVAAAibOrnYLQAibIOrnOrnGGPSSGA |
| | PPPC-NH₂ |
| P510 | C6- |
| | HSDAVFTEQY(OMe)TOrnLRAibQVAAbuAibOrnYLQAibIOrnOrnGGPSSG |
| | APPPC-NH₂ |
| P514 | C6- |
| | HSDAVFTEQY(OMe)TOrnLRAibQLAAAibOrnYLQAibIOrnOrnGGPSSGA |
| | PPPCC-NH₂ |
| P516 | C6- |
| | HSDAVFTEQY(OMe)TOrnLRAibQLAAbuAibOrnYLQAibIOrnOrnGGPSSG |
| | APPPCC-NH₂ |
| P518 | C6- |
| | HSDAVFTEQY(OMe)TOrnLRAibQLAAAibOrnYAibQAibIOrnOrnGGPSSG |
| | APPPCC-NH₂ |
| P520 | C6- |
| | HSDAVFTEQY(OMe)TOrnLRAibQLAAbuAibOrnYAibQSIOrnOrnGGPSSG |
| | APPPCC-NH₂ |
| P522 | C6- |
| | HSDAVFTEQY(OMe)TOrnLRAibQLAAbuAibOrnYLQSIOrnOrnGGPSSGA |
| | PPPCC-NH₂ |
| P524 | C6- |
| | HSDAVFTEQY(OMe)TOrnLRAibQLAAbuAibOrnYAibQAibIOrnOrnGGPS |
| | SGAPPPCC-NH₂ |
| P526 | C6- |
| | HSDAVFTEQY(OMe)TOrnLRAibQLAAAibOrnYAibQSIOrnOrnGGPSSGA |
| | PPPCC-NH₂ |
| P528 | C6- |
| | HSDAVFTEQY(OMe)TOrnLRK(W)QVAAAibOrnYLQSIOrnOrnGGPSSGA |
| | PPPS-NH₂ |
| P530 | C6- |
| | HSDAVFTEQY(OMe)TOrnLRAibQVAAAibOrnYLK(W)SIOrnOrnGGPSSG |
| | APPPS-NH₂ |
| P532 | C6- |
| | HSDAVFTEQY(OMe)TOrnLRAibQK(W)AAAibOrnYLQSIOrnOrnGGPSSG |
| | APPPS-NH₂ |
| P534 | C6- |
| | HSDAVFTEQY(OMe)TOrnLRAibQVAAAibOrnYLQSK(W)OrnOrnGGPSS |
| | GAPPPS-NH₂ |
| P536 | C6- |
| | HSDAVFTEQY(OMe)TOrnLRAibQLAAbuAibOrnYLQAibIOrnCOrnGGPSS |
| | GAPPPS-NH₂ |
| P540 | C6- |
| | HSDAVFTEQY(OMe)TOrnLRAibQLAAbuAibOrnYLQAibCOrnOrnGGPSS |
| | GAPPPS-NH₂ |
| P544 | C6- |
| | HSDAVFTEQY(OMe)TOrnLRAibQCAAbuAibOrnYLQAibIOrnOrnGGPSSG |
| | APPPS-NH₂ |
| P546 | C6- |
| | HSDAVFTEQY(OMe)TOrnLRCQLAAbuAibOrnYLQAibIOrnOrnGGPSSGA |
| | PPPS-NH₂ |
| P488 | C6- |
| | HSDAVFTEQY(OMe)TOrnLRAibQVAAAibOrnYLQSIOrnOrnGGPSSGAPP |
| | PS-NH₂ |
| P489 | C6- |
| | HSDAVFTEQY(OMe)TOrnLRAibQVAAAibOrnYLK(CO(CH₂)₂SH)SIOrn |
| | OrnGGPSSGAPPPC-NH₂ |
| P491 | C6- |
| | HSDAVFTEQY(OMe)TOrnLRAibQVAK(CO(CH₂)₂SH)AibOrnYLQSIOrn |
| | OrnGGPSSGAPPPC-NH₂ |
| P494 | C6- |
| | HSDAVFTEQY(OMe)TOrnLRAibQVAAAibOrnYLQK(CO(CH₂)₂SH)IOrn |
| | OrnGGPSSGAPPPC-NH₂ |
| P496 | C6- |
| | HSDAVFTEQY(OMe)TOrnLRAibQVAAAibOrnYLQSK(CO(CH₂)₂SH)Orn |
| | OrnGGPSSGAPPPC-NH₂ |
| P498 | C6-HSDAVFTEQY(OMe)TOrnLRAibQVAAAibOrnYLQSIOrn |
| | K(CO(CH₂)₂SH)OrnGGPSSGAPPPC-NH₂ |
| P500 | C6- |
| | HSDAVFTEQY(OMe)TOrnLRAibQVAAAibOrnYLCSIOrnOrnGGPSSGAPP |
| | PC-NH₂ |
| P502 | C6- |
| | HSDAVFTEQY(OMe)TOrnLRAibQVAAAibOrnYLQSCOrnOrnGGPSSGAP |
| | PPC-NH₂ |
| P504 | C6- |
| | HSDAVFTEQY(OMe)TOrnLRAibQVAAAibOrnYLQSIOrnCOrnGGPSSGAP |
| | PPC-NH₂ |
| P479 | C6- |
| | HSDAVFTEQY(OMe)TOrnLRAibQVK(CO(CH₂)₂SH)AAibOrnYLQSIOrn |
| | OrnGGPSSGAPPPS-NH₂ |
| P538 | C6- |
| | HSDAVFTEQY(OMe)TOrnLRAibQLAAbuAibOrnYLQSIOrnCOrnGGPSSG |
| | APPPS-NH₂ |
| P542 | C6- |
| | HSDAVFTEQY(OMe)TOrnLRAibQLAAbuAibOrnYLQSCOrnOrnGGPSSGA |
| | PPPS-NH₂ |
| P548 | C6-HSDAVFTEQY(OMe)TOrnLRAibQLAAbuAibOrnYLQAibIOrn |
| | K(CO(CH₂)₂SH)OrnGGPSSGAPPPC-NH₂ |
| P550 | C6-HSDAVFTEQY(OMe)TOrnLRAibQLAAbuAibOrnYLQSIOrn |
| | K(CO(CH₂)₂SH)OrnGGPSSGAPPPC-NH₂ |
| P554 | C6- |
| | HSDAVFTEQY(OMe)TOrnLRAibQLAAbuAibOrnYLQSIOrnCOrnGGPSSG |
| | APPPC-NH₂ |
| P556 | C6- |
| | HSDAVFTEQY(OMe)TOrnLRK(W)QLAAbuAibOrnYLQAibIOrnOrnGGPSS |
| | GAPPPS-NH₂ |
| P559 | C6- |
| | HSDAVFTEQY(OMe)TOrnLRAibQLAAAibOrnYLQSIOrnOrnCGGPSSGAP |
| | PPS-NH₂ |
| P561 | C6- |
| | HSDAVFTEQY(OMe)TOrnLRAibQVAAAibOrnYLQSIOrnOrnCGGPSSGAP |
| | PPC-NH₂ |
| P563 | C6- |
| | HSDAVFTEQY(OMe)TOrnLRAibQLAAbuAibOrnYLQAibIOrnOrnCGGPSS |
| | GAPPPS-NH₂ |
| P565 | C6- |
| | HSDAVFTEQY(OMe)TOrnLRAibQLAAbuAibOrnYLQSIOrnOrnCGGPSSG |
| | APPPS-NH₂ |
| P571 | C6- |
| | HSDAVFTEQY(OMe)TOrnLRAibQLAAbuAibOrnYLQSIOrnOrnCGGPSSG |
| | APPPC-NH₂ |
| P573 | C6- |
| | HSDAVFTEQY(OMe)TOrnLRAibQLAAbuAibOrnY(OMe)LQAibIOrnOrnG |
| | GPSSGAPPPCC-NH₂ |
| P575 | C6- |
| | HSDAVFTEQY(OMe)TOrnLRAibQLAAbuAibOrnY(OMe)LQAibIOrnCOrn |
| | GGPSSGAPPPS-NH₂ |
| P577 | C6- |
| | HSDAVFTEQY(OMe)TOrnLRAibQCAAbuAibOrnY(OMe)LQAibIOrnOrnG |
| | GPSSGAPPPS-NH₂ |
| P579 | C6- |
| | HSDAVFTEQY(OMe)TOrnLRAibQLAAbuAibOrnYLQAibIOrnOrnCGGPSS |
| | GAPPPC-NH₂ |
| P581 | C6- |
| | HSDAVFTEQY(OMe)TOrnLRAibQLAAbuAibOrnY(OMe)LQSIOrnOrnGGP |
| | SSGAPPPCC-NH₂ |
| P583 | C6- |
| | HSDAVFTEQY(OMe)TOrnLRAibQLAAbuAibOrnY(OMe)LQSIOrnCOrnGG |
| | PSSGAPPPS-NH₂ |
| P585 | C6- |
| | HSDAVFTEQY(OMe)TOrnLRAibQCAAbuAibOrnY(OMe)LQSIOrnOrnGGP |
| | SSGAPPPS-NH₂ |
| P587 | C6- |
| | HSDAVFTEQY(OMe)TOrnLRAibQLAAbuAibOrnY(OMe)LQSIOrnCOrnGG |
| | PSSGAPPPC-NH₂ |
| P589 | C6- |
| | HSDAVFTEQY(OMe)TOrnLRAibQLAbuAAibOrnYLQSIOrnOrnGGPSSGA |
| | PPPCC-NH₂ |
| P596 | C6- |
| | HSDAVFTEQY(OMe)TOrnLRAibQK(CO(CH₂)₂SH)AAbuAibOrnYLQAibI |
| | OrnOrnGGPSSGAPPPC-NH₂ |
| P598 | C6- |
| | HSDAVFTEQY(OMe)TOrnLRAibQK(CO(CH₂)₂SH)AAbuAibOrnYLQAibI |
| | OrnOrnGGPSSGAPPPS-NH₂ |
| P600 | C6- |
| | HSDAVFTEQY(OMe)TOrnLRAibQK(W)AAbuAibOrnYLQAibIOrnOrnGGP |
| | SSGAPPPS-NH₂ |
| P484 | C6- |
| | HSDAVFTEQY(OMe)TOrnLRK(CO(CH₂)₂SH)QVAAAibOrnYLQSIOrnOrn |
| | GGPSSGAPPPS-NH₂ |
| P552 | C6- |
| | HSDAVFTEQY(OMe)TOrnLRAibQLAAbuAibOrnYLQAibIOrnCOrnGGPSS |
| | GAPPPC-NH₂ |
| P567 | C6- |
| | HSDAVFTEQY(OMe)TOrnLRAibQCAAAibOrnYLQSIOrnOrnGGPSSGAPP |
| | PC-NH₂ |
| P591 | C6- |
| | HSDAVFTEQY(OMe)TOrnLRAibQLAAbuAibOrnYAibQAibIOrnOrnGGPS |
| | SGAPPPS-NH₂ |
| P594 | C6- |
| | HSDAVFTEQY(OMe)TOrnLRAibQCAAbuAibOrnYLQAibIOrnOrnGGPSSG |
| | APPPC-NH₂ |
| P29 | HSDAVFTDNYTRLRKQVAAKKYLQSIKNKRQGGPSSGAPPPS |
| P343 | C6-HSDAVFTDNYThRLRAibQVAAAibKYLQSIKNKRYGGPSSGAPPPS |
| P357 | C6-HSDAVFTDNYTRLRAibQVAAAibKYLQSIKAibOrnGGPSSGAPPPS |
| P358 | C6- |
| | HSDAVFTDNY(OMe)TRLRAibQVAAAibKYLQSIKNKRYGGPSSGAPPPS |
| P362 | C6-HSEAVFTENYTOrnLRAibQVAAAibKYLQSIOrnNOrnGGPSSGAPPPS |
| P363 | C6-HSDAVFTDQYTOrnLRAibQVAAAibKYLQSIOrnQOrnGGPSSGAPPPS |
| P367 | C6-HSDAVFTDNYTLLRAibQVAAAibKYLQSIOrnNOrnGGPSSGAPPPS |
| P368 | C6-HSDAVFTDNYTQLRAibQVAAAibKYLQSIOrnNOrnGGPSSGAPPPS |
| P369 | C6-HSDAVFTDNYTFLRAibQVAAAibKYLQSIOrnNOrnGGPSSGAPPPS |
| P370 | C6-HSDAVFTDNYTRLLAKLALQKYLQSIOrnNOrnGGPSSGAPPPS |
| P371 | C6-HSDAVFTDNYTOrnLLAKLALQKYLQSIOrnNOrnGGPSSGAPPPS |
| P372 | C6-HSDAVFTDNYTOrnLRAibQVAAAibKYLQCIOrnNOrnGGPSSGAPPPS |
| P377 | C6- |
| | HSDAVFTDNYTOrnLRAibQVAACOrnYLQSIOrnNOrnGGPSSGAPPPS- |
| | NH₂ |
| P379 | C6- |
| | HSEAVFTEQYTOrnLRAibQVAAAibOrnYLQSIOrnOrnGGPSSGAPPPC- |
| | NH₂ |
| P382 | C6-HSDAVFTDNYTOrnLRAibQVAAAibKYLQSSOrnNOrnGGPSSGAPPPS |
| P383 | C6-HSDAVFTDNYTOrnLRAibQVAAAibKYLSSIOrnNOrnGGPSSGAPPPS |
| P384 | C6-HSDAVFTDNYTOrnLRAibQVAAAibKYSQSIOrnNOrnGGPSSGAPPPS |
| P385 | C6-HSDAVFTDNYTOrnLRAibQVAAAibKSLQSIOrnNOrnGGPSSGAPPPS |
| P386 | C6-HSDAVFTDNYTOrnLRAibQVAAAibSYLQSIOrnNOrnGGPSSGAPPPS |
| P387 | C6-HSDAVFTDNYTOrnLRAibQVASAibKYLQSIOrnNOrnGGPSSGAPPPS |
| P388 | C6-HSDAVFTDNYTOrnLRAibQVSAAibKYLQSIOrnNOrnGGPSSGAPPPS |
| P389 | C6-HSDAVFTDNYTOrnLRAibQSAAAibKYLQSIOrnNOrnGGPSSGAPPPS |
| P390 | C6-HSDAVFTDNYTOrnLRAibSVAAAibKYLQSIOrnNOrnGGPSSGAPPPS |
| P391 | C6-HSDAVFTDNYTOrnSRAibQVAAAibKYLQSIOrnNOrnGGPSSGAPPPS |
| P392 | C6-HSDAVFTDSYTOrnLRAibQVAAAibKYLQSIOrnNOrnGGPSSGAPPPS |
| P393 | C6- |
| | HSEAVFTEQY(OMe)TOrnLRAibQLAAAibOrnYLQSIOrnOrnGGPSSGAPP |
| | PS |
| P394 | C6- |
| | HSDAVFTDQY(OMe)TOrnLRAibQLAAAibOrnYLQSIOrnOrnGGPSSGAPP |
| | PS |
| P395 | C6-HSDAVFTDQYTOrnLRAibQLAAAibOrnYLQSIOrnOrnGGPSSGAPPPS |
| P396 | C6-HSDAVFTDQYTOrnLRAibQVAAAibOrnYLQSIOrnOrnGGPSSGAPPPS |
| P397 | C6-HSDAVFTDNYTOrnLRAibQVAAAibOrnYLQSIOrnOrnGGPSSGAPPPS |
| P398 | C6- |
| | HSDAVFTEQY(OMe)TOrnLRAibQVAAAibOrnYLQSIOrnOrnGGPSSGAPP |
| | PC-NH₂ |
| P405 | C6-HSDAVFTDNYTRAibRAibQVAAAibKYLQSIKAibKGGPSSGAPPPS |
| P406 | C6-HSDAVFTDQYTRAibRAibQVAAAibKYLQSIKAibKGGPSSGAPPPS |
| P407 | C6-HSDAVFTDQYTRAibRAibQLAAAibKYLQSIKAibKGGPSSGAPPPS |
| P408 | C6- |
| | HSDAVFTDQY(OMe)TRAibRAibQLAAAibKYLQSIKAibKGGPSSGAPPPS |
| P409 | C6- |
| | HSEAVFTEQY(OMe)TRAibRAibQLAAAibKYLQSIKAibKGGPSSGAPPPS |
| P412 | C6- |
| | HSDAVFTDNYTOrnLRK(W)QVAAAibKYLQSIOrnNOrnGGPSSGAPPPS |
| P414 | C6- |
| | HSDAVFTEQY(OMe)TOrnLRAibQLAAAibOrnY(OMe)LQSIOrnOrnGGPSS |
| | GAPPPC-NH₂ |
| P418 | C6- |
| | HSDAVFTEQY(OMe)TOrnLRAibQLAAAibOrnY(OMe)LQSIOrnOrnGGPSS |
| | GAPPPS |
| P419 | C6- |
| | HSDAVFTEQY(OMe)TOrnLRAibQVAAAibOrnYLQSCOrnOrnGGPSSGAP |
| | PPS-NH₂ |
| P421 | C6- |
| | HSDAVFTEQY(OMe)TOrnLRAibQVAAAibOrnYLQSK(CO(CH₂)₂SH)Orn |
| | OrnGGPSSGAPPPS-NH₂ |
| P423 | C6- |
| | HSDAVFTEQY(OMe)TOrnLRAibQVAAAibOrnYLQK(CO(CH₂)₂SH)IOrn |
| | OrnGGPSSGAPPPS-NH₂ |
| P425 | C6- |
| | HSDAVFTEQY(OMe)TOrnLRAibQVAAAibOrnYLCSIOrnOrnGGPSSGAPP |
| | PS-NH₂ |
| P427 | C6- |
| | HSDAVFTEQY(OMe)TOrnLRAibQVAAAibOrnYLK(CO(CH₂)₂SH)SIOrn |
| | OrnGGPSSGAPPPS-NH₂ |
| P429 | C6- |
| | HSDAVFTEQY(OMe)TOrnLRAibQVACAibOrnYLQSIOrnOrnGGPSSGAPP |
| | PS-NH₂ |
| P431 | C6- |
| | HSDAVFTEQY(OMe)TOrnLRAibQVAK(CO(CH₂)₂SH)AibOrnYLQSIOrn |
| | OrnGGPSSGAPPPS-NH₂ |
| P433 | C6- |
| | HSDAVFTEQY(OMe)TOrnLRAibQCAAAibOrnYLQSIOrnOrnGGPSSGAPP |
| | PS-NH₂ |
| P435 | C6- |
| | HSDAVFTEQY(OMe)TOrnLRAibQK(CO(CH₂)₂SH)AAAibOrnYLQSIOrn |
| | OrnGGPSSGAPPPS-NH₂ |
| P437 | C6- |
| | HSDAVFTEQY(OMe)TOrnLRAibCVAAAibOrnYLQSIOrnOrnGGPSSGAPP |
| | PS-NH₂ |
| P439 | C6- |
| | HSDAVFTEQY(OMe)TOrnLRAibK(CO(CH₂)₂SH)VAAAibOrnYLQSIOrn |
| | OrnGGPSSGAPPPS-NH₂ |
| P442 | C6- |
| | HSDAVFTEQY(OMe)TOrnLRAibQVAAAibOrnYLQSIOrnCOrnGGPSSGAP |
| | PPS-NH₂ |
| P444 | C6- |
| | HSDAVFTEQY(OMe)TOrnLRAibQVAAAibOrnYLQSIOrnK(CO(CH₂)₂SH) |
| | OrnGGPSSGAPPPS-NH₂ |
| P446 | C6- |
| | HSDAVFTEQY(OMe)TOrnLRAibQVAAAibOrnYLQCIOrnOrnGGPSSGAPP |
| | PC-NH₂ |
| P448 | C6- |
| | HSDAVFTECY(OMe)TOrnLRAibQVAAAibOrnYLQSIOrnOrnGGPSSGAPP |
| | PS-NH₂ |
| P455 | C6- |
| | HSDAVFTEQY(OMe)TOrnCRAibQVAAAibOrnYLQSIOrnOrnGGPSSGAPP |
| | PS-NH₂ |
| P457 | C6- |
| | HSDAVFTEQY(OMe)TOrnK(CO(CH₂)₂SH)RAibQVAAAibOrnYLQSIOrn |
| | OrnGGPSSGAPPPS-NH₂ |

7. A VPAC2 receptor peptide agonist comprising a sequence of the formula:
Formula 4
(SEQ ID NO: 4)
Xaa₁-Xaa₂-Xaa₃-Xaa₄-Xaa₅-Xaa₆-Thr-Xaa₈-Xaa₉-Xaa₁₀-
Thr-Xaa₁₂-Xaa₁₃-Xaa₁₄-Xaa₁₅-Xaa₁₆-Xaa₁₇-Xaa₁₈-Abu-
Xaa₂₀-Xaa₂₁-Xaa₂₂-Xaa₂₃-Xaa₂₄-Xaa₂₅-Xaa₂₆-Xaa₂₇-
Xaa₂₈-Xaa₂₉-Xaa₃₀-Xaa₃₁-Xaa₃₂-Xaa₃₃-Xaa₃₄-Xaa₃₅-
Xaa₃₆-Xaa₃₇-Xaa₃₈-Xaa₃₉-Xaa₄₀
wherein:
Xaa₁ is: His, dH, or is absent;
Xaa₂ is: dA, Ser, Val, Gly, Thr, Leu, dS, Pro, or Aib;
Xaa₃ is: Asp or Glu;
Xaa₄ is: Ala, Ile, Tyr, Phe, Val, Thr, Leu, Trp, Gly, dA, Aib, or NMeA;
Xaa₅ is: Val, Leu, Phe, Ile, Thr, Trp, Tyr, dV, Aib, or NMeV;
Xaa₆ is: Phe, Ile, Leu, Thr, Val, Trp, or Tyr;
Xaa₈ is: Asp, Glu, Ala, Lys, Leu, Arg, or Tyr;
Xaa₉ is: Asn, Gln, Asp, Glu, Ser, Cys, Lys, or K(CO(CH₂)₂SH);
Xaa₁₀ is: Tyr, Trp, Tyr(OMe), Ser, Cys, or Lys;
Xaa₁₂ is: Arg, Lys, Glu, hR, Orn, Lys (isopropyl), Aib, Cit, Ala, Leu, Gln, Phe, Ser, or Cys;
Xaa₁₃ is: Leu, Phe, Glu, Ala, Aib, Ser, Cys, Lys, or K(CO(CH₂)₂SH);
Xaa₁₄ is: Arg, Leu, Lys, Ala, hR, Orn, Lys (isopropyl), Phe, Gln, Aib, Cit, Ser, or Cys;
Xaa₁₅ is: Lys, Ala, Arg, Glu, Leu, hR, Orn, Lys (isopropyl), Phe, Gln, Aib, K(Ac), Cit, Ser, Cys, K(W), or K(CO(CH₂)₂SH);
Xaa₁₆ is: Gln, Lys, Glu, Ala, hR, Orn, Lys (isopropyl), Cit, Ser, Cys, K(CO(CH₂)₂SH), or K(W);
Xaa₁₇ is: Val, Ala, Leu, Ile, Met, Nle, Lys, Aib, Ser, Cys, K(CO(CH₂)₂SH), or K(W);
Xaa₁₈ is: Ala, Ser, Cys, Lys, K(CO(CH₂)₂SH), K(W), Abu or Nle;
Xaa₂₀ is: Lys, Gln, hR, Arg, Ser, His, Orn, Lys (isopropyl), Ala, Aib, Trp, Thr, Leu, Ile, Phe, Tyr, Val, K(Ac), Cit, Cys, K(CO(CH₂)₂SH), or K(W);
Xaa₂₁ is: Lys, His, Arg, Ala, Phe, Aib, Leu, Gln, Orn, hR, K(Ac), Cit, Ser, Cys, Val, Tyr, Ile, Thr, Trp, K(W), or K(CO(CH₂)₂SH);
Xaa₂₂ is: Tyr, Trp, Phe, Thr, Leu, Ile, Val, Tyr(OMe), Ala, Aib, Ser, Cys, Lys, K(W), or K(CO(CH₂)₂SH);
Xaa₂₃ is: Leu, Phe, Ile, Ala, Trp, Thr, Val, Aib, Ser, Cys, Lys, K(W), or K(CO(CH₂)₂SH);
Xaa₂₄ is: Gln, Glu, Asn, Ser, Cys, Lys, K(CO(CH₂)₂SH), or K(W);
Xaa₂₅ is: Ser, Asp, Phe, Ile, Leu, Thr, Val, Trp, Gln, Asn, Tyr, Aib, Glu, Cys, Lys, K(CO(CH₂)₂SH), or K(W);
Xaa₂₆ is: Ile, Leu, Thr, Val, Trp, Tyr, Phe, Aib, Ser, Cys, Lys, K(CO(CH₂)₂SH), or K(W);
Xaa₂₇ is: Lys, hR, Arg, Gln, Ala, Asp, Glu, Phe, Gly, His, Ile, Met, Asn, Pro, Ser, Thr, Val, Trp, Tyr, Lys (isopropyl), Cys, Leu, Orn, dK, K(W), or K(CO(CH₂)₂SH);
Xaa₂₈ is: Asn, Asp, Gln, Lys, Arg, Aib, Orn, hR, Cit, Pro, dK, Ser, Cys, K(CO(CH₂)₂SH), or K(W);
Xaa₂₉ is: Lys, Ser, Arg, Asn, hR, Ala, Asp, Glu, Phe, Gly, His, Ile, Leu, Met, Pro, Gln, Thr, Val, Trp, Tyr, Cys, Orn, Cit, Aib, K(W), K(CO(CH₂)₂SH), or is absent;
Xaa₃₀ is: Arg, Lys, Ile, Ala, Asp, Glu, Phe, Gly, His, Leu, Met, Asn, Pro, Gln, Ser, Thr, Val, Trp, Tyr, Cys, hR, Cit, Aib, Orn, K(W), K(CO(CH₂)₂SH), or is absent;
Xaa₃₁ is: Tyr, His, Phe, Thr, Cys, Ser, Lys, Gln, K(W), K(CO(CH₂)₂SH), or is absent;
Xaa₃₂ is: Ser, Cys, Lys, or is absent;
Xaa₃₃ is: Trp or is absent;
Xaa₃₄ is: Cys or is absent;
Xaa₃₅ is: Glu or is absent;
Xaa₃₆ is: Pro or is absent;
Xaa₃₇ is: Gly or is absent;
Xaa₃₈ is: Trp or is absent;
Xaa₃₉ is: Cys or is absent; and
Xaa₄₀ is: Arg or is absent
wherein if Xaa₂₉, Xaa₃₀, Xaa₃₁, Xaa₃₂, Xaa₃₃, Xaa₃₄, Xaa₃₅, Xaa₃₆, Xaa₃₇, Xaa₃₈, or Xaa₃₉ is absent, the next amino acid present downstream is the next amino acid in said peptide agonist sequence,
and a C-terminal extension, wherein the N-terminus of said C-terminal extension is linked to the C-terminus of said peptide of Formula 4 and wherein said C-terminal extension comprises an amino acid sequence of the formula:
Formula 3
(SEQ ID NO: 3)
Xaa₁-Xaa₂-Xaa₃-Xaa₄-Xaa₅-Xaa₆-Xaa₇-Xaa₈-Xaa₉-
Xaa₁₀-Xaa₁₁-Xaa₁₂
wherein:
Xaa₁ is: Gly, Cys, or absent;
Xaa₂ is: Gly, Arg, or absent;
Xaa₃ is: Pro, Thr, or absent;
Xaa₄ is: Ser, or absent;
Xaa₅ is: Ser, or absent;
Xaa₆ is: Gly, or absent;
Xaa₇ is: Ala, or absent;
Xaa₈ is: Pro, or absent;
Xaa₉ is: Pro, or absent;
Xaa₁₀ is: Pro, or absent;
Xaa₁₁ is: Ser, Cys, or absent; and
Xaa₁₂ is: Cys, or absent;
wherein at least five of Xaa₁ to Xaa₁₂ of said C-terminal extension are present and wherein if Xaa₁, Xaa₂, Xaa₃, Xaa₄, Xaa₅, Xaa₆, Xaa₇, Xaa₈, Xaa₉, Xaa₁₀, or Xaa₁₁ is absent, the next amino acid present downstream is the next amino acid in said C-terminal extension and wherein the C-terminal amino acid may be amidated.

8. A pharmaceutical composition, comprising a VPAC2 receptor peptide agonist according to any one of claims 1 to 7 and one or more pharmaceutically acceptable diluents, carriers, or excipients.

9-11. (canceled)

12. A method of treating non-insulin dependent or insulin dependent diabetes in a patient in need thereof, comprising administering to said patient an effective amount of a VPAC2 receptor peptide agonist according to any one of claims 1 to 7.

13-17. (canceled)


SEQUENCE LISTING (256 sequences)

SEQ ID NO: 1 - protein, 13 aa, Artificial sequence
  Synthetic sequence C-terminal extension of the VPAC2 receptor peptide agonist
XXXXXXXXXXXXX

SEQ ID NO: 2 - protein, 13 aa, Artificial sequence
  Synthetic sequence C-terminal extension of the VPAC2 receptor peptide agonist
XXXXXXXXXXXXX

SEQ ID NO: 3 - protein, 12 aa, Artificial sequence
  Synthetic sequence C-terminal extension of the VPAC2 receptor peptide agonist
XXXXXXXXXXXX

SEQ ID NO: 4 - protein, 40 aa, Artificial sequence
  Synthetic sequence VPAC2 receptor peptide agonist
XXXXXXTXXXTXXXXXXXXXXXXXXXXXXXXXXXXXXXXX

SEQ ID NO: 5 - protein, 11 aa, Artificial sequence
  Synthetic sequence VPAC2 receptor peptide agonist
GGPSSGAPPPS

SEQ ID NO: 6 - protein, 11 aa, Artificial sequence
  Synthetic sequence VPAC2 receptor peptide agonist
GGPSSGAPPPS

SEQ ID NO: 7 - protein, 11 aa, Artificial sequence
  Synthetic sequence VPAC2 receptor peptide agonist
GGPSSGAPPPC

SEQ ID NO: 8 - protein, 11 aa, Artificial sequence
  Synthetic sequence VPAC2 receptor peptide agonist
GGPSSGAPPPC

SEQ ID NO: 9 - protein, 11 aa, Artificial sequence
  Synthetic sequence VPAC2 receptor peptide agonist
GRPSSGAPPPS

SEQ ID NO: 10 - protein, 11 aa, Artificial sequence
  Synthetic sequence VPAC2 receptor peptide agonist
GRPSSGAPPPS

SEQ ID NO: 11 - protein, 12 aa, Artificial sequence
  Synthetic sequence VPAC2 receptor peptide agonist
GGPSSGAPPPCC

SEQ ID NO: 12 - protein, 12 aa, Artificial sequence
  Synthetic sequence VPAC2 receptor peptide agonist
GGPSSGAPPPCC

SEQ ID NO: 13 - protein, 10 aa, Artificial sequence
  Synthetic sequence C-terminal extension of the VPAC2 receptor peptide agonist
XXXXXXXXXX

SEQ ID NO: 14 - protein, 28 aa, Homo sapiens
HSDAVFTDNYTRLRKQMAVKKYLNSILN

SEQ ID NO: 15 - protein, 27 aa, Homo sapiens
  MOD_RES (27)..(27): AMIDATION
HSDGIFTDSYSRYRKQMAVKKYLAAVL

SEQ ID NO: 16 - protein, 37 aa, Homo sapiens
  MOD_RES (37)..(37): AMIDATION
HSDGIFTDSYSRYRKQMAVKKYLAAVLGKRYQRVKNK

SEQ ID NO: 17 - protein, 29 aa, Artificial sequence
  Synthetic sequence VPAC2 receptor peptide agonist
HSDAVFTEQYTRXRXQLAAXXYLQSIKXX

SEQ ID NO: 18 - protein, 28 aa, Artificial sequence
  Synthetic sequence VPAC2 receptor peptide agonist
HSDAVFTEXYTXLRXQVAAXXYLQSIXX

SEQ ID NO: 19 - protein, 29 aa, Artificial sequence
  Synthetic sequence VPAC2 receptor peptide agonist
HSDAVFTEQYTXLRXQVAAXXYLQSIXXX

SEQ ID NO: 20 - protein, 28 aa, Artificial sequence
  Synthetic sequence VPAC2 receptor peptide agonist
HSDAVFTEQYTXLRXQVAAXXYLQSIXX

SEQ ID NO: 21 - protein, 28 aa, Artificial sequence
  Synthetic sequence VPAC2 receptor peptide agonist
HSDAVFTEQYTXLRXQVAAXXYLQSIXX

SEQ ID NO: 22 - protein, 28 aa, Artificial sequence
  Synthetic sequence VPAC2 receptor peptide agonist
HSDAVFTEQYTXLRXQVCAXXYLQSIXX

SEQ ID NO: 23 - protein, 28 aa, Artificial sequence
  Synthetic sequence VPAC2 receptor peptide agonist
HSDAVFTEQYTXLRCQVAAXXYLQSIXX

SEQ ID NO: 24 - protein, 28 aa, Artificial sequence
  Synthetic sequence VPAC2 receptor peptide agonist
HSDAVFTEQYTXLRXQLAAXXYLQSIXX

SEQ ID NO: 25 - protein, 28 aa, Artificial sequence
  Synthetic sequence VPAC2 receptor peptide agonist
HSDAVFTEQYTXLRXQVAAXXYXQSIXX

SEQ ID NO: 26 - protein, 28 aa, Artificial sequence
  Synthetic sequence VPAC2 receptor peptide agonist
HSDAVFTEQYTXLRXQVAAXXYLQXIXX

SEQ ID NO: 27 - protein, 28 aa, Artificial sequence
  Synthetic sequence VPAC2 receptor peptide agonist
HSDAVFTEQYTXLRXQVAXXXYLQXIXX

SEQ ID NO: 28 - protein, 28 aa, Artificial sequence
  Synthetic sequence VPAC2 receptor peptide agonist
HSDAVFTEQYTXLRXQLAAXXYLQXIXX

SEQ ID NO: 29 - protein, 28 aa, Artificial sequence
  Synthetic sequence VPAC2 receptor peptide agonist
HSDAVFTEQYTXLRXQLAAXXYXQXIXX

SEQ ID NO: 30 - protein, 28 aa, Artificial sequence
  Synthetic sequence VPAC2 receptor peptide agonist
HSDAVFTEQYTXLRXQLAXXXYXQSIXX

SEQ ID NO: 31 - protein, 28 aa, Artificial sequence
  Synthetic sequence VPAC2 receptor peptide agonist
HSDAVFTEQYTXLRXQLAXXXYLQSIXX

SEQ ID NO: 32 - protein, 28 aa, Artificial sequence
  Synthetic sequence VPAC2 receptor peptide agonist
HSDAVFTEQYTXLRXQLAXXXYXQXIXX

SEQ ID NO: 33 - protein, 28 aa, Artificial sequence
  Synthetic sequence VPAC2 receptor peptide agonist
HSDAVFTEQYTXLRXQLAAXXYXQSIXX

SEQ ID NO: 34 - protein, 28 aa, Artificial sequence
  Synthetic sequence VPAC2 receptor peptide agonist
HSDAVFTEQYTXLRXQVAAXXYLQSIXX

SEQ ID NO: 35 - protein, 28 aa, Artificial sequence
  Synthetic sequence VPAC2 receptor peptide agonist
HSDAVFTEQYTXLRXQVAAXXYLXSIXX

SEQ ID NO: 36 - protein, 28 aa, Artificial sequence
  Synthetic sequence VPAC2 receptor peptide agonist
HSDAVFTEQYTXLRXQXAAXXYLQSIXX

SEQ ID NO: 37 - protein, 28 aa, Artificial sequence
  Synthetic sequence VPAC2 receptor peptide agonist
HSDAVFTEQYTXLRXQVAAXXYLQSIXX

SEQ ID NO: 38 - protein, 28 aa, Artificial sequence
  Synthetic sequence VPAC2 receptor peptide agonist
HSDAVFTEQYTXLRXQVAAXXYLQSIXX

SEQ ID NO: 39 - protein, 28 aa, Artificial sequence
  Synthetic sequence VPAC2 receptor peptide agonist
HSDAVFTEQYTXLRXQVAAXCYLQSIXX

SEQ ID NO: 40 - protein, 28 aa, Artificial sequence
  Synthetic sequence VPAC2 receptor peptide agonist
HSDAVFTEQYTXLRXQLAXXXYLQXIXX

SEQ ID NO: 41 - protein, 28 aa, Artificial sequence
  Synthetic sequence VPAC2 receptor peptide agonist
HSDAVFTEQYTXLRXQVAAXXYLQSXXX

SEQ ID NO: 42 - protein, 29 aa, Artificial sequence
  Synthetic sequence VPAC2 receptor peptide agonist
HSDAVFTEQYTXLRXQLAXXXYLQXIXCX

SEQ ID NO: 43 - protein, 28 aa, Artificial sequence
  Synthetic sequence VPAC2 receptor peptide agonist
HSDAVFTEQYTXLRXQLAXXXYLQXCXX

SEQ ID NO: 44 - protein, 28 aa, Artificial sequence
  Synthetic sequence VPAC2 receptor peptide agonist
HSDAVFTEQYTXLRXQCAXXXYLQXIXX

SEQ ID NO: 45 - protein, 28 aa, Artificial sequence
  Synthetic sequence VPAC2 receptor peptide agonist
HSDAVFTEQYTXLRCQLAXXXYLQXIXX

SEQ ID NO: 46 - protein, 39 aa, Artificial sequence
  Synthetic sequence VPAC2 receptor peptide agonist
HSDAVFTEQYTXLRXQVAAXXYLQSIXXGGPSSGAPPPS

SEQ ID NO: 47 - protein, 40 aa, Artificial sequence
  Synthetic sequence VPAC2 receptor peptide agonist
HSDAVFTEQYTRXRXQLAAXXYLQSIKXXGGPSSGAPPPC

SEQ ID NO: 48 - protein, 39 aa, Artificial sequence
  Synthetic sequence VPAC2 receptor peptide agonist
HSDAVFTEXYTXLRXQVAAXXYLQSIXXGGPSSGAPPPS

SEQ ID NO: 49 - protein, 40 aa, Artificial sequence
  Synthetic sequence VPAC2 receptor peptide agonist
HSDAVFTEQYTXLRXQVAAXXYLQSIXXXGGPSSGAPPPS

SEQ ID NO: 50 - protein, 39 aa, Artificial sequence
  Synthetic sequence VPAC2 receptor peptide agonist
HSDAVFTEQYTXLRXQVAAXCYLQSIXXGGPSSGAPPPS

SEQ ID NO: 51 - protein, 39 aa, Artificial sequence; identical to SEQ ID NO: 46
  Synthetic sequence VPAC2 receptor peptide agonist

SEQ ID NO: 52 - protein, 39 aa, Artificial sequence; identical to SEQ ID NO: 46
  Synthetic sequence VPAC2 receptor peptide agonist

SEQ ID NO: 53 - protein, 39 aa, Artificial sequence
  Synthetic sequence VPAC2 receptor peptide agonist
HSDAVFTEQYTXLRXQVCAXXYLQSIXXGGPSSGAPPPS

SEQ ID NO: 54 - protein, 39 aa, Artificial sequence
  Synthetic sequence VPAC2 receptor peptide agonist
HSDAVFTEQYTXLRCQVAAXXYLQSIXXGGPSSGAPPPS

SEQ ID NO: 55 - protein, 39 aa, Artificial sequence
  Synthetic sequence VPAC2 receptor peptide agonist
HSDAVFTEQYTXLRXQLAAXXYLQSIXXGGPSSGAPPPS

SEQ ID NO: 56 - protein, 39 aa, Artificial sequence
  Synthetic sequence VPAC2 receptor peptide agonist
HSDAVFTEQYTXLRXQVAAXXYXQSIXXGGPSSGAPPPC

SEQ ID NO: 57 - protein, 39 aa, Artificial sequence
  Synthetic sequence VPAC2 receptor peptide agonist
HSDAVFTEQYTXLRXQVAAXXYLQXIXXGGPSSGAPPPC

SEQ ID NO: 58 - protein, 39 aa, Artificial sequence
  Synthetic sequence VPAC2 receptor peptide agonist
HSDAVFTEQYTXLRXQVAXXXYLQXIXXGGPSSGAPPPC

SEQ ID NO: 59 - protein, 40 aa, Artificial sequence
  Synthetic sequence VPAC2 receptor peptide agonist
HSDAVFTEQYTXLRXQLAAXXYLQXIXXGGPSSGAPPPCC

SEQ ID NO: 60 - protein, 40 aa, Artificial sequence
  Synthetic sequence VPAC2 receptor peptide agonist
HSDAVFTEQYTXLRXQLAXXXYLQXIXXGGPSSGAPPPCC

SEQ ID NO: 61 - protein, 40 aa, Artificial sequence
  Synthetic sequence VPAC2 receptor peptide agonist
HSDAVFTEQYTXLRXQLAAXXYXQXIXXGGPSSGAPPPCC

SEQ ID NO: 62 - protein, 40 aa, Artificial sequence
  Synthetic sequence VPAC2 receptor peptide agonist
HSDAVFTEQYTXLRXQLAXXXYXQSIXXGGPSSGAPPPCC

SEQ ID NO: 63 - protein, 40 aa, Artificial sequence
  Synthetic sequence VPAC2 receptor peptide agonist
HSDAVFTEQYTXLRXQLAXXXYLQSIXXGGPSSGAPPPCC

SEQ ID NO: 64 - protein, 40 aa, Artificial sequence
  Synthetic sequence VPAC2 receptor peptide agonist
HSDAVFTEQYTXLRXQLAXXXYXQXIXXGGPSSGAPPPCC

SEQ ID NO: 65 - protein, 40 aa, Artificial sequence
  Synthetic sequence VPAC2 receptor peptide agonist
HSDAVFTEQYTXLRXQLAAXXYXQSIXXGGPSSGAPPPCC

SEQ ID NO: 66 - protein, 39 aa, Artificial sequence; identical to SEQ ID NO: 46
  Synthetic sequence VPAC2 receptor peptide agonist

SEQ ID NO: 67 - protein, 39 aa, Artificial sequence
  Synthetic sequence VPAC2 receptor peptide agonist
HSDAVFTEQYTXLRXQVAAXXYLXSIXXGGPSSGAPPPS

SEQ ID NO: 68 - protein, 39 aa, Artificial sequence
  Synthetic sequence VPAC2 receptor peptide agonist
HSDAVFTEQYTXLRXQXAAXXYLQSIXXGGPSSGAPPPS

SEQ ID NO: 69 - protein, 39 aa, Artificial sequence
  Synthetic sequence VPAC2 receptor peptide agonist
HSDAVFTEQYTXLRXQVAAXXYLQSXXXGGPSSGAPPPS

SEQ ID NO: 70 - protein, 40 aa, Artificial sequence
  Synthetic sequence VPAC2 receptor peptide agonist
HSDAVFTEQYTXLRXQLAXXXYLQXIXCXGGPSSGAPPPS

SEQ ID NO: 71 - protein, 39 aa, Artificial sequence
  Synthetic sequence VPAC2 receptor peptide agonist
HSDAVFTEQYTXLRXQLAXXXYLQXCXXGGPSSGAPPPS

SEQ ID NO: 72 - protein, 39 aa, Artificial sequence
  Synthetic sequence VPAC2 receptor peptide agonist
HSDAVFTEQYTXLRXQCAXXXYLQXIXXGGPSSGAPPPS

SEQ ID NO: 73 - protein, 39 aa, Artificial sequence
  Synthetic sequence VPAC2 receptor peptide agonist
HSDAVFTEQYTXLRCQLAXXXYLQXIXXGGPSSGAPPPS

SEQ ID NO: 74 - protein, 39 aa, Artificial sequence; identical to SEQ ID NO: 46
  Synthetic sequence VPAC2 receptor peptide agonist

SEQ ID NO: 75 - protein, 39 aa, Artificial sequence
  Synthetic sequence VPAC2 receptor peptide agonist
HSDAVFTEQYTXLRXQVAAXXYLXSIXXGGPSSGAPPPC

SEQ ID NO: 76 - protein, 39 aa, Artificial sequence
  Synthetic sequence VPAC2 receptor peptide agonist
HSDAVFTEQYTXLRXQVAXXXYLQSIXXGGPSSGAPPPC

SEQ ID NO: 77 - protein, 39 aa, Artificial sequence; identical to SEQ ID NO: 57
  Synthetic sequence VPAC2 receptor peptide agonist

SEQ ID NO: 78 - protein, 39 aa, Artificial sequence
  Synthetic sequence VPAC2 receptor peptide agonist
HSDAVFTEQYTXLRXQVAAXXYLQSXXXGGPSSGAPPPC

SEQ ID NO: 79 - protein, 40 aa, Artificial sequence
  Synthetic sequence VPAC2 receptor peptide agonist
HSDAVFTEQYTXLRXQVAAXXYLQSIXXXGGPSSGAPPPC

SEQ ID NO: 80 - protein, 39 aa, Artificial sequence
  Synthetic sequence VPAC2 receptor peptide agonist
HSDAVFTEQYTXLRXQVAAXXYLCSIXXGGPSSGAPPPC

SEQ ID NO: 81 - protein, 39 aa, Artificial sequence
  Synthetic sequence VPAC2 receptor peptide agonist
HSDAVFTEQYTXLRXQVAAXXYLQSCXXGGPSSGAPPPC

SEQ ID NO: 82 - protein, 40 aa, Artificial sequence
  Synthetic sequence VPAC2 receptor peptide agonist
HSDAVFTEQYTXLRXQVAAXXYLQSIXCXGGPSSGAPPPC

SEQ ID NO: 83 - protein, 7 aa, Artificial sequence
  Synthetic sequence VPAC2 receptor peptide agonist
SRTSPPP

SEQ ID NO: 84 - protein, 7 aa, Artificial sequence
  Synthetic sequence VPAC2 receptor peptide agonist
SRTSPPP

SEQ ID NO: 85 - protein, 10 aa, Artificial sequence
  Synthetic sequence VPAC2 receptor peptide agonist
SSTSPRPPSS

SEQ ID NO: 86 - protein, 10 aa, Artificial sequence
  Synthetic sequence VPAC2 receptor peptide agonist
SSTSPRPPSS

SEQ ID NO: 87 - protein, 8 aa, Artificial sequence
  Synthetic sequence VPAC2 receptor peptide agonist
SRTSPPPX

SEQ ID NO: 88 - protein, 8 aa, Artificial sequence
  Synthetic sequence VPAC2 receptor peptide agonist
SRTSPPPX

SEQ ID NO: 89 - protein, 8 aa, Artificial sequence
  Synthetic sequence VPAC2 receptor peptide agonist
SRTSPPPC

SEQ ID NO: 90 - protein, 8 aa, Artificial sequence
  Synthetic sequence VPAC2 receptor peptide agonist
SRTSPPPC

SEQ ID NO: 91 - protein, 9 aa, Artificial sequence
  Synthetic sequence N-terminal modification to VPAC2 receptor peptide agonist
SWCEPGWCR

SEQ ID NO: 92 - protein, 28 aa, Artificial sequence
  Synthetic sequence VPAC2 receptor peptide agonist
HSDAVFTEQYTXLRXQVXAXXYLQSIXX

SEQ ID NO: 93 - protein, 29 aa, Artificial sequence
  Synthetic sequence VPAC2 receptor peptide agonist
HSDAVFTEQYTXLRXQLAXXXYLQSIXCX

SEQ ID NO: 94 - protein, 28 aa, Artificial sequence
  Synthetic sequence VPAC2 receptor peptide agonist
HSDAVFTEQYTXLRXQLAXXXYLQSCXX

SEQ ID NO: 95 - protein, 29 aa, Artificial sequence
  Synthetic sequence VPAC2 receptor peptide agonist
HSDAVFTEQYTXLRXQLAXXXYLQXIXXX

SEQ ID NO: 96 - protein, 29 aa, Artificial sequence
  Synthetic sequence VPAC2 receptor peptide agonist
HSDAVFTEQYTXLRXQLAXXXYLQSIXXX

SEQ ID NO: 97 - protein, 28 aa, Artificial sequence
  Synthetic sequence VPAC2 receptor peptide agonist
HSDAVFTEQYTXLRXQLAXXXYLQXIXX

SEQ ID NO: 98 - protein, 29 aa, Artificial sequence
  Synthetic sequence VPAC2 receptor peptide agonist
HSDAVFTEQYTXLRXQLAAXXYLQSIXXC

SEQ ID NO: 99 - protein, 29 aa, Artificial sequence
  Synthetic sequence VPAC2 receptor peptide agonist
HSDAVFTEQYTXLRXQVAAXXYLQSIXXC

SEQ ID NO: 100 - protein, 29 aa, Artificial sequence
  Synthetic sequence VPAC2 receptor peptide agonist
HSDAVFTEQYTXLRXQLAXXXYLQSIXXC

SEQ ID NO: 101 - protein, 28 aa, Artificial sequence
  Synthetic sequence VPAC2 receptor peptide agonist
HSDAVFTEQYTXLRXQLAXXXYLQXIXX

SEQ ID NO: 102 - protein, 29 aa, Artificial sequence
  Synthetic sequence VPAC2 receptor peptide agonist
HSDAVFTEQYTXLRXQLAXXXYLQXIXCX

SEQ ID NO: 103 - protein, 28 aa, Artificial sequence
  Synthetic sequence VPAC2 receptor peptide agonist
HSDAVFTEQYTXLRXQCAXXXYLQXIXX

SEQ ID NO: 104 - protein, 29 aa, Artificial sequence
  Synthetic sequence VPAC2 receptor peptide agonist
HSDAVFTEQYTXLRXQLAXXXYLQXIXXC

SEQ ID NO: 105 - protein, 28 aa, Artificial sequence
  Synthetic sequence VPAC2 receptor peptide agonist
HSDAVFTEQYTXLRXQLAXXXYLQSIXX

SEQ ID NO: 106 - protein, 28 aa, Artificial sequence
  Synthetic sequence VPAC2 receptor peptide agonist
HSDAVFTEQYTXLRXQCAXXXYLQSIXX

SEQ ID NO: 107 - protein, 29 aa, Artificial sequence
  Synthetic sequence VPAC2 receptor peptide agonist
HSDAVFTEQYTXLRXQLAXXXYLQSIXCX

SEQ ID NO: 108 - protein, 28 aa, Artificial sequence
  Synthetic sequence VPAC2 receptor peptide agonist
HSDAVFTEQYTXLRXQLXAXXYLQSIXX

SEQ ID NO: 109 - protein, 28 aa, Artificial sequence
  Synthetic sequence VPAC2 receptor peptide agonist
HSDAVFTEQYTXLRXQXAXXXYLQXIXX

SEQ ID NO: 110 - protein, 28 aa, Artificial sequence
  Synthetic sequence VPAC2 receptor peptide agonist
HSDAVFTEQYTXLRXQXAXXXYLQXIXX

SEQ ID NO: 111 - protein, 39 aa, Artificial sequence
  Synthetic sequence VPAC2 receptor peptide agonist
HSDAVFTEQYTXLRXQVXAXXYLQSIXXGGPSSGAPPPS

SEQ ID NO: 112 - protein, 40 aa, Artificial sequence
  Synthetic sequence VPAC2 receptor peptide agonist
HSDAVFTEQYTXLRXQLAXXXYLQSIXCXGGPSSGAPPPS

SEQ ID NO: 113 - protein, 39 aa, Artificial sequence
  Synthetic sequence VPAC2 receptor peptide agonist
HSDAVFTEQYTXLRXQLAXXXYLQSCXXGGPSSGAPPPS

SEQ ID NO: 114 - protein, 40 aa, Artificial sequence
  Synthetic sequence VPAC2 receptor peptide agonist
HSDAVFTEQYTXLRXQLAXXXYLQXIXXXGGPSSGAPPPC

SEQ ID NO: 115 - protein, 40 aa, Artificial sequence
  Synthetic sequence VPAC2 receptor peptide agonist
HSDAVFTEQYTXLRXQLAXXXYLQSIXXXGGPSSGAPPPC

SEQ ID NO: 116 - protein, 40 aa, Artificial sequence
  Synthetic sequence VPAC2 receptor peptide agonist
HSDAVFTEQYTXLRXQLAXXXYLQSIXCXGGPSSGAPPPC

SEQ ID NO: 117 - protein, 39 aa, Artificial sequence
  Synthetic sequence VPAC2 receptor peptide agonist
HSDAVFTEQYTXLRXQLAXXXYLQXIXXGGPSSGAPPPS

SEQ ID NO: 118 - protein, 40 aa, Artificial sequence
  Synthetic sequence VPAC2 receptor peptide agonist
HSDAVFTEQYTXLRXQLAAXXYLQSIXXCGGPSSGAPPPS

SEQ ID NO: 119 - protein, 40 aa, Artificial sequence
  Synthetic sequence VPAC2 receptor peptide agonist
HSDAVFTEQYTXLRXQVAAXXYLQSIXXCGGPSSGAPPPC

SEQ ID NO: 120 - protein, 40 aa, Artificial sequence
  Synthetic sequence VPAC2 receptor peptide agonist
HSDAVFTEQYTXLRXQLAXXXYLQXIXXCGGPSSGAPPPS

SEQ ID NO: 121 - protein, 40 aa, Artificial sequence
  Synthetic sequence VPAC2 receptor peptide agonist
HSDAVFTEQYTXLRXQLAXXXYLQSIXXCGGPSSGAPPPS

SEQ ID NO: 122 - protein, 40 aa, Artificial sequence
  Synthetic sequence VPAC2 receptor peptide agonist
HSDAVFTEQYTXLRXQLAXXXYLQSIXXCGGPSSGAPPPC

SEQ ID NO: 123 - protein, 40 aa, Artificial sequence; identical to SEQ ID NO: 60
  Synthetic sequence VPAC2 receptor peptide agonist

SEQ ID NO: 124 - protein, 40 aa, Artificial sequence; identical to SEQ ID NO: 70
  Synthetic sequence VPAC2 receptor peptide agonist

SEQ ID NO: 125 - protein, 39 aa, Artificial sequence; identical to SEQ ID NO: 72
  Synthetic sequence VPAC2 receptor peptide agonist

SEQ ID NO: 126 - protein, 40 aa, Artificial sequence
  Synthetic sequence VPAC2 receptor peptide agonist
HSDAVFTEQYTXLRXQLAXXXYLQXIXXCGGPSSGAPPPC

SEQ ID NO: 127 - protein, 40 aa, Artificial sequence; identical to SEQ ID NO: 63
  Synthetic sequence VPAC2 receptor peptide agonist

SEQ ID NO: 128 - protein, 40 aa, Artificial sequence; identical to SEQ ID NO: 112
  Synthetic sequence VPAC2 receptor peptide agonist

SEQ ID NO: 129 - protein, 39 aa, Artificial sequence
  Synthetic sequence VPAC2 receptor peptide agonist
HSDAVFTEQYTXLRXQCAXXXYLQSIXXGGPSSGAPPPS

SEQ ID NO: 130 - protein, 40 aa, Artificial sequence; identical to SEQ ID NO: 116
  Synthetic sequence VPAC2 receptor peptide agonist

SEQ ID NO: 131 - protein, 40 aa, Artificial sequence
  Synthetic sequence VPAC2 receptor peptide agonist
HSDAVFTEQYTXLRXQLXAXXYLQSIXXGGPSSGAPPPCC

SEQ ID NO: 132 - protein, 39 aa, Artificial sequence
  Synthetic sequence VPAC2 receptor peptide agonist
HSDAVFTEQYTXLRXQXAXXXYLQXIXXGGPSSGAPPPC

SEQ ID NO: 133 - protein, 39 aa, Artificial sequence
  Synthetic sequence VPAC2 receptor peptide agonist
HSDAVFTEQYTXLRXQXAXXXYLQXIXXGGPSSGAPPPS

SEQ ID NO: 134 - protein, 39 aa, Artificial sequence; identical to SEQ ID NO: 133
  Synthetic sequence VPAC2 receptor peptide agonist

SEQ ID NO: 135 - protein, 39 aa, Artificial sequence
  Synthetic sequence VPAC2 receptor peptide agonist
HSDAVFTEQYTXLRXQVAAXXYLQSIXXGGPSSGAPPPX

SEQ ID NO: 136 - protein, 39 aa, Artificial sequence; identical to SEQ ID NO: 46
  Synthetic sequence VPAC2 receptor peptide agonist

SEQ ID NO: 137 - protein, 40 aa, Artificial sequence
  Synthetic sequence VPAC2 receptor peptide agonist
HSDAVFTEQYTXLRXQLAXXXYLQXIXCXGGPSSGAPPPC

SEQ ID NO: 138 - protein, 39 aa, Artificial sequence
  Synthetic sequence VPAC2 receptor peptide agonist
HSDAVFTEQYTXLRXQCAAXXYLQSIXXGGPSSGAPPPC

SEQ ID NO: 139 - protein, 39 aa, Artificial sequence
  Synthetic sequence VPAC2 receptor peptide agonist
HSDAVFTEQYTXLRXQLAXXXYXQXIXXGGPSSGAPPPS

SEQ ID NO: 140 - protein, 39 aa, Artificial sequence
  Synthetic sequence VPAC2 receptor peptide agonist
HSDAVFTEQYTXLRXQLAXXXYXQXIXXGGPSSGAPPPX

SEQ ID NO: 141 - protein, 39 aa, Artificial sequence
  Synthetic sequence VPAC2 receptor peptide agonist
HSDAVFTEQYTXLRXQCAXXXYLQXIXXGGPSSGAPPPC

SEQ ID NO: 142 - protein, 43 aa, Artificial sequence
  Synthetic sequence VPAC2 receptor peptide agonist
XHSDAVFTDNYTRLRXQVAAXKYLQSIKNKRYGGPSSGAPPPS

SEQ ID NO: 143 - protein, 43 aa, Artificial sequence; identical to SEQ ID NO: 142
  Synthetic sequence VPAC2 receptor peptide agonist

SEQ ID NO: 144 - protein, 39 aa, Artificial sequence
  Synthetic sequence VPAC2 receptor peptide agonist
HSDAVFTDNYTRLRXQVAAXKYLQSIKQGRPSSGAPPPS

SEQ ID NO: 145 - protein, 31 aa, Artificial sequence
  Synthetic sequence VPAC2 receptor peptide agonist
HSDAVFTDNYTRLRKQVAAKKYLQSIKNKRQ

SEQ ID NO: 146 - protein, 29 aa, Artificial sequence
  Synthetic sequence VPAC2 receptor peptide agonist
HSDAVFTDNYTLLRXQVAAXKYLQSIXNX

SEQ ID NO: 147 - protein, 29 aa, Artificial sequence
  Synthetic sequence VPAC2 receptor peptide agonist
HSDAVFTDNYTQLRXQVAAXKYLQSIXNX

SEQ ID NO: 148 - protein, 29 aa, Artificial sequence
  Synthetic sequence VPAC2 receptor peptide agonist
HSDAVFTDNYTFLRXQVAAXKYLQSIXNX

SEQ ID NO: 149 - protein, 29 aa, Artificial sequence
  Synthetic sequence VPAC2 receptor peptide agonist
HSDAVFTDNYTXLRXQVAAXKYLQCIXNX

SEQ ID NO: 150 - protein, 29 aa, Artificial sequence
  Synthetic sequence VPAC2 receptor peptide agonist
HSDAVFTDNYTXLRXQVAACXYLQSIXNX

SEQ ID NO: 151 - protein, 29 aa, Artificial sequence
  Synthetic sequence VPAC2 receptor peptide agonist
HSDAVFTDNYTXLRXQVAAXKYLQSSXNX

SEQ ID NO: 152 - protein, 29 aa, Artificial sequence
  Synthetic sequence VPAC2 receptor peptide agonist
HSDAVFTDNYTXLRXQVAAXKYLSSIXNX

SEQ ID NO: 153 - protein, 29 aa, Artificial sequence
  MOD_RES (15, 20): Aib
HSDAVFTDNYTXLRXQVAAXKYSQSIXNX

SEQ ID NO: 154 - protein, 29 aa, Artificial sequence
  Synthetic sequence VPAC2 receptor peptide agonist
HSDAVFTDNYTXLRXQVAAXKSLQSIXNX

SEQ ID NO: 155 - protein, 29 aa, Artificial sequence
  Synthetic sequence VPAC2 receptor peptide agonist
HSDAVFTDNYTXLRXQVAAXSYLQSIXNX

SEQ ID NO: 156 - protein, 29 aa, Artificial sequence
  Synthetic sequence VPAC2 receptor peptide agonist
HSDAVFTDNYTXLRXQVSAXKYLQSIXNX

SEQ ID NO: 157 - protein, 29 aa, Artificial sequence
  Synthetic sequence VPAC2 receptor peptide agonist
HSDAVFTDNYTXLRXQSAAXKYLQSIXNX

SEQ ID NO: 158 - protein, 29 aa, Artificial sequence
  Synthetic sequence VPAC2 receptor peptide agonist
HSDAVFTDNYTXLRXSVAAXKYLQSIXNX

SEQ ID NO: 159 - protein, 29 aa, Artificial sequence
  Synthetic sequence VPAC2 receptor peptide agonist
HSDAVFTDNYTXSRXQVAAXKYLQSIXNX

SEQ ID NO: 160 - protein, 29 aa, Artificial sequence
  Synthetic sequence VPAC2 receptor peptide agonist
HSDAVFTDSYTXLRXQVAAXKYLQSIXNX

SEQ ID NO: 161 - protein, 31 aa, Artificial sequence
  Synthetic sequence VPAC2 receptor peptide agonist
HSDAVFTDNYTXLRXQVAAXKYLQSIKNKRY

SEQ ID NO: 162 - protein, 29 aa, Artificial sequence
  Synthetic sequence VPAC2 receptor peptide agonist
HSDAVFTDNYTRLRXQVAAXKYLQSIKXX

SEQ ID NO: 163 - protein, 31 aa, Artificial sequence
  Synthetic sequence VPAC2 receptor peptide agonist
HSDAVFTDNYTRLRXQVAAXKYLQSIKNKRY

SEQ ID NO: 164 - protein, 29 aa, Artificial sequence
  Synthetic sequence VPAC2 receptor peptide agonist
HSEAVFTENYTXLRXQVAAXKYLQSIXNX

SEQ ID NO: 165 - protein, 29 aa, Artificial sequence
  Synthetic sequence VPAC2 receptor peptide agonist
HSDAVFTDQYTXLRXQVAAXKYLQSIXQX

SEQ ID NO: 166 - protein, 29 aa, Artificial sequence
  Synthetic sequence VPAC2 receptor peptide agonist
HSDAVFTDNYTRLLAKLALQKYLQSIXNX

SEQ ID NO: 167 - protein, 29 aa, Artificial sequence
  Synthetic sequence VPAC2 receptor peptide agonist
HSDAVFTDNYTXLLAKLALQKYLQSIXNX

SEQ ID NO: 168 - protein, 28 aa, Artificial sequence
  Synthetic sequence VPAC2 receptor peptide agonist
HSEAVFTEQYTXLRXQVAAXXYLQSIXX

SEQ ID NO: 169 - protein, 29 aa, Artificial sequence
  Synthetic sequence VPAC2 receptor peptide agonist
HSDAVFTDNYTXLRXQVASXKYLQSIXNX

SEQ ID NO: 170 - protein, 28 aa, Artificial sequence
  Synthetic sequence VPAC2 receptor peptide agonist
HSEAVFTEQYTXLRXQLAAXXYLQSIXX

SEQ ID NO: 171 - protein, 28 aa, Artificial sequence
  Synthetic sequence VPAC2 receptor peptide agonist
HSDAVFTDQYTXLRXQLAAXXYLQSIXX

SEQ ID NO: 172 - protein, 28 aa, Artificial sequence
  Synthetic sequence VPAC2 receptor peptide agonist
HSDAVFTDQYTXLRXQLAAXXYLQSIXX

SEQ ID NO: 173 - protein, 28 aa, Artificial sequence
  Synthetic sequence VPAC2 receptor peptide agonist
HSDAVFTDQYTXLRXQVAAXXYLQSIXX

SEQ ID NO: 174 - protein, 28 aa, Artificial sequence
  Synthetic sequence VPAC2 receptor peptide agonist
HSDAVFTDNYTXLRXQVAAXXYLQSIXX

SEQ ID NO: 175 - protein, 28 aa, Artificial sequence
  Synthetic sequence VPAC2 receptor peptide agonist
HSDAVFTEQYTXLRXQVAAXXYLQSIXX

SEQ ID NO: 176 - protein, 29 aa, Artificial sequence
  Synthetic sequence VPAC2 receptor peptide agonist
HSDAVFTDNYTRXRXQVAAXKYLQSIKXK

SEQ ID NO: 177 - protein, 29 aa, Artificial sequence
  Synthetic sequence VPAC2 receptor peptide agonist
HSDAVFTDQYTRXRXQVAAXKYLQSIKXK

SEQ ID NO: 178 - protein, 29 aa, Artificial sequence
  Synthetic sequence VPAC2 receptor peptide agonist
HSDAVFTDQYTRXRXQLAAXKYLQSIKXK

SEQ ID NO: 179 - protein, 29 aa, Artificial sequence
  Synthetic sequence VPAC2 receptor peptide agonist
HSDAVFTDQYTRXRXQLAAXKYLQSIKXK

SEQ ID NO: 180 - protein, 29 aa, Artificial sequence
  Synthetic sequence VPAC2 receptor peptide agonist
HSEAVFTEQYTRXRXQLAAXKYLQSIKXK

SEQ ID NO: 181 - protein, 28 aa, Artificial sequence
  Synthetic sequence VPAC2 receptor peptide agonist
HSDAVFTEQYTXLRXQVAAXXYLCSIXX

SEQ ID NO: 182 - protein, 28 aa, Artificial sequence
  Synthetic sequence VPAC2 receptor peptide agonist
HSDAVFTEQYTXLRXQVAAXXYLXSIXX

SEQ ID NO: 183 - protein, 28 aa, Artificial sequence
  Synthetic sequence VPAC2 receptor peptide agonist
HSDAVFTEQYTXLRXQVACXXYLQSIXX

SEQ ID NO: 184 - protein, 28 aa, Artificial sequence
  Synthetic sequence VPAC2 receptor peptide agonist
HSDAVFTEQYTXLRXQVAXXXYLQSIXX

SEQ ID NO: 185 - protein, 28 aa, Artificial sequence
  Synthetic sequence VPAC2 receptor peptide agonist
HSDAVFTEQYTXLRXCVAAXXYLQSIXX

SEQ ID NO: 186 - protein, 29 aa, Artificial sequence
  Synthetic sequence VPAC2 receptor peptide agonist
HSDAVFTDNYTXLRXQVAAXKYLQSIXNX

SEQ ID NO: 187 - protein, 28 aa, Artificial sequence
  Synthetic sequence VPAC2 receptor peptide agonist
HSDAVFTEQYTXLRXQLAAXXYLQSIXX

SEQ ID NO: 188 - protein, 28 aa, Artificial sequence
  Synthetic sequence VPAC2 receptor peptide agonist
HSDAVFTEQYTXLRXQVAAXXYLQSCXX

SEQ ID NO: 189 - protein, 28 aa, Artificial sequence
  Synthetic sequence VPAC2 receptor peptide agonist
HSDAVFTEQYTXLRXQCAAXXYLQSIXX

SEQ ID NO: 190 - protein, 29 aa, Artificial sequence
  Synthetic sequence VPAC2 receptor peptide agonist
HSDAVFTEQYTXLRXQVAAXXYLQSIXCX

SEQ ID NO: 191 - protein, 28 aa, Artificial sequence
  Synthetic sequence VPAC2 receptor peptide agonist
HSDAVFTEQYTXLRXQVAAXXYLQCIXX

SEQ ID NO: 192 - protein, 28 aa, Artificial sequence
  Synthetic sequence VPAC2 receptor peptide agonist
HSDAVFTECYTXLRXQVAAXXYLQSIXX

SEQ ID NO: 193 - protein, 28 aa, Artificial sequence
  Synthetic sequence VPAC2 receptor peptide agonist
HSDAVFTEQYTXCRXQVAAXXYLQSIXX

SEQ ID NO: 194 - protein, 28 aa, Artificial sequence
  Synthetic sequence VPAC2 receptor peptide agonist
HSDAVFTEQYTXLRXQVAAXXYLQSXXX

SEQ ID NO: 195 - protein, 28 aa, Artificial sequence
  Synthetic sequence VPAC2 receptor peptide agonist
HSDAVFTEQYTXLRXQVAAXXYLQXIXX

SEQ ID NO: 196 - protein, 28 aa, Artificial sequence
  Synthetic sequence VPAC2 receptor peptide agonist
HSDAVFTEQYTXLRXQXAAXXYLQSIXX

SEQ ID NO: 197 - protein, 28 aa, Artificial sequence
  Synthetic sequence VPAC2 receptor peptide agonist
HSDAVFTEQYTXLRXXVAAXXYLQSIXX

SEQ ID NO: 198 - protein, 29 aa, Artificial sequence
  Synthetic sequence VPAC2 receptor peptide agonist
HSDAVFTEQYTXLRXQVAAXXYLQSIXXX

SEQ ID NO: 199 - protein, 28 aa, Artificial sequence
  Synthetic sequence VPAC2 receptor peptide agonist
HSDAVFTEQYTXXRXQVAAXXYLQSIXX

SEQ ID NO: 200 - protein, 42 aa, Artificial sequence
  Synthetic sequence VPAC2 receptor peptide agonist
HSDAVFTDNYTRLRKQVAAKKYLQSIKNKRQGGPSSGAPPPS

SEQ ID NO: 201 - protein, 42 aa, Artificial sequence
  Synthetic sequence VPAC2 receptor peptide agonist
HSDAVFTDNYTXLRXQVAAXKYLQSIKNKRYGGPSSGAPPPS

SEQ ID NO: 202 - protein, 40 aa, Artificial sequence
  Synthetic sequence VPAC2 receptor peptide agonist
HSDAVFTDNYTRLRXQVAAXKYLQSIKXXGGPSSGAPPPS

SEQ ID NO: 203 - protein, 42 aa, Artificial sequence
  Synthetic sequence VPAC2 receptor peptide agonist
HSDAVFTDNYTRLRXQVAAXKYLQSIKNKRYGGPSSGAPPPS

SEQ ID NO: 204 - protein, 40 aa, Artificial sequence
  Synthetic sequence VPAC2 receptor peptide agonist
HSEAVFTENYTXLRXQVAAXKYLQSIXNXGGPSSGAPPPS

SEQ ID NO: 205 - protein, 40 aa, Artificial sequence
  Synthetic sequence VPAC2 receptor peptide agonist
HSDAVFTDQYTXLRXQVAAXKYLQSIXQXGGPSSGAPPPS

SEQ ID NO: 206 - protein, 40 aa, Artificial sequence
  Synthetic sequence VPAC2 receptor peptide agonist
HSDAVFTDNYTLLRXQVAAXKYLQSIXNXGGPSSGAPPPS

SEQ ID NO: 207 - protein, 40 aa, Artificial sequence
  Synthetic sequence VPAC2 receptor peptide agonist
HSDAVFTDNYTQLRXQVAAXKYLQSIXNXGGPSSGAPPPS

SEQ ID NO: 208 - protein, 40 aa, Artificial sequence
  Synthetic sequence VPAC2 receptor peptide agonist
HSDAVFTDNYTFLRXQVAAXKYLQSIXNXGGPSSGAPPPS

SEQ ID NO: 209 - protein, 40 aa, Artificial sequence
  Synthetic sequence VPAC2 receptor peptide agonist
HSDAVFTDNYTRLLAKLALQKYLQSIXNXGGPSSGAPPPS

SEQ ID NO: 210 - protein, 40 aa, Artificial sequence
  Synthetic sequence VPAC2 receptor peptide agonist
HSDAVFTDNYTXLLAKLALQKYLQSIXNXGGPSSGAPPPS

SEQ ID NO: 211 - protein, 40 aa, Artificial sequence
  Synthetic sequence VPAC2 receptor peptide agonist
HSDAVFTDNYTXLRXQVAAXKYLQCIXNXGGPSSGAPPPS

SEQ ID NO: 212 - protein, 40 aa, Artificial sequence
  Synthetic sequence VPAC2 receptor peptide agonist
HSDAVFTDNYTXLRXQVAACXYLQSIXNXGGPSSGAPPPS

SEQ ID NO: 213 - protein, 39 aa, Artificial sequence
  Synthetic sequence VPAC2 receptor peptide agonist
HSEAVFTEQYTXLRXQVAAXXYLQSIXXGGPSSGAPPPC

SEQ ID NO: 214 - protein, 40 aa, Artificial sequence
  Synthetic sequence VPAC2 receptor peptide agonist
HSDAVFTDNYTXLRXQVAAXKYLQSSXNXGGPSSGAPPPS

SEQ ID NO: 215 - protein, 40 aa, Artificial sequence
  Synthetic sequence VPAC2 receptor peptide agonist
HSDAVFTDNYTXLRXQVAAXKYLSSIXNXGGPSSGAPPPS

SEQ ID NO: 216 - protein, 40 aa, Artificial sequence
  Synthetic sequence VPAC2 receptor peptide agonist
HSDAVFTDNYTXLRXQVAAXKYSQSIXNXGGPSSGAPPPS

SEQ ID NO: 217 - protein, 40 aa, Artificial sequence
  Synthetic sequence VPAC2 receptor peptide agonist
HSDAVFTDNYTXLRXQVAAXKSLQSIXNXGGPSSGAPPPS

SEQ ID NO: 218 - protein, 40 aa, Artificial sequence
  Synthetic sequence VPAC2 receptor peptide agonist
HSDAVFTDNYTXLRXQVAAXSYLQSIXNXGGPSSGAPPPS

SEQ ID NO: 219 - protein, 40 aa, Artificial sequence
  Synthetic sequence VPAC2 receptor peptide agonist
HSDAVFTDNYTXLRXQVASXKYLQSIXNXGGPSSGAPPPS

SEQ ID NO: 220 - protein, 40 aa, Artificial sequence
  Synthetic sequence VPAC2 receptor peptide agonist
HSDAVFTDNYTXLRXQVSAXKYLQSIXNXGGPSSGAPPPS

SEQ ID NO: 221 - protein, 40 aa, Artificial sequence
  Synthetic sequence VPAC2 receptor peptide agonist
HSDAVFTDNYTXLRXQSAAXKYLQSIXNXGGPSSGAPPPS

SEQ ID NO: 222 - protein, 40 aa, Artificial sequence
  Synthetic sequence VPAC2 receptor peptide agonist
HSDAVFTDNYTXLRXSVAAXKYLQSIXNXGGPSSGAPPPS

SEQ ID NO: 223 - protein, 40 aa, Artificial sequence
  Synthetic sequence VPAC2 receptor peptide agonist
HSDAVFTDNYTXSRXQVAAXKYLQSIXNXGGPSSGAPPPS

SEQ ID NO: 224 - protein, 40 aa, Artificial sequence
  Synthetic sequence VPAC2 receptor peptide agonist
HSDAVFTDSYTXLRXQVAAXKYLQSIXNXGGPSSGAPPPS

SEQ ID NO: 225 - protein, 39 aa, Artificial sequence
  Synthetic sequence VPAC2 receptor peptide agonist
HSEAVFTEQYTXLRXQLAAXXYLQSIXXGGPSSGAPPPS

SEQ ID NO: 226 - protein, 39 aa, Artificial sequence
  Synthetic sequence VPAC2 receptor peptide agonist
HSDAVFTDQYTXLRXQLAAXXYLQSIXXGGPSSGAPPPS

SEQ ID NO: 227 - protein, 39 aa, Artificial sequence; identical to SEQ ID NO: 226
  Synthetic sequence VPAC2 receptor peptide agonist

SEQ ID NO: 228 - protein, 39 aa, Artificial sequence
  Synthetic sequence VPAC2 receptor peptide agonist
HSDAVFTDQYTXLRXQVAAXXYLQSIXXGGPSSGAPPPS

SEQ ID NO: 229 - protein, 39 aa, Artificial sequence
  Synthetic sequence VPAC2 receptor peptide agonist
HSDAVFTDNYTXLRXQVAAXXYLQSIXXGGPSSGAPPPS

SEQ ID NO: 230 - protein, 39 aa, Artificial sequence
  Synthetic sequence VPAC2 receptor peptide agonist
HSDAVFTEQYTXLRXQVAAXXYLQSIXXGGPSSGAPPPC

SEQ ID NO: 231 - protein, 40 aa, Artificial sequence
  Synthetic sequence VPAC2 receptor peptide agonist
HSDAVFTDNYTRXRXQVAAXKYLQSIKXKGGPSSGAPPPS

SEQ ID NO: 232 - protein, 40 aa, Artificial sequence
  Synthetic sequence VPAC2 receptor peptide agonist
HSDAVFTDQYTRXRXQVAAXKYLQSIKXKGGPSSGAPPPS

SEQ ID NO: 233 - protein, 40 aa, Artificial sequence
  Synthetic sequence VPAC2 receptor peptide agonist
HSDAVFTDQYTRXRXQLAAXKYLQSIKXKGGPSSGAPPPS

SEQ ID NO: 234 - protein, 40 aa, Artificial sequence; identical to SEQ ID NO: 233
  Synthetic sequence VPAC2 receptor peptide agonist

SEQ ID NO: 235 - protein, 40 aa, Artificial sequence
  Synthetic sequence VPAC2 receptor peptide agonist
HSEAVFTEQYTRXRXQLAAXKYLQSIKXKGGPSSGAPPPS

SEQ ID NO: 236 - protein, 40 aa, Artificial sequence
  Synthetic sequence VPAC2 receptor peptide agonist
HSDAVFTDNYTXLRXQVAAXKYLQSIXNXGGPSSGAPPPS

SEQ ID NO: 237 - protein, 39 aa, Artificial sequence
  Synthetic sequence VPAC2 receptor peptide agonist
HSDAVFTEQYTXLRXQLAAXXYLQSIXXGGPSSGAPPPC

SEQ ID NO: 238 - protein, 39 aa, Artificial sequence; identical to SEQ ID NO: 55
  Synthetic sequence VPAC2 receptor peptide agonist

SEQ ID NO: 239 - protein, 39 aa, Artificial sequence
  Synthetic sequence VPAC2 receptor peptide agonist
HSDAVFTEQYTXLRXQVAAXXYLQSCXXGGPSSGAPPPS

SEQ ID NO: 240 - protein, 39 aa, Artificial sequence; identical to SEQ ID NO: 69
  Synthetic sequence VPAC2 receptor peptide agonist

SEQ ID NO: 241 - protein, 39 aa, Artificial sequence
  Synthetic sequence VPAC2 receptor peptide agonist
HSDAVFTEQYTXLRXQVAAXXYLQXIXXGGPSSGAPPPS

SEQ ID NO: 242 - protein, 39 aa, Artificial sequence
  Synthetic sequence VPAC2 receptor peptide agonist
HSDAVFTEQYTXLRXQVAAXXYLCSIXXGGPSSGAPPPS

SEQ ID NO: 243 - protein, 39 aa, Artificial sequence; identical to SEQ ID NO: 67
  Synthetic sequence VPAC2 receptor peptide agonist

SEQ ID NO: 244 - protein, 39 aa, Artificial sequence
  Synthetic sequence VPAC2 receptor peptide agonist
HSDAVFTEQYTXLRXQVACXXYLQSIXXGGPSSGAPPPS

SEQ ID NO: 245 - protein, 39 aa, Artificial sequence
  Synthetic sequence VPAC2 receptor peptide agonist
HSDAVFTEQYTXLRXQVAXXXYLQSIXXGGPSSGAPPPS

SEQ ID NO: 246 - protein, 39 aa, Artificial sequence
  Synthetic sequence VPAC2 receptor peptide agonist
HSDAVFTEQYTXLRXQCAAXXYLQSIXXGGPSSGAPPPS

SEQ ID NO: 247 - protein, 39 aa, Artificial sequence; identical to SEQ ID NO: 68
  Synthetic sequence VPAC2 receptor peptide agonist

SEQ ID NO: 248 - protein, 39 aa, Artificial sequence
  Synthetic sequence VPAC2 receptor peptide agonist
HSDAVFTEQYTXLRXCVAAXXYLQSIXXGGPSSGAPPPS

SEQ ID NO: 249 - protein, 39 aa, Artificial sequence
  Synthetic sequence VPAC2 receptor peptide agonist
HSDAVFTEQYTXLRXXVAAXXYLQSIXXGGPSSGAPPPS

SEQ ID NO: 250 - protein, 40 aa, Artificial sequence
  Synthetic sequence VPAC2 receptor peptide agonist
HSDAVFTEQYTXLRXQVAAXXYLQSIXCXGGPSSGAPPPS

SEQ ID NO: 251 - protein, 40 aa, Artificial sequence; identical to SEQ ID NO: 49
  Synthetic sequence VPAC2 receptor peptide agonist

SEQ ID NO: 252 - protein, 39 aa, Artificial sequence
  Synthetic sequence VPAC2 receptor peptide agonist
HSDAVFTEQYTXLRXQVAAXXYLQCIXXGGPSSGAPPPC

SEQ ID NO: 253 - protein, 39 aa, Artificial sequence
  Synthetic sequence VPAC2 receptor peptide agonist
HSDAVFTECYTXLRXQVAAXXYLQSIXXGGPSSGAPPPS

SEQ ID NO: 254 - protein, 39 aa, Artificial sequence
  Synthetic sequence VPAC2 receptor peptide agonist
HSDAVFTEQYTXCRXQVAAXXYLQSIXXGGPSSGAPPPS

SEQ ID NO: 255 - protein, 39 aa, Artificial sequence
  Synthetic sequence VPAC2 receptor peptide agonist
HSDAVFTEQYTXXRXQVAAXXYLQSIXXGGPSSGAPPPS

SEQ ID NO: 256 - protein, 39 aa, Artificial sequence
  Synthetic sequence VPAC2 receptor peptide agonist
HSDAVFTENYTKLRKQXAAKKYLNDLKKGGPSSGAPPPS